(19) United States
(12) Patent Application Publication
(10) Pub. No.: US 2023/0042547 A1
(43) Pub. Date: Feb. 9, 2023

(54) COMPOUND, ORGANIC ELECTROLUMINESCENCE DEVICE, AND ELECTRONIC APPARATUS

(71) Applicant: IDEMITSU KOSAN CO.,LTD., Tokyo (JP)

(72) Inventors: Kei YOSHIDA, Tokyo (JP); Masato MITANI, Tokyo (JP); Masatoshi SAITO, Tokyo (JP); Sayaka MIZUTANI, Tokyo (JP); Yoshinori AOYAMA, Tokyo (JP)

(73) Assignee: IDEMITSU KOSAN CO.,LTD., Tokyo (JP)

(21) Appl. No.: 17/843,290

(22) Filed: Jun. 17, 2022

(30) Foreign Application Priority Data
Jun. 30, 2021 (JP) 2021-109425

(51) Int. Cl. H01L 51/00; C07D 409/14

(52) U.S. Cl. CPC H01L 51/0067; C07D 409/14; H01L 51/0072; H01L 51/0074; H01L 51/508

(57) ABSTRACT

A compound represented by the following formula (1):

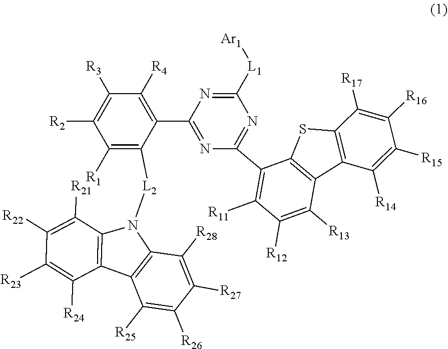

### TECHNICAL FIELD

Embodiments described herein generally relate to a novel compound, a material for an organic electroluminescence device, an organic electroluminescence device, and an electronic apparatus.

### BACKGROUND ART

When voltage is applied to an organic electroluminescence device (hereinafter, referred to as an organic EL device), holes and electrons are injected into an emitting layer from an anode and a cathode, respectively. Then, thus injected holes and electrons are recombined in the emitting layer, and excitons are formed therein.

The device performance of conventional organic EL devices is not sufficient yet. Although the improvement of the material used for the organic EL device is progressing gradually in order to raise the device performance, further performance enhancement is required.

Patent Document 1 discloses use of a compound having a specific structure in an organic layer of an organic EL device.

### RELATED ART DOCUMENTS

### Patent Documents

[Patent Document 1] WO 2019/098796

### SUMMARY OF THE INVENTION

It is an object of the invention to provide a compound capable of realizing an organic EL device having higher performance.

As a result of extensive studies to achieve the above object, the inventors have found that an organic EL device having higher performance can be obtained when a compound having a specific structure is used, and has completed the invention.

According to the invention, the following compound and so on are provided.

1. A compound represented by the following formula (1):

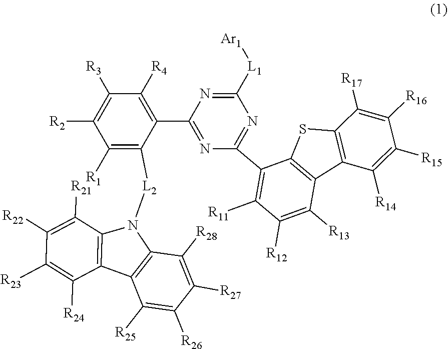

wherein in the formula (1),

Ar₁ is a substituted or unsubstituted aryl group including 6 to 50 ring carbon atoms;

L₁ and L₂ are independently a single bond or a substituted or unsubstituted arylene group including 6 to 50 ring carbon atoms;

R₁ to R₄ are a hydrogen atom, a substituted or unsubstituted alkyl group including 1 to 50 carbon atoms, or a substituted or unsubstituted aryl group including 6 to 50 ring carbon atoms; one or more sets of adjacent two or more of R₁ to R₄ are not bonded with each other;

one or more sets of adjacent two or more of R₁₁ to R₁₇ form a substituted or unsubstituted, saturated or unsaturated ring by bonding with each other, or do not form a substituted or unsubstituted, saturated or unsaturated ring;

R₁₁ to R₁₇ which do not form the substituted or unsubstituted, saturated or unsaturated ring are independently a hydrogen atom or a substituent R;

R₂₁ and R₂₂ form a substituted or unsubstituted, saturated or unsaturated ring by bonding with each other, or do not form a substituted or unsubstituted, saturated or unsaturated ring;

one or more sets of adjacent two or more of R₂₃ to R₂₆ form a substituted or unsubstituted, saturated or unsaturated ring by bonding with each other, or do not form a substituted or unsubstituted, saturated or unsaturated ring;

R₂₇ and R₂₈ form a substituted or unsubstituted, saturated or unsaturated ring by bonding with each other, or do not form a substituted or unsubstituted, saturated or unsaturated ring;

R₂₂ and R₂₃ are not bonded with each other;

R₂₆ and R₂₇ are not bonded with each other;

R₂₁ to R₂₈ which do not form the substituted or unsubstituted, saturated or unsaturated ring are independently a hydrogen atom or a substituent R;

the substituent R is selected from the group consisting of

a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),

### —O—(R₉₀₄),

### —S—(R₉₀₅),

—N(R₉₀₆)(R₉₀₇)
(where R₉₀₁ to R₉₀₇ are independently
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms; and when two or more
of each of R₉₀₁ to R₉₀₇ are present, the two or more of each of R₉₀₁ to R₉₀₇ may be the same as or different from each other),
a halogen atom, a cyano group, a nitro group,
a substituted or unsubstituted aryl group including 6 to 50 ring carbon atoms, and
a substituted or unsubstituted monovalent heterocyclic group including 5 to 50 ring atoms;

when two or more substituent R's are present, the two or more substituent R's may be the same as or different from each other.

According to the invention, the compound capable of realizing an organic EL device having higher performance can be provided.

### BRIEF DESCRIPTION OF THE DRAWINGS

The FIGURE is a diagram showing a schematic configuration of an aspect of an organic EL device according to an aspect of the invention.

### MODE FOR CARRYING OUT THE INVENTION

### Definition

In this specification, a hydrogen atom includes its isotopes different in the number of neutrons, namely, a protium, a deuterium and a tritium.

In this specification, at a bondable position in a chemical formula where a symbol such as “R”, or “D” representing a deuterium atom is not indicated, a hydrogen atom, that is, a protium atom, a deuterium atom or a tritium atom is bonded.

In this specification, the number of ring carbon atoms represents the number of carbon atoms forming a subject ring itself among the carbon atoms of a compound having a structure in which atoms are bonded in a ring form (for example, a monocyclic compound, a fused ring compound, a cross-linked compound, a carbocyclic compound, or a heterocyclic compound). When the subject ring is substituted by a substituent, the carbon contained in the substituent is not included in the number of ring carbon atoms. The same shall apply to “the number of ring carbon atoms” described below, unless otherwise specified. For example, a benzene ring has 6 ring carbon atoms, a naphthalene ring includes 10 ring carbon atoms, a pyridine ring includes 5 ring carbon atoms, and a furan ring includes 4 ring carbon atoms. Further, for example, a 9,9-diphenylfluorenyl group includes 13 ring carbon atoms, and a 9,9′-spirobifluorenyl group includes 25 ring carbon atoms.

When a benzene ring is substituted by, for example, an alkyl group as a substituent, the number of carbon atoms of the alkyl group is not included in the number of ring carbon atoms of the benzene ring. Therefore, the number of ring carbon atoms of the benzene ring substituted by the alkyl group is 6. When a naphthalene ring is substituted by, for example, an alkyl group as a substituent, the number of carbon atoms of the alkyl group is not included in the number of ring carbon atoms of the naphthalene ring. Therefore, the number of ring carbon atoms of the naphthalene ring substituted by the alkyl group is 10.

In this specification, the number of ring atoms represents the number of atoms forming a subject ring itself among the atoms of a compound having a structure in which atoms are bonded in a ring form (for example, the structure includes a monocyclic ring, a fused ring and a ring assembly) (for example, a monocyclic compound, a fused ring compound, a cross-linked compound, a carbocyclic compound and a heterocyclic compound). The number of ring atoms does not include atoms which do not form the ring (for example, a hydrogen atom which terminates a bond of the atoms forming the ring), or atoms contained in a substituent when the ring is substituted by the substituent. The same shall apply to “the number of ring atoms” described below, unless otherwise specified. For example, the number of atoms of a pyridine ring is 6, the number of atoms of a quinazoline ring is 10, and the number of a furan ring is 5. For example, hydrogen atoms bonded to a pyridine ring and atoms constituting a substituent substituted on the pyridine ring are not included in the number of ring atoms of the pyridine ring. Therefore, the number of ring atoms of a pyridine ring with which a hydrogen atom or a substituent is bonded is 6. For example, hydrogen atoms and atoms constituting a substituent which are bonded with a quinazoline ring is not included in the number of ring atoms of the quinazoline ring. Therefore, the number of ring atoms of a quinazoline ring with which a hydrogen atom or a substituent is bonded is 10.

In this specification, “XX to YY carbon atoms” in the expression “a substituted or unsubstituted ZZ group including XX to YY carbon atoms” represents the number of carbon atoms in the case where the ZZ group is unsubstituted by a substituent, and does not include the number of carbon atoms of a substituent in the case where the ZZ group is substituted by the substituent. Here, “YY” is larger than “XX”, and “XX” means an integer of 1 or more and “YY” means an integer of 2 or more.

In this specification, “XX to YY atoms” in the expression “a substituted or unsubstituted ZZ group including XX to YY atoms” represents the number of atoms in the case where the ZZ group is unsubstituted by a substituent, and does not include the number of atoms of a substituent in the case where the ZZ group is substituted by the substituent. Here, “YY” is larger than “XX”, and “XX” means an integer of 1 or more and “YY” means an integer of 2 or more.

In this specification, the unsubstituted ZZ group represents the case where the “substituted or unsubstituted ZZ group” is a “ZZ group unsubstituted by a substituent”, and the substituted ZZ group represents the case where the “substituted or unsubstituted ZZ group” is a “ZZ group substituted by a substituent”.

In this specification, a term “unsubstituted” in the case of “a substituted or unsubstituted ZZ group” means that hydrogen atoms in the ZZ group are not substituted by a substituent. Hydrogen atoms in a term “unsubstituted ZZ group” are a protium atom, a deuterium atom, or a tritium atom.

In this specification, a term “substituted” in the case of “a substituted or unsubstituted ZZ group” means that one or more hydrogen atoms in the ZZ group are substituted by a substituent. Similarly, a term “substituted” in the case of “a BB group substituted by an AA group” means that one or more hydrogen atoms in the BB group are substituted by the AA group.

“Substituent as Described in this Specification”

Hereinafter, the substituent described in this specification will be explained.

The number of ring carbon atoms of the “unsubstituted aryl group” described in this specification is 6 to 50, preferably 6 to 30, and more preferably 6 to 18, unless otherwise specified.

The number of ring atoms of the “unsubstituted heterocyclic group” described in this specification is 5 to 50, preferably 5 to 30, and more preferably 5 to 18, unless otherwise specified.

The number of carbon atoms of the “unsubstituted alkyl group” described in this specification is 1 to 50, preferably 1 to 20, and more preferably 1 to 6, unless otherwise specified.

The number of carbon atoms of the “unsubstituted alkenyl group” described in this specification is 2 to 50, preferably 2 to 20, and more preferably 2 to 6, unless otherwise specified.

The number of carbon atoms of the “unsubstituted alkynyl group” described in this specification is 2 to 50, preferably 2 to 20, and more preferably 2 to 6, unless otherwise specified.

The number of ring carbon atoms of the “unsubstituted cycloalkyl group” described in this specification is 3 to 50, preferably 3 to 20, and more preferably 3 to 6, unless otherwise specified.

The number of ring carbon atoms of the “unsubstituted arylene group” described in this specification is 6 to 50, preferably 6 to 30, and more preferably 6 to 18, unless otherwise specified.

The number of ring atoms of the “unsubstituted divalent heterocyclic group” described in this specification is 5 to 50, preferably 5 to 30, and more preferably 5 to 18, unless otherwise specified.

The number of carbon atoms of the “unsubstituted alkylene group” described in this specification is 1 to 50, preferably 1 to 20, and more preferably 1 to 6, unless otherwise specified.

“Substituted or Unsubstituted Aryl Group”

Specific examples of the “substituted or unsubstituted aryl group” described in this specification (specific example group G1) include the following unsubstituted aryl groups (specific example group G1A), substituted aryl groups (specific example group G1B), and the like. (Here, the unsubstituted aryl group refers to the case where the “substituted or unsubstituted aryl group” is an “aryl group unsubstituted by a substituent”, and the substituted aryl group refers to the case where the “substituted or unsubstituted aryl group” is an “aryl group substituted by a substituent”). In this specification, in the case where simply referred as an “aryl group”, it includes both a “unsubstituted aryl group” and a “substituted aryl group.”

The “substituted aryl group” means a group in which one or more hydrogen atoms of the “unsubstituted aryl group” are substituted by a substituent. Specific examples of the “substituted aryl group” include, for example, groups in which one or more hydrogen atoms of the “unsubstituted aryl group” of the following specific example group G1A are substituted by a substituent, the substituted aryl groups of the following specific example group G1B, and the like. It should be noted that the examples of the “unsubstituted aryl group” and the examples of the “substituted aryl group” enumerated in this specification are mere examples, and the “substituted aryl group” described in this specification also includes a group in which a hydrogen atom bonded with a carbon atom of the aryl group itself in the “substituted aryl group” of the following specific group G1B is further substituted by a substituent, and a group in which a hydrogen atom of a substituent in the “substituted aryl group” of the following specific group G1B is further substituted by a substituent.

Unsubstituted Aryl Group (Specific Example Group G1A):

a phenyl group,

a p-biphenyl group,

a m-biphenyl group,

an o-biphenyl group,

a p-terphenyl-4-yl group,

a p-terphenyl-3-yl group,

a p-terphenyl-2-yl group,

a m-terphenyl-4-yl group,

a m-terphenyl-3-yl group,

a m-terphenyl-2-yl group,

an o-terphenyl-4-yl group,

an o-terphenyl-3-yl group,

an o-terphenyl-2-yl group,

a 1-naphthyl group,

a 2-naphthyl group,

an anthryl group,

a benzanthryl group,

a phenanthryl group,

a benzophenanthryl group,

a phenalenyl group,

a pyrenyl group,

a chrysenyl group,

a benzochrysenyl group,

a triphenylenyl group,

a benzotriphenylenyl group,

a tetracenyl group,

a pentacenyl group,

a fluorenyl group,

a 9,9′-spirobifluorenyl group,

a benzofluorenyl group,

a dibenzofluorenyl group,

a fluoranthenyl group,

a benzofluoranthenyl group,

a perylenyl group, and

a monovalent aryl group derived by removing one hydrogen atom from the ring structures represented by each of the following general formulas (TEMP-1) to (TEMP-15).

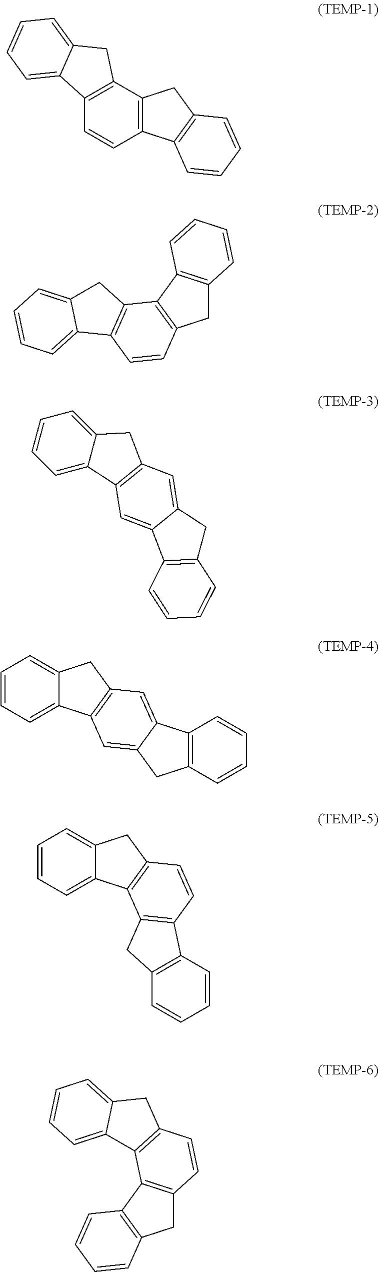

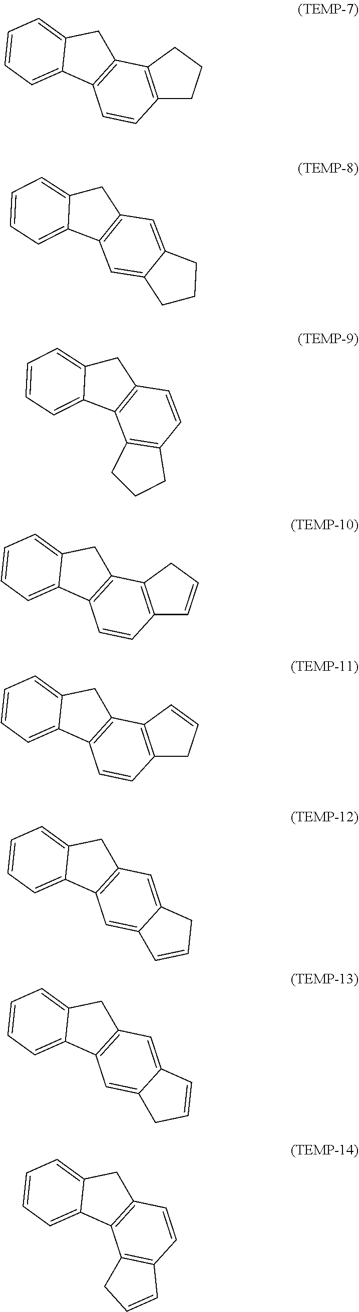

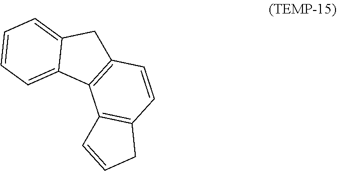

Substituted Aryl Group (Specific Example Group G1B):

an o-tolyl group,

a m-tolyl group,

a p-tolyl group,

a p-xylyl group,

a m-xylyl group,

an o-xylyl group,

a p-isopropylphenyl group,

a m-isopropylphenyl group,

an o-isopropylphenyl group,

a p-t-butylphenyl group,

a m-t-butylphenyl group,

an o-t-butylphenyl group,

a 3,4,5-trimethylphenyl group,

a 9,9-dimethylfluorenyl group,

a 9,9-diphenylfluorenyl group,

a 9,9-bis(4-methylphenyl)fluorenyl group,

a 9,9-bis(4-isopropylphenyl)fluorenyl group,

a 9,9-bis(4-t-butylphenyl)fluorenyl group,

a cyanophenyl group,

a triphenylsilylphenyl group,

a trimethylsilylphenyl group,

a phenylnaphthyl group,

a naphthylphenyl group, and

a group in which one or more hydrogen atoms of a monovalent group derived from the ring structures represented by each of the general formulas (TEMP-1) to (TEMP-15) are substituted by a substituent.

“Substituted or Unsubstituted Heterocyclic Group”

The “heterocyclic group” described in this specification is a ring group having at least one hetero atom in the ring atom. Specific examples of the hetero atom include a nitrogen atom, an oxygen atom, a sulfur atom, a silicon atom, a phosphorus atom, and a boron atom.

The “heterocyclic group” in this specification is a monocyclic group or a fused ring group.

The “heterocyclic group” in this specification is an aromatic heterocyclic group or a non-aromatic heterocyclic group.

Specific examples of the “substituted or unsubstituted heterocyclic group” (specific example group G2) described in this specification include the following unsubstituted heterocyclic group (specific example group G2A), the following substituted heterocyclic group (specific example group G2B), and the like. (Here, the unsubstituted heterocyclic group refers to the case where the “substituted or unsubstituted heterocyclic group” is a “heterocyclic group unsubstituted by a substituent”, and the substituted heterocyclic group refers to the case where the “substituted or unsubstituted heterocyclic group” is a “heterocyclic group substituted by a substituent”). In this specification, in the case where simply referred as a “heterocyclic group”, it includes both the “unsubstituted heterocyclic group” and the “substituted heterocyclic group.”

The “substituted heterocyclic group” means a group in which one or more hydrogen atom of the “unsubstituted heterocyclic group” are substituted by a substituent. Specific examples of the “substituted heterocyclic group” include a group in which a hydrogen atom of “unsubstituted heterocyclic group” of the following specific example group G2A is substituted by a substituent, the substituted heterocyclic groups of the following specific example group G2B, and the like. It should be noted that the examples of the “unsubstituted heterocyclic group” and the examples of the “substituted heterocyclic group” enumerated in this specification are mere examples, and the “substituted heterocyclic group” described in this specification includes groups in which hydrogen atom bonded with a ring atom of the heterocyclic group itself in the “substituted heterocyclic group” of the specific example group G2B is further substituted by a substituent, and a group in which hydrogen atom of a substituent in the “substituted heterocyclic group” of the specific example group G2B is further substituted by a substituent.

Specific example group G2A includes, for example, the following unsubstituted heterocyclic group containing a nitrogen atom (specific example group G2A1), the following unsubstituted heterocyclic group containing an oxygen atom (specific example group G2A2), the following unsubstituted heterocyclic group containing a sulfur atom (specific example group G2A3), and the monovalent heterocyclic group derived by removing one hydrogen atom from the ring structures represented by each of the following general formulas (TEMP-16) to (TEMP-33) (specific example group G2A4).

Specific example group G2B includes, for example, the following substituted heterocyclic group containing a nitrogen atom (specific example group G2B1), the following substituted heterocyclic group containing an oxygen atom (specific example group G2B2), the following substituted heterocyclic group containing a sulfur atom (specific example group G2B3), and the following group in which one or more hydrogen atoms of the monovalent heterocyclic group derived from the ring structures represented by each of the following general formulas (TEMP-16) to (TEMP-33) are substituted by a substituent (specific example group G2B4).

Unsubstituted Heterocyclic Group Containing a Nitrogen Atom (Specific Example Group G2A1):

a pyrrolyl group,

an imidazolyl group,

a pyrazolyl group,

a triazolyl group,

a tetrazolyl group,

an oxazolyl group,

an isoxazolyl group,

an oxadiazolyl group,

a thiazolyl group,

an isothiazolyl group,

a thiadiazolyl group,

a pyridyl group,

a pyridazinyl group,

a pyrimidinyl group,

a pyrazinyl group,

a triazinyl group,

an indolyl group,

an isoindolyl group,

an indolizinyl group,

a quinolizinyl group,

a quinolyl group,

an isoquinolyl group,

a cinnolyl group,

a phthalazinyl group,

a quinazolinyl group,

a quinoxalinyl group,

a benzimidazolyl group,

an indazolyl group,

a phenanthrolinyl group,

a phenanthridinyl group,

an acridinyl group,

a phenazinyl group,

a carbazolyl group,

a benzocarbazolyl group,

a morpholino group,

a phenoxazinyl group,

a phenothiazinyl group,

an azacarbazolyl group, and

a diazacarbazolyl group.

Unsubstituted Heterocyclic Group Containing an Oxygen Atom (Specific Example Group G2A2):

a furyl group,

an oxazolyl group,

an isoxazolyl group,

an oxadiazolyl group,

a xanthenyl group,

a benzofuranyl group,

an isobenzofuranyl group,

a dibenzofuranyl group,

a naphthobenzofuranyl group,

a benzoxazolyl group,

a benzisoxazolyl group,

a phenoxazinyl group,

a morpholino group,

a dinaphthofuranyl group,

an azadibenzofuranyl group,

a diazadibenzofuranyl group,

an azanaphthobenzofuranyl group, and

a diazanaphthobenzofuranyl group.

Unsubstituted Heterocyclic Group Containing a Sulfur Atom (Specific Example Group G2A3):

a thienyl group,

a thiazolyl group,

an isothiazolyl group,

a thiadiazolyl group,

a benzothiophenyl group (benzothienyl group),

an isobenzothiophenyl group (isobenzothienyl group),

a dibenzothiophenyl group (dibenzothienyl group),

a naphthobenzothiophenyl group (naphthobenzothienyl group),

a benzothiazolyl group,

a benzisothiazolyl group,

a phenothiazinyl group,

a dinaphthothiophenyl group (dinaphthothienyl group),

an azadibenzothiophenyl group (azadibenzothienyl group),

a diazadibenzothiophenyl group (diazadibenzothienyl group),

an azanaphthobenzothiophenyl group (azanaphthobenzothienyl group), and

a diazanaphthobenzothiophenyl group (diazanaphthobenzothienyl group).

Monovalent Heterocyclic Group Derived by Removing One Hydrogen Atom from the Ring Structures Represented by Each of the Following General Formulas (TEMP-16) to (TEMP-33) (Specific Example Group G2A4):

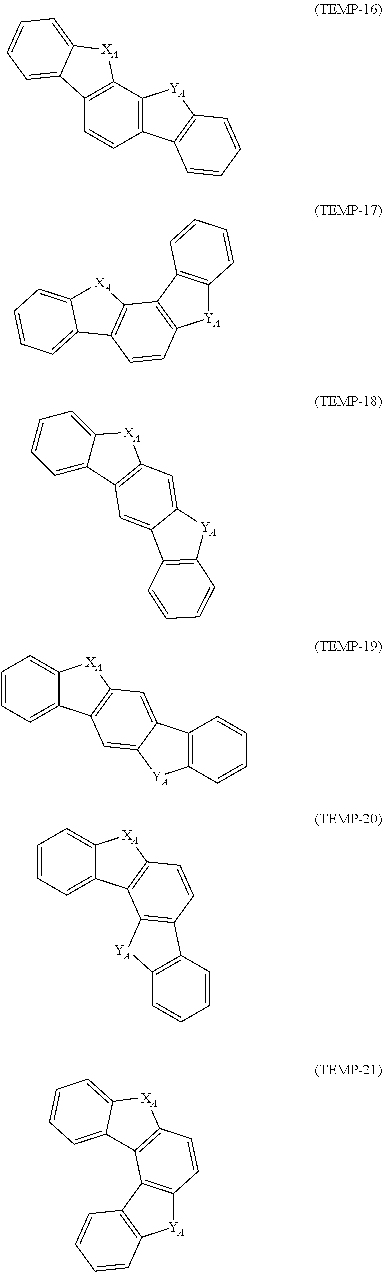

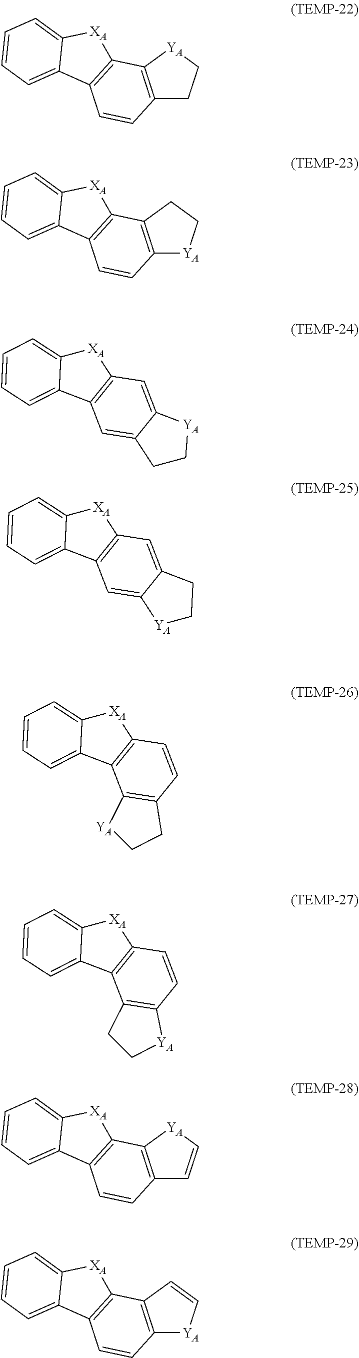

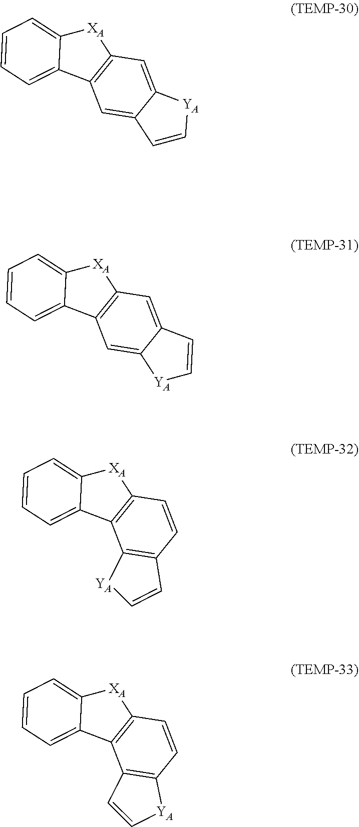

In the general formulas (TEMP-16) to (TEMP-33), X_{A} and Y_{A} are independently an oxygen atom, a sulfur atom, NH, or CH₂. Provided that at least one of X_{A} and Y_{A} is an oxygen atom, a sulfur atom, or NH.

In the general formulas (TEMP-16) to (TEMP-33), when at least one of X_{A} and Y_{A} is NH or CH₂, the monovalent heterocyclic group derived from the ring structures represented by each of the general formulas (TEMP-16) to (TEMP-33) includes a monovalent group derived by removing one hydrogen atom from these NH or CH₂.

Substituted Heterocyclic Group Containing a Nitrogen Atom (Specific Example Group G2B1):

a (9-phenyl)carbazolyl group,

a (9-biphenylyl)carbazolyl group,

a (9-phenyl)phenylcarbazolyl group,

a (9-naphthyl)carbazolyl group,

a diphenylcarbazol-9-yl group,

a phenylcarbazol-9-yl group,

a methylbenzimidazolyl group,

an ethylbenzimidazolyl group,

a phenyltriazinyl group,

a biphenylyltriazinyl group,

a diphenyltriazinyl group,

a phenylquinazolinyl group, and

a biphenylylquinazolinyl group.

Substituted Heterocyclic Group Containing an Oxygen Atom (Specific Example Group G2B2):

a phenyldibenzofuranyl group,

a methyldibenzofuranyl group,

a t-butyldibenzofuranyl group, and

a monovalent residue of spiro[9H-xanthene-9,9′-[9H]fluorene].

Substituted Heterocyclic Group Containing a Sulfur Atom (Specific Example Group G2B3):

a phenyldibenzothiophenyl group,

a methyldibenzothiophenyl group,

a t-butyldibenzothiophenyl group, and

a monovalent residue of spiro[9H-thioxanthene-9,9′-[9H]fluorene].

Group in which One or More Hydrogen Atoms of the Monovalent Heterocyclic Group Derived from the Ring Structures Represented by Each of the Following General Formulas (TEMP-16) to (TEMP-33) are Substituted by a Substituent (Specific Example Group G2B4):

The “one or more hydrogen atoms of the monovalent heterocyclic group” means one or more hydrogen atoms selected from hydrogen atoms bonded with ring carbon atoms of the monovalent heterocyclic group, a hydrogen atom bonded with a nitrogen atom when at least one of X_{A} and Y_{A} is NH, and hydrogen atoms of a methylene group when one of X_{A} and Y_{A} is CH₂.

“Substituted or Unsubstituted Alkyl Group”

Specific examples of the “substituted or unsubstituted alkyl group” (specific example group G3) described in this specification include the following unsubstituted alkyl groups (specific example group G3A) and the following substituted alkyl groups (specific example group G3B). (Here, the unsubstituted alkyl group refers to the case where the “substituted or unsubstituted alkyl group” is an “alkyl group unsubstituted by a substituent”, and the substituted alkyl group refers to the case where the “substituted or unsubstituted alkyl group” is an “alkyl group substituted by a substituent”). In this specification, in the case where simply referred as an “alkyl group” includes both the “unsubstituted alkyl group” and the “substituted alkyl group.”

The “substituted alkyl group” means a group in which one or more hydrogen atoms in the “unsubstituted alkyl group” are substituted by a substituent. Specific examples of the “substituted alkyl group” include groups in which one or more hydrogen atoms in the following “unsubstituted alkyl group” (specific example group G3A) are substituted by a substituent, the following substituted alkyl group (specific example group G3B), and the like. In this specification, the alkyl group in the “unsubstituted alkyl group” means a linear alkyl group. Thus, the “unsubstituted alkyl group” includes a straight-chain “unsubstituted alkyl group” and a branched-chain “unsubstituted alkyl group”. It should be noted that the examples of the “unsubstituted alkyl group” and the examples of the “substituted alkyl group” enumerated in this specification are mere examples, and the “substituted alkyl group” described in this specification includes a group in which hydrogen atom of the alkyl group itself in the “substituted alkyl group” of the specific example group G3B is further substituted by a substituent, and a group in which hydrogen atom of a substituent in the “substituted alkyl group” of the specific example group G3B is further substituted by a substituent.

Unsubstituted Alkyl Group (Specific Example Group G3A):

a methyl group,

an ethyl group,

a n-propyl group,

an isopropyl group,

a n-butyl group,

an isobutyl group,

a s-butyl group, and

a t-butyl group.

Substituted Alkyl Group (Specific Example Group G3B):

a heptafluoropropyl group (including isomers),

a pentafluoroethyl group,

a 2,2,2-trifluoroethyl group, and

a trifluoromethyl group.

“Substituted or Unsubstituted Alkenyl Group”

Specific examples of the “substituted or unsubstituted alkenyl group” described in this specification (specific example group G4) include the following unsubstituted alkenyl group (specific example group G4A), the following substituted alkenyl group (specific example group G4B), and the like. (Here, the unsubstituted alkenyl group refers to the case where the “substituted or unsubstituted alkenyl group” is a “alkenyl group unsubstituted by a substituent”, and the “substituted alkenyl group” refers to the case where the “substituted or unsubstituted alkenyl group” is a “alkenyl group substituted by a substituent.”). In this specification, in the case where simply referred as an “alkenyl group” includes both the “unsubstituted alkenyl group” and the “substituted alkenyl group.”

The “substituted alkenyl group” means a group in which one or more hydrogen atoms in the “unsubstituted alkenyl group” are substituted by a substituent. Specific examples of the “substituted alkenyl group” include a group in which the following “unsubstituted alkenyl group” (specific example group G4A) has a substituent, the following substituted alkenyl group (specific example group G4B), and the like. It should be noted that the examples of the “unsubstituted alkenyl group” and the examples of the “substituted alkenyl group” enumerated in this specification are mere examples, and the “substituted alkenyl group” described in this specification includes a group in which a hydrogen atom of the alkenyl group itself in the “substituted alkenyl group” of the specific example group G4B is further substituted by a substituent, and a group in which a hydrogen atom of a substituent in the “substituted alkenyl group” of the specific example group G4B is further substituted by a substituent.

Unsubstituted Alkenyl Group (Specific Example Group G4A):

a vinyl group,

an allyl group,

a 1-butenyl group,

a 2-butenyl group, and

a 3-butenyl group.

Substituted Alkenyl Group (Specific Example Group G4B):

a 1,3-butanedienyl group,

a 1-methylvinyl group,

a 1-methylallyl group,

a 1,1-dimethylallyl group,

a 2-methylally group, and

a 1,2-dimethylallyl group.

“Substituted or Unsubstituted Alkynyl Group”

Specific examples of the “substituted or unsubstituted alkynyl group” described in this specification (specific example group G5) include the following unsubstituted alkynyl group (specific example group G5A) and the like. (Here, the unsubstituted alkynyl group refers to the case where the “substituted or unsubstituted alkynyl group” is an “alkynyl group unsubstituted by a substituent”). In this specification, in the case where simply referred as an “alkynyl group” includes both the “unsubstituted alkynyl group” and the “substituted alkynyl group.”

The “substituted alkynyl group” means a group in which one or more hydrogen atoms in the “unsubstituted alkynyl group” are substituted by a substituent. Specific examples of the “substituted alkynyl group” include a group in which one or more hydrogen atoms in the following “unsubstituted alkynyl group” (specific example group G5A) are substituted by a substituent, and the like.

Unsubstituted Alkynyl Group (Specific Example Group G5A):

an ethynyl group.

“Substituted or Unsubstituted Cycloalkyl Group”

Specific examples of the “substituted or unsubstituted cycloalkyl group” described in this specification (specific example group G6) include the following unsubstituted cycloalkyl group (specific example group G6A), the following substituted cycloalkyl group (specific example group G6B), and the like. (Here, the unsubstituted cycloalkyl group refers to the case where the “substituted or unsubstituted cycloalkyl group” is a “cycloalkyl group unsubstituted by a substituent”, and the substituted cycloalkyl group refers to the case where the “substituted or unsubstituted cycloalkyl group” is a “cycloalkyl group substituted by a substituent”). In this specification, in the case where simply referred as a “cycloalkyl group” includes both the “unsubstituted cycloalkyl group” and the “substituted cycloalkyl group.”

The “substituted cycloalkyl group” means a group in which one or more hydrogen atoms in the “unsubstituted cycloalkyl group” are substituted by a substituent. Specific examples of the “substituted cycloalkyl group” include a group in which one or more hydrogen atoms in the following “unsubstituted cycloalkyl group” (specific example group G6A) are substituted by a substituent, and examples of the following substituted cycloalkyl group (specific example group G6B), and the like. It should be noted that the examples of the “unsubstituted cycloalkyl group” and the examples of the “substituted cycloalkyl group” enumerated in this specification are mere examples, and the “substituted cycloalkyl group” in this specification includes a group in which one or more hydrogen atoms bonded with the carbon atom of the cycloalkyl group itself in the “substituted cycloalkyl group” of the specific example group G6B are substituted by a substituent, and a group in which a hydrogen atom of a substituent in the “substituted cycloalkyl group” of specific example group G6B is further substituted by a substituent.

Unsubstituted Cycloalkyl Group (Specific Example Group G6A):

a cyclopropyl group,

a cyclobutyl group,

a cyclopentyl group,

a cyclohexyl group,

a 1-adamantyl group,

a 2-adamantyl group,

a 1-norbornyl group, and

a 2-norbornyl group.

Substituted Cycloalkyl Group (Specific Example Group G6B):

a 4-methylcyclohexyl group.

“Group represented by —Si(R₉₀₁)(R₉₀₂)(R₉₀₃)”

Specific examples of the group represented by —Si(R₉₀₁)(R₉₀₂)(R₉₀₃) described in this specification (specific example group G7) include:

—Si(G1)(G1)(G1),

—Si(G1)(G2)(G2),

—Si(G1)(G1)(G2),

—Si(G2)(G2)(G2),

—Si(G3)(G3)(G3), and

—Si(G6)(G6)(G6).

G1 is the “substituted or unsubstituted aryl group” described in the specific example group G1.

G2 is the “substituted or unsubstituted heterocyclic group” described in the specific example group G2.

G3 is the “substituted or unsubstituted alkyl group” described in the specific example group G3.

G6 is the “substituted or unsubstituted cycloalkyl group” described in the specific example group G6.

Plural G1's in —Si(G1)(G1)(G1) are the same or different.

Plural G2's in —Si(G1)(G2)(G2) are the same or different.

Plural G1's in —Si(G1)(G1)(G2) are the same or different.

Plural G2's in —Si(G2)(G2)(G2) are be the same or different.

Plural G3's in —Si(G3)(G3)(G3) are the same or different.

Plural G6's in —Si(G6)(G6)(G6) are be the same or different.

“Group Represented by —O—(R₉₀₄)”

Specific examples of the group represented by —O—(R₉₀₄) in this specification (specific example group G8) include:

—O(G1),

—O(G2),

—O(G3), and

—O(G6).

G1 is the “substituted or unsubstituted aryl group” described in the specific example group G1.

G2 is the “substituted or unsubstituted heterocyclic group” described in the specific example group G2.

G3 is the “substituted or unsubstituted alkyl group” described in the specific example group G3.

G6 is the “substituted or unsubstituted cycloalkyl group” described in the specific example group G6.

“Group represented by —S—(R₉₀₅)”

Specific examples of the group represented by —S—(R₉₀₅) in this specification (specific example group G9) include:

—S(G1),

—S(G2),

—S(G3), and

—S(G6).

G1 is the “substituted or unsubstituted aryl group” described in the specific example group G1.

G2 is the “substituted or unsubstituted heterocyclic group” described in the specific example group G2.

G3 is the “substituted or unsubstituted alkyl group” described in the specific example group G3.

G6 is the “substituted or unsubstituted cycloalkyl group” described in the specific example group G6.

“Group represented by —N(R₉₀₆)(R₉₀₇)”

Specific examples of the group represented by —N(R₉₀₆)(R₉₀₇) in this specification (specific example group G10) include:

—N(G1)(G1),

—N(G2)(G2),

—N(G1)(G2),

—N(G3)(G3), and

—N(G6)(G6).

G1 is the “substituted or unsubstituted aryl group” described in the specific example group G1.

G2 is the “substituted or unsubstituted heterocyclic group” described in the specific example group G2.

G3 is the “substituted or unsubstituted alkyl group” described in the specific example group G3.

G6 is the “substituted or unsubstituted cycloalkyl group” described in the specific example group G6.

Plural G1's in —N(G1)(G1) are the same or different.

Plural G2's in —N(G2)(G2) are the same or different.

Plural G3's in —N(G3)(G3) are the same or different.

Plural G6's in —N(G6)(G6) are the same or different.

“Halogen Atom”

Specific examples of the “halogen atom” described in this specification (specific example group G11) include a fluorine atom, a chlorine atom, a bromine atom, an iodine atom, and the like.

“Substituted or Unsubstituted Fluoroalkyl Group”

The “substituted or unsubstituted fluoroalkyl group” described in this specification is a group in which at least one hydrogen atom bonded with a carbon atom constituting the alkyl group in the “substituted or unsubstituted alkyl group” is substituted by a fluorine atom, and includes a group in which all hydrogen atoms bonded with a carbon atom constituting the alkyl group in the “substituted or unsubstituted alkyl group” are substituted by a fluorine atom (a perfluoro group). The number of carbon atoms of the “unsubstituted fluoroalkyl group” is 1 to 50, preferably 1 to 30, more preferably 1 to 18, unless otherwise specified in this specification. The “substituted fluoroalkyl group” means a group in which one or more hydrogen atoms of the “fluoroalkyl group” are substituted by a substituent. The “substituted fluoroalkyl group” described in this specification also includes a group in which one or more hydrogen atoms bonded with a carbon atom of the alkyl chains in the “substituted fluoroalkyl group” are further substituted by a substituent, and a group in which one or more hydrogen atom of a substituent in the “substituted fluoroalkyl group” are further substituted by a substituent. Specific examples of the “unsubstituted fluoroalkyl group” include a group in which one or more hydrogen atoms in the “alkyl group” (specific group G3) are substituted by a fluorine atom, and the like.

“Substituted or Unsubstituted Haloalkyl Group”

The “substituted or unsubstituted haloalkyl group” described in this specification is a group in which at least one hydrogen atom bonded with a carbon atom constituting the alkyl group in the “substituted or unsubstituted alkyl group” is substituted by a halogen atom, and also includes a group in which all hydrogen atoms bonded with a carbon atom constituting the alkyl group in the “substituted or unsubstituted alkyl group” are substituted by a halogen atom. The number of carbon atoms of the “unsubstituted haloalkyl group” is 1 to 50, preferably 1 to 30, more preferably 1 to 18, unless otherwise specified in this specification. The “substituted haloalkyl group” means a group in which one or more hydrogen atoms of the “haloalkyl group” are substituted by a substituent. The “substituted haloalkyl group” described in this specification also includes a group in which one or more hydrogen atoms bonded with a carbon atom of the alkyl chain in the “substituted haloalkyl group” are further substituted by a substituent, and a group in which one or more hydrogen atoms of a substituent in the “substituted haloalkyl group” are further substituted by a substituent. Specific examples of the “unsubstituted haloalkyl group” include a group in which one or more hydrogen atoms in the “alkyl group” (specific example group G3) are substituted by a halogen atom, and the like. A haloalkyl group is sometimes referred to as an alkyl halide group.

“Substituted or Unsubstituted Alkoxy Group”

Specific examples of the “substituted or unsubstituted alkoxy group” described in this specification include a group represented by —O(G3), wherein G3 is the “substituted or unsubstituted alkyl group” described in the specific example group G3. The number of carbon atoms of the “unsubstituted alkoxy group” is 1 to 50, preferably 1 to 30, more preferably 1 to 18, unless otherwise specified in this specification.

“Substituted or Unsubstituted Alkylthio Group”

Specific examples of the “substituted or unsubstituted alkylthio group” described in this specification include a group represented by —S(G3), wherein G3 is the “substituted or unsubstituted alkyl group” described in the specific example group G3. The number of carbon atoms of the “unsubstituted alkylthio group” is 1 to 50, preferably 1 to 30, more preferably 1 to 18, unless otherwise specified in this specification.

“Substituted or Unsubstituted Aryloxy Group”

Specific examples of the “substituted or unsubstituted aryloxy group” described in this specification include a group represented by —O(G1), wherein G1 is the “substituted or unsubstituted aryl group” described in the specific example group G1. The number of ring carbon atoms of the “unsubstituted aryloxy group” is 6 to 50, preferably 6 to 30, more preferably 6 to 18, unless otherwise specified in this specification.

“Substituted or Unsubstituted Arylthio Group”

Specific examples of the “substituted or unsubstituted arylthio group” described in this specification include a group represented by —S(G1), wherein G1 is a “substituted or unsubstituted aryl group” described in the specific example group G1. The number of ring carbon atoms of the “unsubstituted arylthio group” is 6 to 50, preferably 6 to 30, more preferably 6 to 18, unless otherwise specified in this specification.

“Substituted or Unsubstituted Trialkylsilyl Group”

Specific examples of the “trialkylsilyl group” described in this specification include a group represented by —Si(G3)(G3)(G3), where G3 is the “substituted or unsubstituted alkyl group” described in the specific example group G3. Plural G3's in —Si(G3)(G3)(G3) are the same or different. The number of carbon atoms in each alkyl group of the “trialkylsilyl group” is 1 to 50, preferably 1 to 20, more preferably 1 to 6, unless otherwise specified in this specification.

“Substituted or Unsubstituted Aralkyl Group”

Specific examples of the “substituted or unsubstituted aralkyl group” described in this specification is a group represented by -(G3)-(G1), wherein G3 is the “substituted or unsubstituted alkyl group” described in the specific example group G3, and G1 is the “substituted or unsubstituted aryl group” described in the specific example group G1. Therefore, the “aralkyl group” is a group in which a hydrogen atom of the “alkyl group” is substituted by an “aryl group” as a substituent, and is one form of the “substituted alkyl group.” The “unsubstituted aralkyl group” is the “unsubstituted alkyl group” substituted by the “unsubstituted aryl group”, and the number of carbon atoms of the “unsubstituted aralkyl group” is 7 to 50, preferably 7 to 30, more preferably 7 to 18, unless otherwise specified in this specification.

Specific examples of the “substituted or unsubstituted aralkyl group” include a benzyl group, a 1-phenylethyl group, a 2-phenylethyl group, a 1-phenylisopropyl group, a 2-phenylisopropyl group, a phenyl-t-butyl group, an α-naphthylmethyl group, a 1-α-naphthylethyl group, a 2-α-naphthylethyl group, a 1-α-naphthylisopropyl group, a 2-α-naphthylisopropyl group, a β-naphthylmethyl group, a 1-β-naphthylethyl group, a 2-β-naphthylethyl group, a 1-β-naphthylisopropyl group, a 2-β-naphthylisopropyl group, and the like.

Unless otherwise specified in this specification, examples of the substituted or unsubstituted aryl group described in this specification preferably include a phenyl group, a p-biphenyl group, a m-biphenyl group, an o-biphenyl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, an o-terphenyl-4-yl group, an o-terphenyl-3-yl group, an o-terphenyl-2-yl group, a 1-naphthyl group, a 2-naphthyl group, an anthryl group, a phenanthryl group, a pyrenyl group, a chrysenyl group, a triphenylenyl group, a fluorenyl group, a 9,9′-spirobifluorenyl group, 9,9-dimethylfluorenyl group, 9,9-diphenylfluorenyl group, and the like.

Unless otherwise specified in this specification, examples of the substituted or unsubstituted heterocyclic groups described in this specification preferably include a pyridyl group, a pyrimidinyl group, a tiazinyl group, a quinolyl group, an isoquinolyl group, a quinazolinyl group, a benzimidazolyl group, a phenanthrolinyl group, a carbazolyl group (a 1-carbazolyl group, a 2-carbazolyl group, a 3-carbazolyl group, a 4-carbazolyl group, or a 9-carbazolyl group), a benzocarbazolyl group, an azacarbazolyl group, a diazacarbazolyl group, a dibenzofuranyl group, a naphthobenzofuranyl group, an azadibenzofuranyl group, a diazadibenzofuranyl group, a dibenzothiophenyl group, a naphthobenzothiophenyl group, an azadibenzothiophenyl group, a diazadibenzothiophenyl group, a (9-phenyl)carbazolyl group (a (9-phenyl)carbazol-1-yl group, a (9-phenyl)carbazol-2-yl group, a (9-phenyl)carbazol-3-yl group, or a (9-phenyl)carbazol-4-yl group), a (9-biphenylyl)carbazolyl group, a (9-phenyl)phenylcarbazolyl group, a diphenylcarbazol-9-yl group, a phenylcarbazol-9-yl group, a phenyltriazinyl group, a biphenylyltriazinyl group, a diphenyltriazinyl group, a phenyldibenzofuranyl group, a phenyldibenzothiophenyl group, and the like.

In this specification, the carbazolyl group is specifically each of the following groups, unless otherwise specified in this specification.

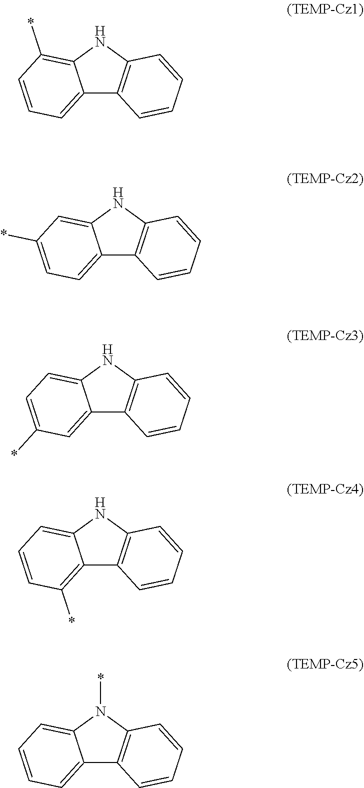 [SMILES: *c1cccc2c1[nH]c1ccccc12 ; *c1ccc2c(c1)[nH]c1ccccc12 ; *c1ccc2[nH]c3ccccc3c2c1 ; *c1cccc2[nH]c3ccccc3c12 ; *n1c2ccccc2c2ccccc21]

In this specification, the (9-phenyl)carbazolyl group is specifically any of the following groups, unless otherwise specified in this specification.

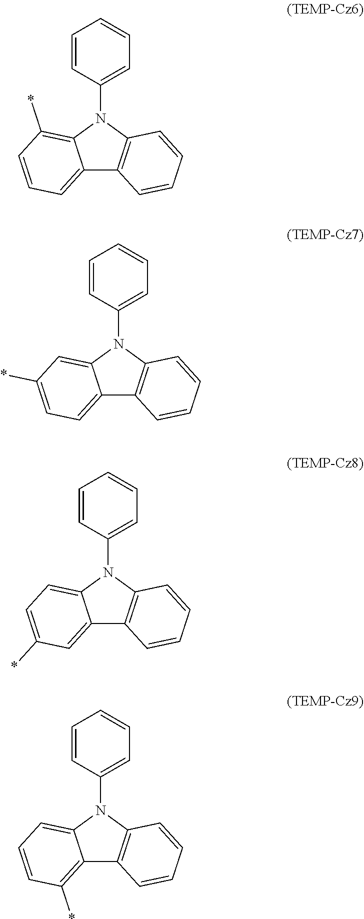 [SMILES: *c1cccc2c3ccccc3n(-c3ccccc3)c12 ; *c1ccc2c3ccccc3n(-c3ccccc3)c2c1 ; *c1ccc2c(c1)c1ccccc1n2-c1ccccc1 ; *c1cccc2c1c1ccccc1n2-c1ccccc1]

In the general formulas (TEMP-Cz1) to (TEMP-Cz9), * represents a bonding site.

In this specification, the dibenzofuranyl group and the dibenzothiophenyl group are specifically any of the following groups, unless otherwise specified in this specification.

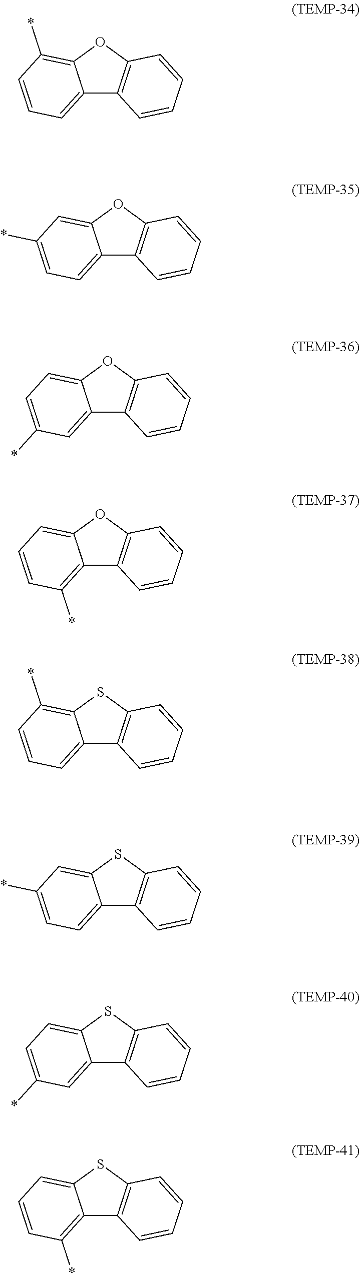

In the general formulas (TEMP-34) to (TEMP-41), * represents a bonding site.

The substituted or unsubstituted alkyl group described in this specification is preferably a methyl group, an ethyl group, a propyl group, an isopropyl group, a n-butyl group, an isobutyl group, a t-butyl group, or the like, unless otherwise specified in this specification.

“Substituted or Unsubstituted Arylene Group”

The “substituted or unsubstituted arylene group” described in this specification is a divalent group derived by removing one hydrogen atom on the aryl ring of the “substituted or unsubstituted aryl group”, unless otherwise specified. Specific examples of the “substituted or unsubstituted arylene group” (specific example group G12) include a divalent group derived by removing one hydrogen atom on the aryl ring of the “substituted or unsubstituted aryl group” described in the specific example group G1, and the like.

“Substituted or Unsubstituted Divalent Heterocyclic Group”

The “substituted or unsubstituted divalent heterocyclic group” described in this specification is a divalent group derived by removing one hydrogen atom on the heterocycle of the “substituted or unsubstituted heterocyclic group”, unless otherwise specified. Specific examples of the “substituted or unsubstituted divalent heterocyclic group” (specific example group G13) include a divalent group derived by removing one hydrogen atom on the heterocycle of the “substituted or unsubstituted heterocyclic group” described in the specific example group G2, and the like.

“Substituted or Unsubstituted Alkylene Group”

The “substituted or unsubstituted alkylene group” described in this specification is a divalent group derived by removing one hydrogen atom on the alkyl chain of the “substituted or unsubstituted alkyl group”, unless otherwise specified. Specific examples of the “substituted or unsubstituted alkylene group” (specific example group G14) include a divalent group derived by removing one hydrogen atom on the alkyl chain of the “substituted or unsubstituted alkyl group” described in the specific example group G3, and the like.

The substituted or unsubstituted arylene group described in this specification is preferably any group of the following general formulas (TEMP-42) to (TEMP-68), unless otherwise specified in this specification.

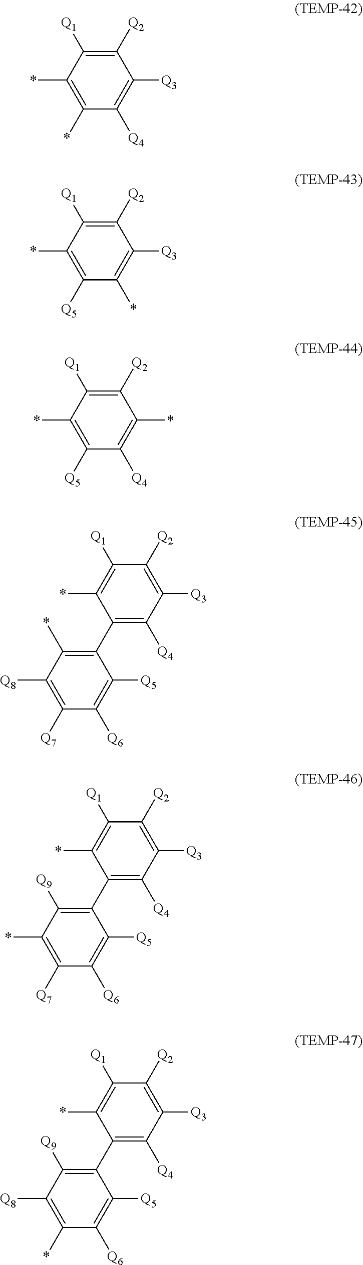

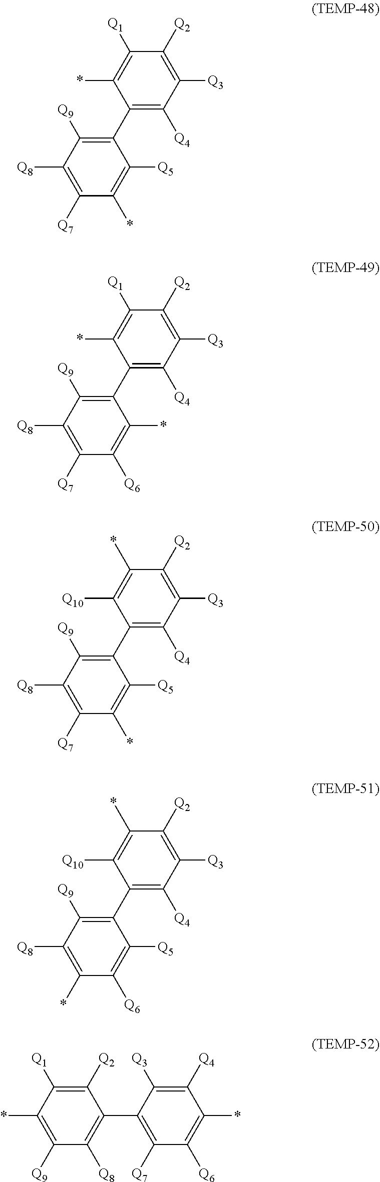

In the general formulas (TEMP-42) to (TEMP-52), Q₁ to Q₁₀ are independently a hydrogen atom or a substituent.

In the general formulas (TEMP-42) to (TEMP-52), * represents a bonding site.

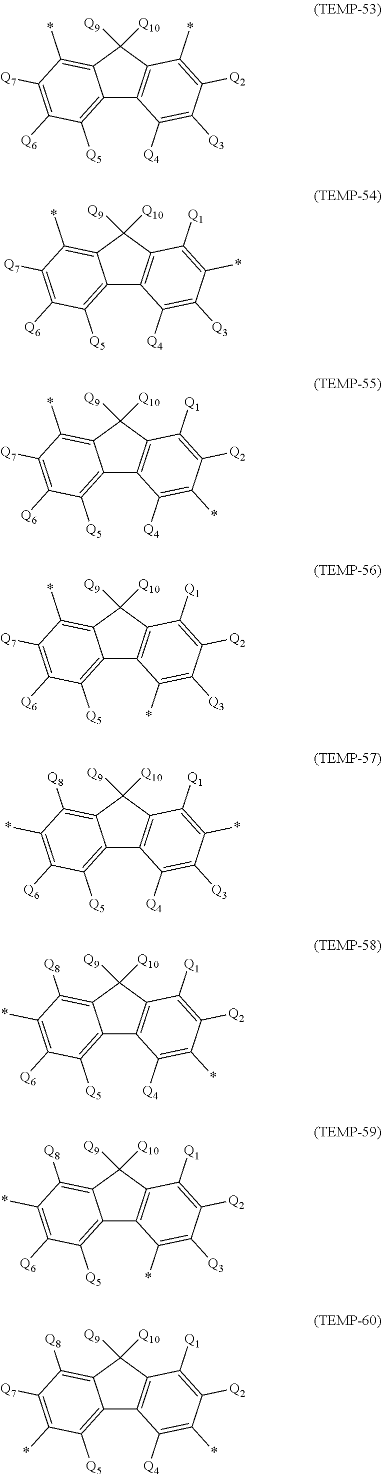

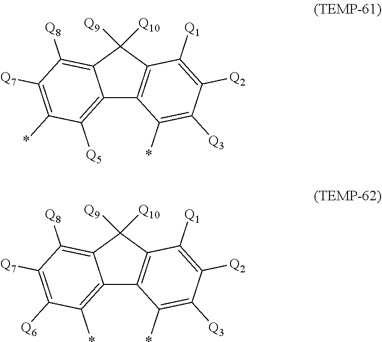

In the general formulas (TEMP-53) to (TEMP-62), Q₁ to Q₁₀ are independently a hydrogen atom or a substituent.

Q₉ and Q₁₀ may be bonded with each other via a single bond to form a ring.

In the general formulas (TEMP-53) to (TEMP-62), * represents a bonding site.

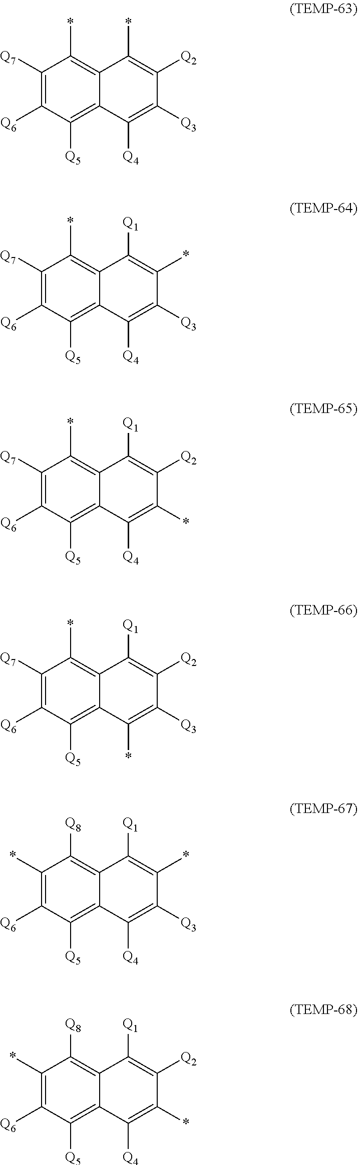

In the general formulas (TEMP-63) to (TEMP-68), Q₁ to Q₈ are independently a hydrogen atom or a substituent.

In the general formulas (TEMP-63) to (TEMP-68), * represents a bonding site.

The substituted or unsubstituted divalent heterocyclic group described in this specification is preferably any group of the following general formulas (TEMP-69) to (TEMP-102), unless otherwise specified in this specification.

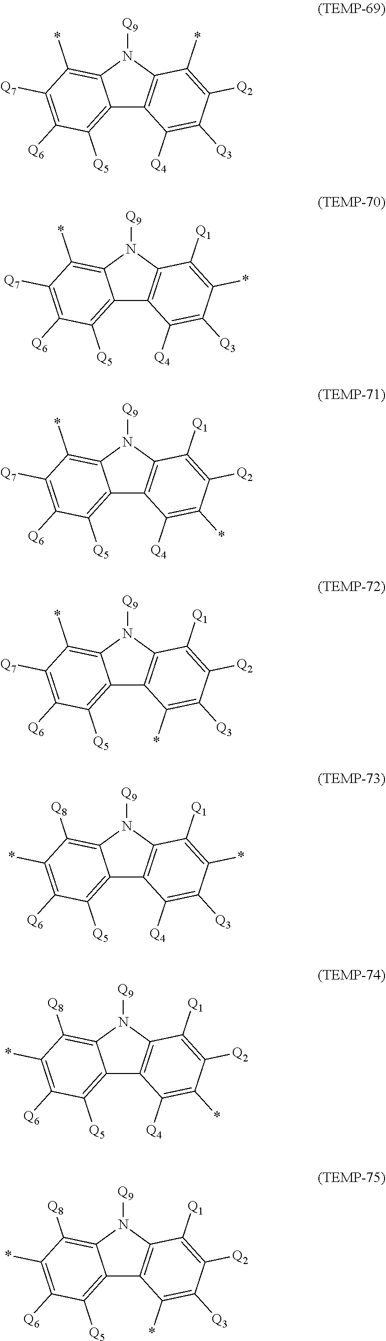

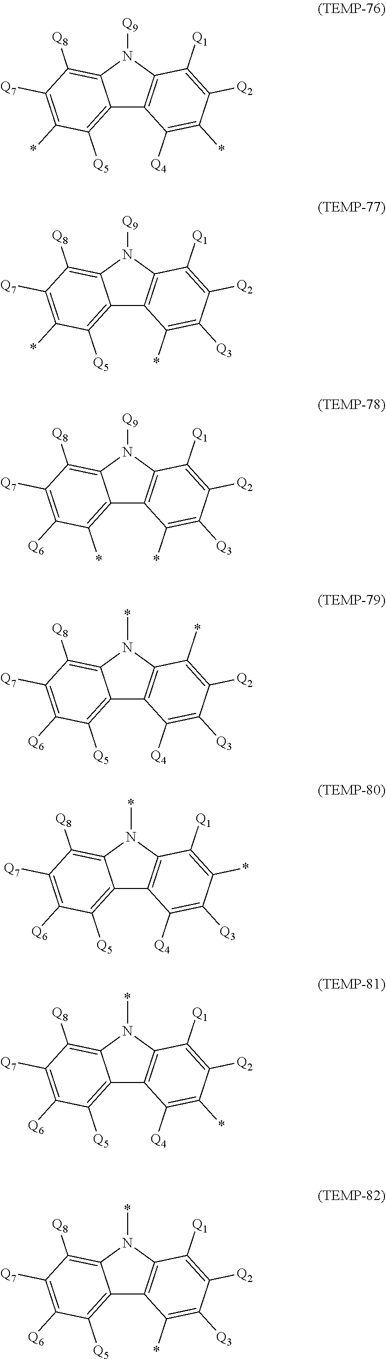

In the general formulas (TEMP-69) to (TEMP-82), Q₁ to Q₉ are independently a hydrogen atom or a substituent.

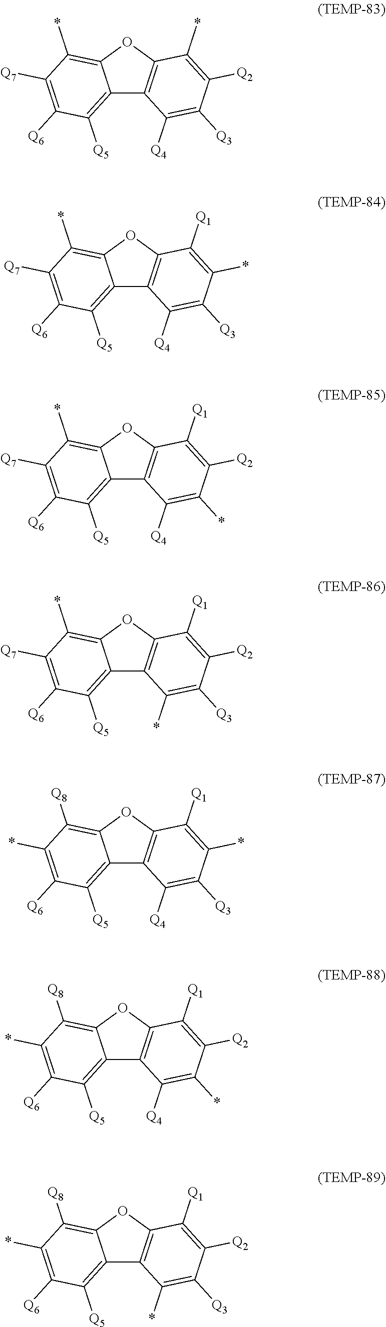

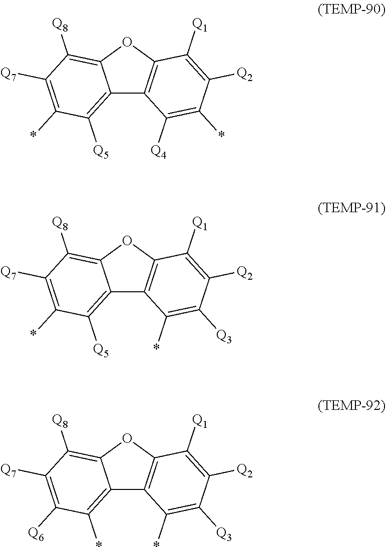

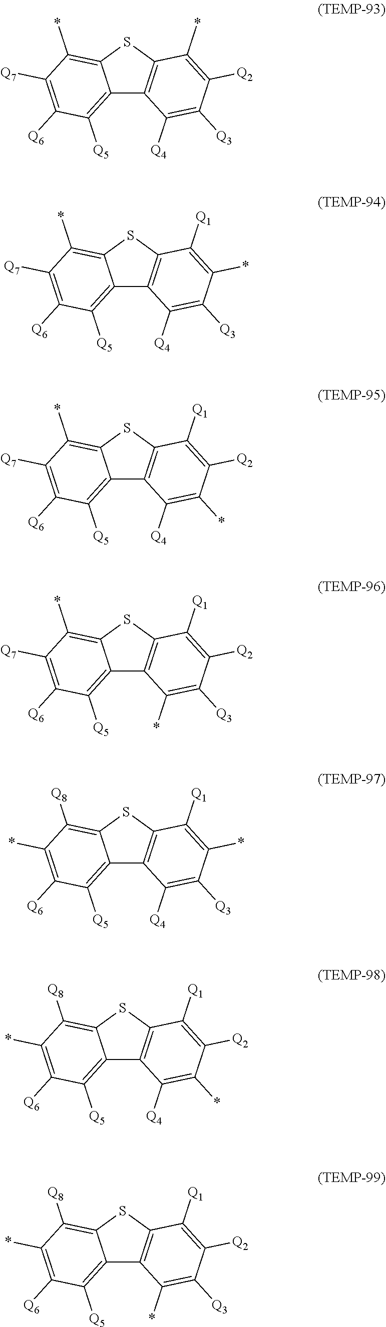

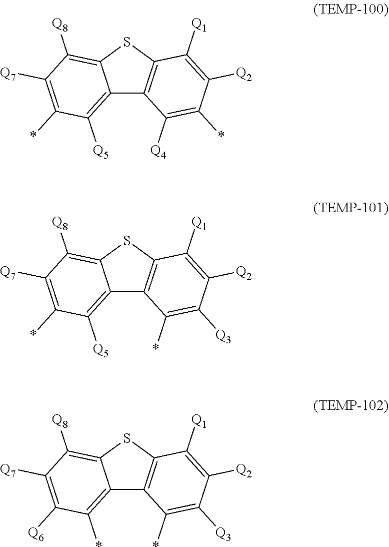

In the general formulas (TEMP-83) to (TEMP-102), Q₁ to Q₈ are independently a hydrogen atom or a substituent.

The above is the explanation of the “Substituent described in this specification.”

“The Case where Bonded with Each Other to Form a Ring”

In this specification, the case where “one or more sets of adjacent two or more form a substituted or unsubstituted monocycle by bonding with each other, form a substituted or unsubstituted fused ring by bonding with each other, or do not bond with each other” means the case where “one or more sets of adjacent two or more form a substituted or unsubstituted monocycle by bonding with each other”; the case where “one or more sets of adjacent two or more form a substituted or unsubstituted fused ring by bonding with each other”; and the case where “one or more sets of adjacent two or more do not bond with each other.”

The case where “one or more sets of adjacent two or more form a substituted or unsubstituted monocycle by bonding with each other” and the case where “one or more sets of adjacent two or more form a substituted or unsubstituted fused ring by bonding with each other” in this specification (these cases may be collectively referred to as “the case where forming a ring by bonding with each other”) will be described below. The case of an anthracene compound represented by the following general formula (TEMP-103) in which the mother skeleton is an anthracene ring will be described as an example.

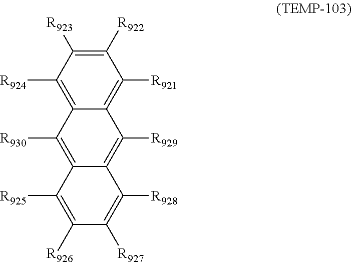

For example, in the case where “one or more sets of adjacent two or more among R₉₂₁ to R₉₃₀ form a ring by bonding with each other”, the one set of adjacent two includes a pair of R₉₂₁ and R₉₂₂, a pair of R₉₂₂ and R₉₂₃, a pair of R₉₂₃ and R₉₂₄, a pair of R₉₂₄ and R₉₃₀, a pair of R₉₃₀ and R₉₂₅, a pair of R₉₂₅ and R₉₂₆, a pair of R₉₂₆ and R₉₂₇, a pair of R₉₂₇ and R₉₂₃, a pair of R₉₂₃ and R₉₂₉, and a pair of R₉₂₉ and R₉₂₁.

The “one or more sets” means that two or more sets of the adjacent two or more sets may form a ring at the same time. For example, R₉₂₁ and R₉₂₂ forma ring Q_{A} by bonding with each other, and at the same, time R₉₂₅ and R₉₂₆ form a ring Q_{B} by bonding with each other, the anthracene compound represented by the general formula (TEMP-103) is represented by the following general formula (TEMP-104).

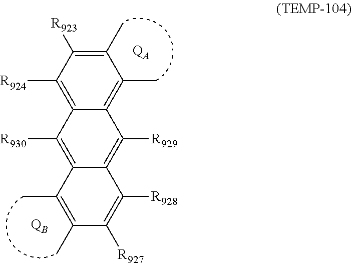

The case where the “set of adjacent two or more” form a ring includes not only the case where the set (pair) of adjacent “two” is bonded with as in the above-mentioned examples, but also the case where the set of adjacent “three or more” are bonded with each other. For example, it means the case where R₉₂₁ and R₉₂₂ form a ring Q_{A} by bonding with each other, and R₉₂₂ and R₉₂₃ form a ring Q_{C} by bonding with each other, and adjacent three (R₉₂₁, R₉₂₂ and R₉₂₃) form rings by bonding with each other and together fused to the anthracene mother skeleton. In this case, the anthracene compound represented by the general formula (TEMP-103) is represented by the following general formula (TEMP-105). In the following general formula (TEMP-105), the ring Q_{A} and the ring Q_{C} share R₉₂₂.

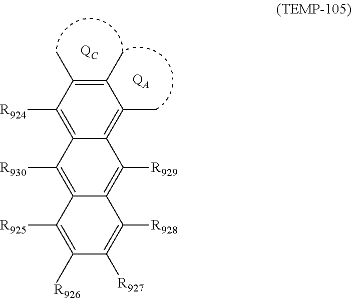

The “monocycle” or “fused ring” formed may be a saturated ring or an unsaturated ring, as a structure of the formed ring alone. Even when the “one pair of adjacent two” forms a “monocycle” or a “fused ring”, the “monocycle” or the “fused ring” may forma saturated ring or an unsaturated ring. For example, the ring Q_{A} and the ring Q_{B} formed in the general formula (TEMP-104) are independently a “monocycle” or a “fused ring.” The ring Q_{A} and the ring Q_{C} formed in the general formula (TEMP-105) are “fused ring.” The ring Q_{A} and ring Q_{C} of the general formula (TEMP-105) are fused ring by fusing the ring Q_{A} and the ring Q_{C} together. When the ring Q_{A} of the general formula (TEMP-104) is a benzene ring, the ring Q_{A} is a monocycle. When the ring Q_{A} of the general formula (TEMP-104) is a naphthalene ring, the ring Q_{A} is a fused ring.

The “unsaturated ring” includes, in addition to an aromatic hydrocarbon ring and an aromatic heterocycle, an aliphatic hydrocarbon ring with an unsaturated bond, i.e., double and/or triple bonds in the ring structure (e.g., cyclohexene, cyclohexadiene, etc.), and a non-aromatic heterocycle with an unsaturated bond (e.g., dihydropyran, imidazoline, pyrazoline, quinolizine, indoline, isoindoline, etc.). The “saturated ring” includes an aliphatic hydrocarbon ring without an unsaturated bond and a non-aromatic heterocycle without ab unsaturated bond.

Specific examples of the aromatic hydrocarbon ring include a structure in which the group listed as a specific example in the specific example group G1 is terminated by a hydrogen atom.

Specific examples of the aromatic heterocycle include a structure in which the aromatic heterocyclic group listed as a specific example in the example group G2 is terminated by a hydrogen atom.

Specific examples of the aliphatic hydrocarbon ring include a structure in which the group listed as a specific example in the specific example group G6 is terminated by a hydrogen atom.

The term “to form a ring” means forming a ring only with plural atoms of the mother skeleton, or with plural atoms of the mother skeleton and one or more arbitrary atoms in addition. For example, the ring Q_{A} shown in the general formula (TEMP-104), which is formed by bonding R₉₂₁ and R₉₂₂ with each other, is a ring formed from the carbon atom of the anthracene skeleton with which R₉₂₁ is bonded, the carbon atom of the anthracene skeleton with which R₉₂₂ is bonded, and one or more arbitrary atoms. For example, in the case where the ring Q_{A} is formed with R₉₂₁ and R₉₂₂, when a monocyclic unsaturated ring is formed with the carbon atom of the anthracene skeleton with which R₉₂₁ is bonded, the carbon atom of the anthracene skeleton with which R₉₂₂ is bonded, and four carbon atoms, the ring formed with R₉₂₁ and R₉₂₂ is a benzene ring.

Here, the “arbitrary atom” is preferably at least one atom selected from the group consisting of a carbon atom, a nitrogen atom, an oxygen atom, and a sulfur atom, unless otherwise specified in this specification. In the arbitrary atom (for example, a carbon atom or a nitrogen atom), a bond which does not form a ring may be terminated with a hydrogen atom or the like, or may be substituted with “arbitrary substituent” described below. When an arbitrary atom other than a carbon atom is contained, the ring formed is a heterocycle.

The number of “one or more arbitrary atom(s)” constituting a monocycle or a fused ring is preferably 2 or more and 15 or less, more preferably 3 or more and 12 or less, and still more preferably 3 or more and 5 or less, unless otherwise specified in this specification.

The “monocycle” is preferable among the “monocycle” and the “fused ring”, unless otherwise specified in this specification.

The “unsaturated ring” is preferable among the “saturated ring” and the “unsaturated ring”, unless otherwise specified in this specification.

Unless otherwise specified in this specification, the “monocycle” is preferably a benzene ring.

Unless otherwise specified in this specification, the “unsaturated ring” is preferably a benzene ring.

Unless otherwise specified in this specification, when “one or more sets of adjacent two or more” are “bonded with each other to form a substituted or unsubstituted monocycle” or “bonded with each other to form a substituted or unsubstituted fused ring”, this specification, one or more sets of adjacent two or more are preferably bonded with each other to form a substituted or unsubstituted “unsaturated ring” from plural atoms of the mother skeleton and one or more and 15 or less atoms which is at least one kind selected from a carbon atom, a nitrogen atom, an oxygen atom, and a sulfur atom.

The substituent in the case where the above-mentioned “monocycle” or “fused ring” has a substituent is, for example, an “arbitrary substituent” described below. Specific examples of the substituent which the above-mentioned “monocycle” or “fused ring” has include the substituent described above in the “Substituent described in this specification” section.

The substituent in the case where the above-mentioned “saturated ring” or “unsaturated ring” has a substituent is, for example, an “arbitrary substituent” described below. Specific examples of the substituent which the above-mentioned “monocycle” or “fused ring” has include the substituent described above in the “Substituent described in this specification” section.

The foregoing describes the case where “one or more sets of adjacent two or more form a substituted or unsubstituted monocycle by bonding with each other” and the case where “one or more sets of adjacent two or more form a substituted or unsubstituted fused ring by bonding with each other” (the case where “forming a ring by bonding with each other”).

Substituent in the Case of “Substituted or Unsubstituted”

In one embodiment in this specification, the substituent (in this specification, sometimes referred to as an “arbitrary substituent”) in the case of “substituted or unsubstituted” is, for example, a group selected from the group consisting of:

an unsubstituted alkyl group including 1 to 50 carbon atoms,

an unsubstituted alkenyl group including 2 to 50 carbon atoms,

an unsubstituted alkynyl group including 2 to 50 carbon atoms,

an unsubstituted cycloalkyl group including 3 to 50 ring carbon atoms,

—Si(R₉₀₁)(R₉₀₂)(R₉₀₃),

—O—(R₉₀₄),

—S—(R₉₀₅),

—N(R₉₀₆)(R₉₀₇),

a halogen atom, a cyano group, a nitro group,

an unsubstituted aryl group including 6 to 50 ring carbon atoms, and

an unsubstituted heterocyclic group including 5 to 50 ring atoms,

wherein, R₉₀₁ to R₉₀₇ are independently

a hydrogen atom,

a substituted or unsubstituted alkyl group including 1 to 50 carbon atoms,

a substituted or unsubstituted cycloalkyl group including 3 to 50 ring carbon atoms,

a substituted or unsubstituted aryl group including 6 to 50 ring carbon atoms, or

a substituted or unsubstituted heterocyclic group including 5 to 50 ring atoms.

When two or more R₉₀₁'s are present, the two or more R₉₀₁'s may be the same or different.

When two or more R₉₀₂'s are present, the two or more R₉₀₂'s may be the same or different.

When two or more R₉₀₃'s are present, the two or more R₉₀₃'s may be the same or different.

When two or more R₉₀₄'s are present, the two or more R₉₀₄'s may be the same or different.

When two or more R₉₀₅'s are present, the two or more R₉₀₅'s may be the same or different.

When two or more R₉₀₆'s are present, the two or more R₉₀₆'s may be the same or different.

When two or more R₉₀₇'s are present, the two or more R₉₀₇'s may be the same or different.

In one embodiment, the substituent in the case of “substituted or unsubstituted” is a group selected from the group consisting of:

an alkyl group including 1 to 50 carbon atoms,

an aryl group including 6 to 50 ring carbon atoms, and

a heterocyclic group including 5 to 50 ring atoms.

In one embodiment, the substituent in the case of “substituted or unsubstituted” is a group selected from the group consisting of:

an alkyl group including 1 to 18 carbon atoms,

an aryl group including 6 to 18 ring carbon atoms, and

a heterocyclic group including 5 to 18 ring atoms.

Specific examples of each of the arbitrary substituents include specific examples of substituent described in the section “Substituent described in this specification” above.

Unless otherwise specified in this specification, adjacent arbitrary substituents may form a “saturated ring” or an “unsaturated ring”, preferably form a substituted or unsubstituted saturated 5-membered ring, a substituted or unsubstituted saturated 6-membered ring, a substituted or unsubstituted unsaturated 5-membered ring, or a substituted or unsubstituted unsaturated 6-membered ring, more preferably form a benzene ring.

Unless otherwise specified in this specification, the arbitrary substituent may further have a substituent. The substituent which the arbitrary substituent further has is the same as that of the above-mentioned arbitrary substituent.

In this specification, the numerical range represented by “AA to BB” means the range including the numerical value AA described on the front side of “AA to BB” as the lower limit and the numerical value BB described on the rear side of “AA to BB” as the upper limit.

### [Novel Compound]

The compound according to an aspect of the invention is represented by the following formula (1).

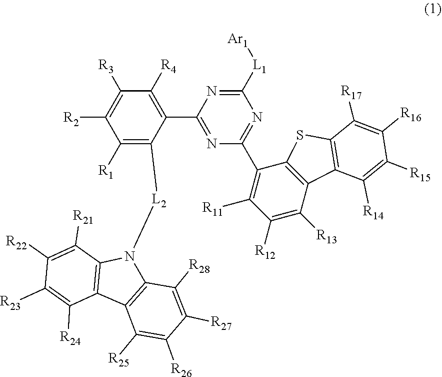

wherein in the formula (1),

Ar₁ is a substituted or unsubstituted aryl group including 6 to 50 ring carbon atoms;

L₁ and L₂ are independently a single bond or a substituted or unsubstituted arylene group including 6 to 50 ring carbon atoms;

each of R₁ to R₄ is a hydrogen atom, a substituted or unsubstituted alkyl group including 1 to 50 carbon atoms, or a substituted or unsubstituted aryl group including 6 to 50 ring carbon atoms; one or more sets of adjacent two or more of R₁ to R₄ are not bonded with each other;

one or more sets of adjacent two or more of R₁₁ to R₁₇ form a substituted or unsubstituted, saturated or unsaturated ring by bonding with each other, or do not form a substituted or unsubstituted, saturated or unsaturated ring; R₁₁ to R₁₇ which do not form the substituted or unsubstituted, saturated or unsaturated ring are independently a hydrogen atom or a substituent R;

R₂₁ and R₂₂ form a substituted or unsubstituted, saturated or unsaturated ring by bonding with each other, or do not form a substituted or unsubstituted, saturated or unsaturated ring; one or more sets of adjacent two or more of R₂₃ to R₂₆ form a substituted or unsubstituted, saturated or unsaturated ring by bonding with each other, or do not form a substituted or unsubstituted, saturated or unsaturated ring; R₂₇ and R₂₈ form a substituted or unsubstituted, saturated or unsaturated ring by bonding with each other, or do not form a substituted or unsubstituted, saturated or unsaturated ring; R₂₂ and R₂₃ are not bonded with each other;

R₂₆ and R₂₇ are not bonded with each other;

R₂₁ to R₂₈ which do not form a substituted or unsubstituted, saturated or unsaturated ring are independently a hydrogen atom or a substituent R;

the substituent R is selected from the group consisting of

a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),

### —O—(R₉₀₄),

### —S—(R₉₀₅),

—N(R₉₀₆)(R₉₀₇)
(where R₉₀₁ to R₉₀₇ are independently
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms; and when two or more
of each of R₉₀₁ to R₉₀₇ are present, the two or more of each of R₉₀₁ to R₉₀₇ may be the same as or different from each other),
a halogen atom, a cyano group, a nitro group,
a substituted or unsubstituted aryl group including 6 to 50 ring carbon atoms, and
a substituted or unsubstituted monovalent heterocyclic group including 5 to 50 ring atoms; and

when two or more substituent R's are present, the two or more substituent R's may be the same as or different from each other.

The compound according to an aspect of the invention having the above structure, when used for an organic EL device can increase its device performance As one of the specific effects, the compound can realize an organic EL device having a long lifetime.

The compound according to an aspect of the invention has a structure in which a 4-dibenzothiophenyl group is directly bonded with a triazine ring as shown in the formula (1). Since the triazine ring has the absolute value of the affinity larger than that of the pyrimidine ring which is the same nitrogen-containing 6-membered ring, for example (i.e. the affinity level is deeper). Among 1- to 8-position of a dibenzothiophenyl group, one having a bond at a 4-position (or 5-position) has the largest absolute value of the affinity. Therefore, it is considered that the compound according to an aspect of the invention has an excellent electron-injecting property from the cathode and can contribute to increase of the performance of the organic EL device. In addition, the compound has the phenylene group (i.e. the phenylene group with which R₁ to R₄ are bonded) directly connected to the triazine ring at its ortho position, thereby the electron mobility can be controlled and the lifetime of the organic EL device can be increased.

In one embodiment, Ar₁ is a group represented by the following formula (1A):

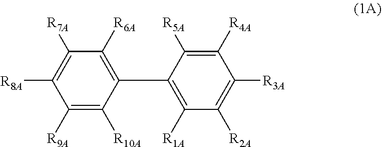

wherein in the formula (1A),

one of R_{1A} to R_{10A} represents a bond which bonds with L₁ in the formula (1);

adjacent two or more of R_{1A} to R_{10A} which do not represent the bond which bonds with L₁ form a substituted or unsubstituted, saturated or unsaturated ring by bonding with each other, or do not form a substituted or unsubstituted, saturated or unsaturated ring;

R_{1A} to R_{10A} which do not represent the bond which bonds with L₁ and do not form the substituted or unsubstituted, saturated or unsaturated ring are independently a hydrogen atom or a substituent R; and

the substituent R is as defined in the formula (1).

One of R_{1A} to R_{10A} represents a bond which bonds with L₁ in the formula (1). The expression “represent a bond” means that L₁ is directly bonded with one of the carbon atoms on the benzene ring with which R_{1A} to R_{10A} is bonded. In the case when L₁ is a single bond, the carbon atom on the triazine ring with which L₁ is bonded and one of the carbon atoms on the benzene ring with which R_{1A} to R_{10A} is bonded are directly bonded through a single bond.

In one embodiment, R_{1A} in the formula (1A) represents a bond which bonds with L₁.

In one embodiment, R_{5A} and R_{6A} in the formula (1A) form a substituted or unsubstituted, saturated or unsaturated ring by bonding with each other.

In one embodiment, R_{1A} to R_{10A} which do not represent the bond which bonds with L₁ and do not form the substituted or unsubstituted, saturated or unsaturated ring are independently a hydrogen atom, a substituted or unsubstituted alkyl group including 1 to 50 carbon atoms, or a substituted or unsubstituted aryl group including 6 to 50 ring carbon atoms.

In one embodiment, L₁ is a single bond.

In one embodiment, the compound represented by the formula (1) is a compound represented by the following formula (1-1):

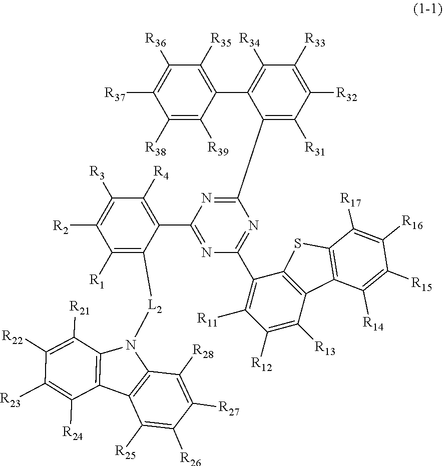

wherein in the formula (1-1),

L₂, R₁ to R₄, R₁₁ to R₁₇, and R₂₁ to R₂₈ are as defined in the formula (1);

one or more sets of adjacent two or more of R₃₁ to R₃₉ form a substituted or unsubstituted, saturated or unsaturated ring by bonding with each other, or do not form a substituted or unsubstituted, saturated or unsaturated ring;

R₃₁ to R₃₉ which do not form the substituted or unsubstituted, saturated or unsaturated ring are independently a hydrogen atom or a substituent R; and

the substituent R is as defined in the formula (1).

In one embodiment, R₃₄ and R₃₅ in the formula (1-1) form a substituted or unsubstituted, saturated or unsaturated ring by bonding with each other.

In one embodiment, R₃₁ to R₃₉ which do not form the substituted or unsubstituted, saturated or unsaturated ring are independently a hydrogen atom, a substituted or unsubstituted alkyl group including 1 to 50 carbon atoms, or a substituted or unsubstituted aryl group including 6 to 50 ring carbon atoms,

In one embodiment, R₁ to R₄ are hydrogen atoms.

In one embodiment, sets of adjacent two or more of R₁₁ to R₁₇ are not bonded with each other, sets of adjacent two or more of R₃₁ to R₃₉ are not bonded with each other, and sets of adjacent two or more of R₂₁ to R₂₈ are not bonded with each other.

In one embodiment, sets of adjacent two or more of R₁₁ to R₁₇ are not bonded with each other, and R₁₁ to R₁₇ are a hydrogen atom, a substituted or unsubstituted alkyl group including 1 to 50 carbon atoms, or a substituted or unsubstituted aryl group including 6 to 50 ring carbon atoms.

In one embodiment, sets of adjacent two or more of R₁₁ to R₁₇ are not bonded with each other and R₁₁ to R₁₇ are hydrogen atoms.

In one embodiment, the compound represented by the formula (1-1) is a compound represented by the following formula (1-11):

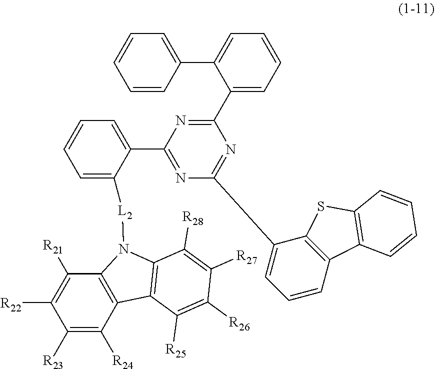

wherein in the formula (1-11), L₂ and R₂₁ to R₂₈ are as defined in the formula (1-1).

In one embodiment, the compound represented by the formula (1-1) is a compound represented by the following formula (1-12):

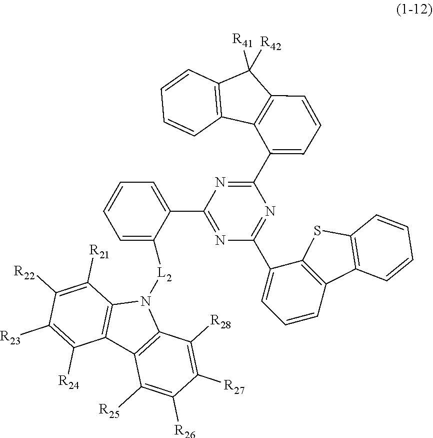

wherein in the formula (1-12), L₂ and R₂₁ to R₂₈ are as defined in the formula (1-1);

R₄₁ and R₄₂ are independently a hydrogen atom or a substituent R; and the substituent R is as defined in the formula (1).

In one embodiment, R₄₁ and R₄₂ are independently a substituted or unsubstituted alkyl group including 1 to 50 carbon atoms, or a substituted or unsubstituted aryl group including 6 to 18 ring carbon atoms.

In one embodiment, one or more sets of adjacent two or more of R₂₁ to R₂₈ are not bonded with each other.

In one embodiment, adjacent two or more of R₂₁ to R₂₈ are not bonded with each other, and R₂₁ to R₂₈ are independently a hydrogen atom, a substituted or unsubstituted alkyl group including 1 to 50 carbon atoms, or a substituted or unsubstituted aryl group including 6 to 18 ring carbon atoms.

In one embodiment, sets of adjacent two or more of R₂₁ to R₂₈ are not bonded with each other, and R₂₁ to R₂₈ are hydrogen atoms.

In one embodiment, L₂ is a single bond.

In one embodiment, L₂ is a substituted or unsubstituted arylene group including 6 to 18 ring carbon atoms.

In one embodiment, sets of adjacent two or more of R₁₁ to R₁₇ are not bonded with each other, and sets of adjacent two or more of R₂₁ to R₂₃ are not bonded with each other.

As described in [Definition], a term “hydrogen atom” used in this specification includes a protium atom, a deuterium atom, and a tritium atom. Accordingly, the compound according to the invention may have a naturally derived deuterium atom.

Further, a deuterium atom may be intentionally introduced into the compound according to the invention by replacing part or all of the raw material compounds with their deuterated compounds. Accordingly, in one embodiment of the invention, the compound represented by the formula (1) has at least one deuterium atom. In other words, the compound of this embodiment may be a compound represented by the formula (1), wherein at least one of hydrogen atoms possessed by the compound is a deuterium atom.

In the compound represented by the formula (1), at least one hydrogen atom selected from hydrogen atoms possessed by Ar₁; hydrogen atoms possessed by L₁; hydrogen atoms when R₁ to R₄ are hydrogen atoms; hydrogen atoms when R₁₁ to R₁₇ are hydrogen atoms; hydrogen atoms possessed by L₂; and hydrogen atoms when R₂₁ to R₂₈ are hydrogen atoms may be a deuterium atom.

The deuteration rate of a compound depends on the deuteration rate of its raw material compounds used. Even if a raw material having a predetermined deuteration rate is used, it may be contain a naturally derived protium isotope therein at a certain ratio. Therefore, the deuteration rate is obtained by taking a ratio of small amount of the naturally derived isotope into consideration, relative to the ratio obtained by merely counting the number of deuterium atoms in the chemical formula.

In one embodiment, the deuteration rate of the compound is, for example, 1% or more, 3% or more, 5% or more, 10% or more, or 50% or more.

In one embodiment, the substituent in the case of the “substituted or unsubstituted” and the substituent R are groups selected from the group consisting of

an alkyl group including 1 to 50 carbon atoms,
an aryl group including 6 to 50 ring carbon atoms, and
a heterocyclic group including 5 to 50 ring atoms.

In one embodiment, the substituent in the case of the “substituted or unsubstituted” and the substituent R are groups selected from the group consisting of

an alkyl group including 1 to 18 carbon atoms,
an aryl group including 6 to 18 ring carbon atoms, and
a heterocyclic group including 5 to 18 ring atoms.

Specific examples of the compound represented by the formula (1) will be described below, but these are merely examples, and the compound represented by the formula (1) is not limited to the following specific examples.

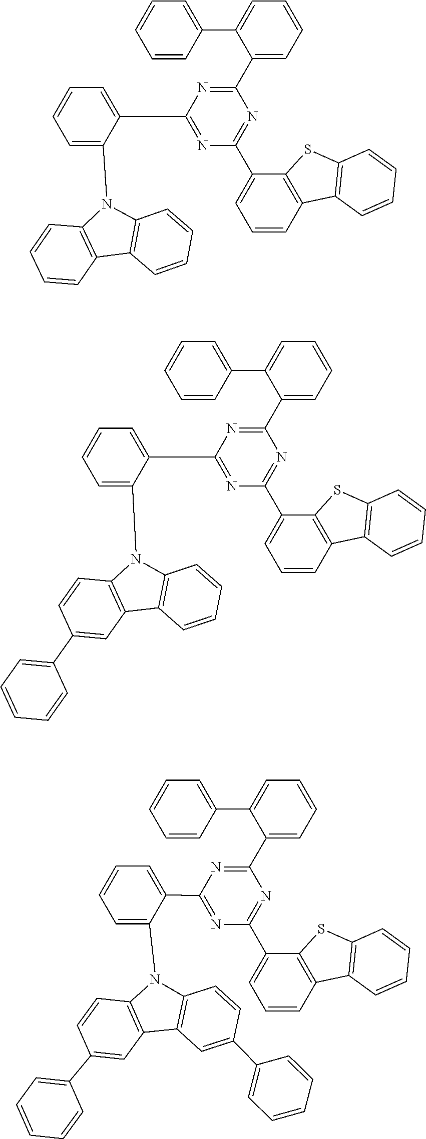 [SMILES: c1ccc(-c2ccccc2-c2nc(-c3ccccc3-n3c4ccccc4c4ccccc43)nc(-c3cccc4c3sc3ccccc34)n2)cc1 ; c1ccc(-c2ccc3c(c2)c2ccccc2n3-c2ccccc2-c2nc(-c3ccccc3-c3ccccc3)nc(-c3cccc4c3sc3ccccc34)n2)cc1 ; c1ccc(-c2ccc3c(c2)c2cc(-c4ccccc4)ccc2n3-c2ccccc2-c2nc(-c3ccccc3-c3ccccc3)nc(-c3cccc4c3sc3ccccc34)n2)cc1]

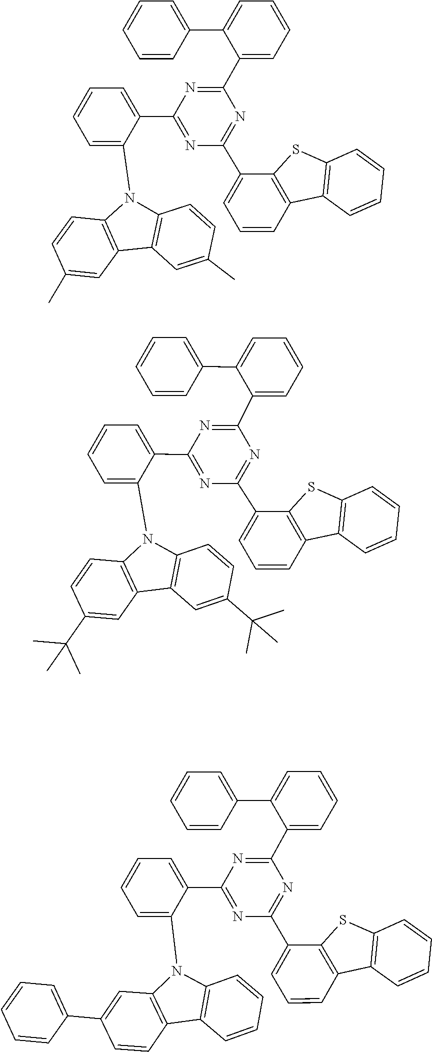 [SMILES: Cc1ccc2c(c1)c1cc(C)ccc1n2-c1ccccc1-c1nc(-c2ccccc2-c2ccccc2)nc(-c2cccc3c2sc2ccccc23)n1 ; CC(C)(C)c1ccc2c(c1)c1cc(C(C)(C)C)ccc1n2-c1ccccc1-c1nc(-c2ccccc2-c2ccccc2)nc(-c2cccc3c2sc2ccccc23)n1 ; c1ccc(-c2ccc3c4ccccc4n(-c4ccccc4-c4nc(-c5ccccc5-c5ccccc5)nc(-c5cccc6c5sc5ccccc56)n4)c3c2)cc1]

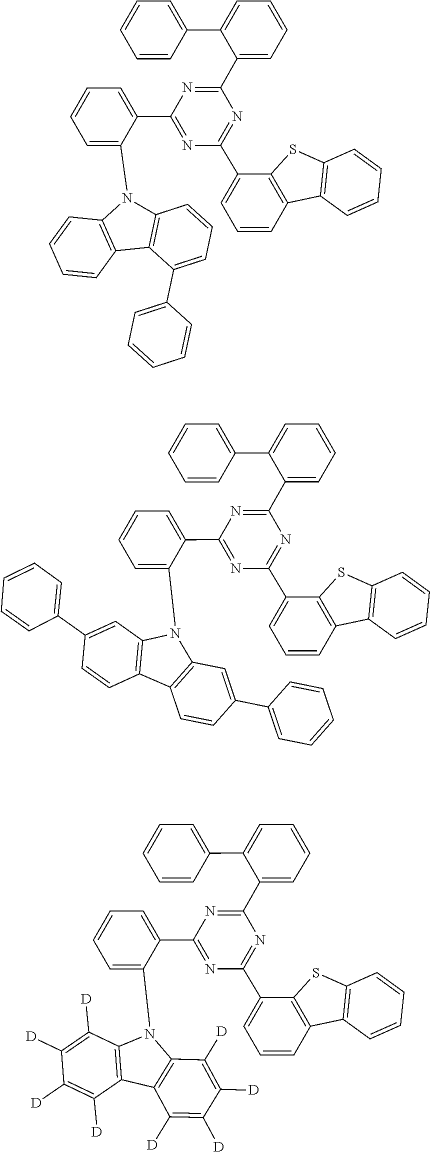 [SMILES: c1ccc(-c2ccccc2-c2nc(-c3ccccc3-n3c4ccccc4c4c(-c5ccccc5)cccc43)nc(-c3cccc4c3sc3ccccc34)n2)cc1 ; c1ccc(-c2ccc3c4ccc(-c5ccccc5)cc4n(-c4ccccc4-c4nc(-c5ccccc5-c5ccccc5)nc(-c5cccc6c5sc5ccccc56)n4)c3c2)cc1 ; [2H]c1c([2H])c([2H])c2c(c1[2H])c1c([2H])c([2H])c([2H])c([2H])c1n2-c1ccccc1-c1nc(-c2ccccc2-c2ccccc2)nc(-c2cccc3c2sc2ccccc23)n1]

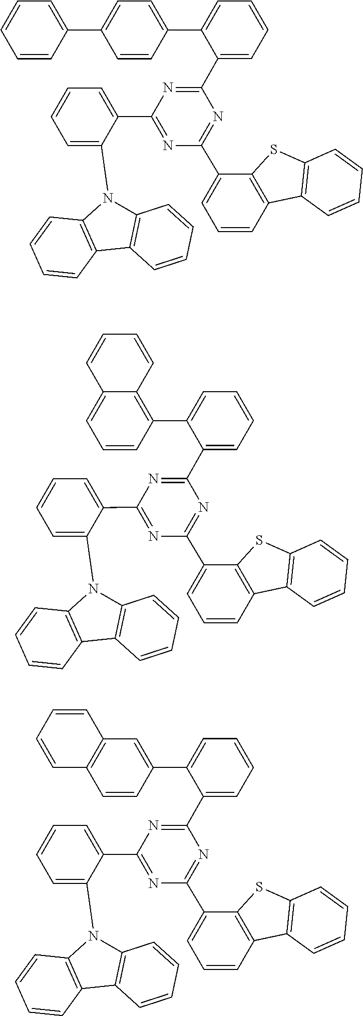

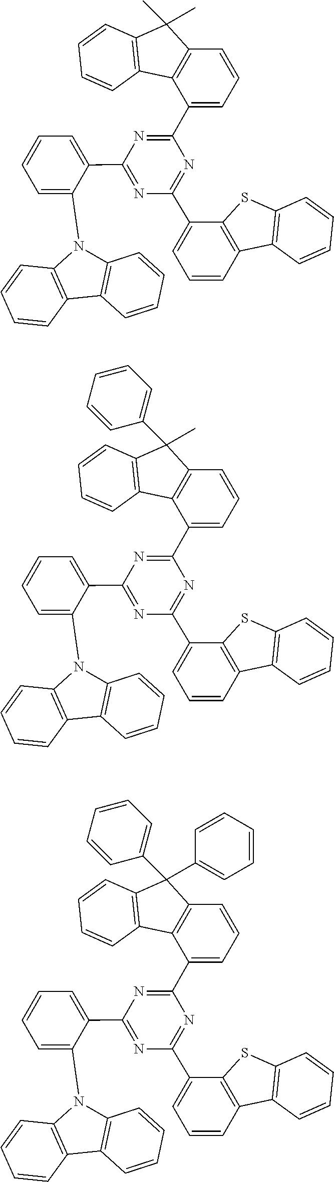 [SMILES: CC1(C)c2ccccc2-c2c(-c3nc(-c4ccccc4-n4c5ccccc5c5ccccc54)nc(-c4cccc5c4sc4ccccc45)n3)cccc21 ; CC1(c2ccccc2)c2ccccc2-c2c(-c3nc(-c4ccccc4-n4c5ccccc5c5ccccc54)nc(-c4cccc5c4sc4ccccc45)n3)cccc21 ; c1ccc(C2(c3ccccc3)c3ccccc3-c3c(-c4nc(-c5ccccc5-n5c6ccccc6c6ccccc65)nc(-c5cccc6c5sc5ccccc56)n4)cccc32)cc1]

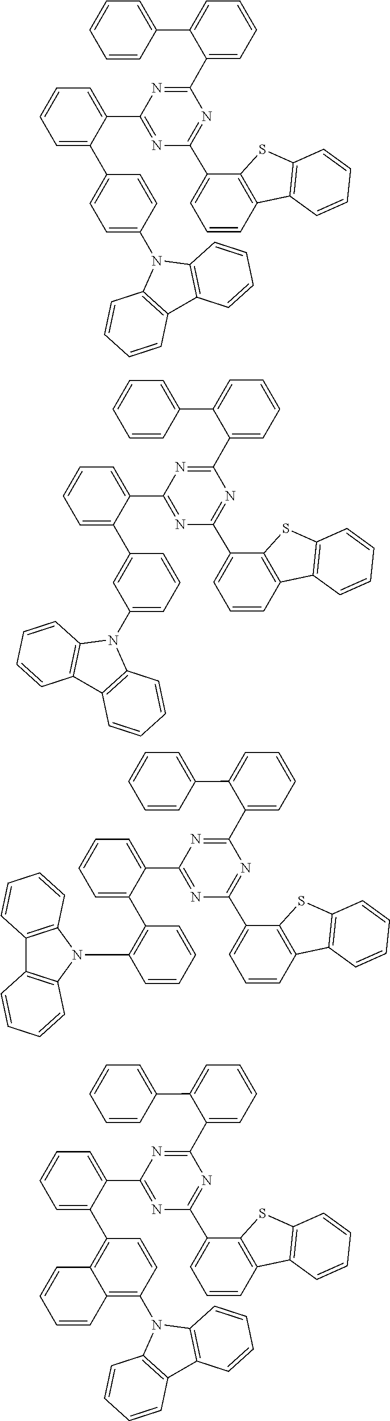

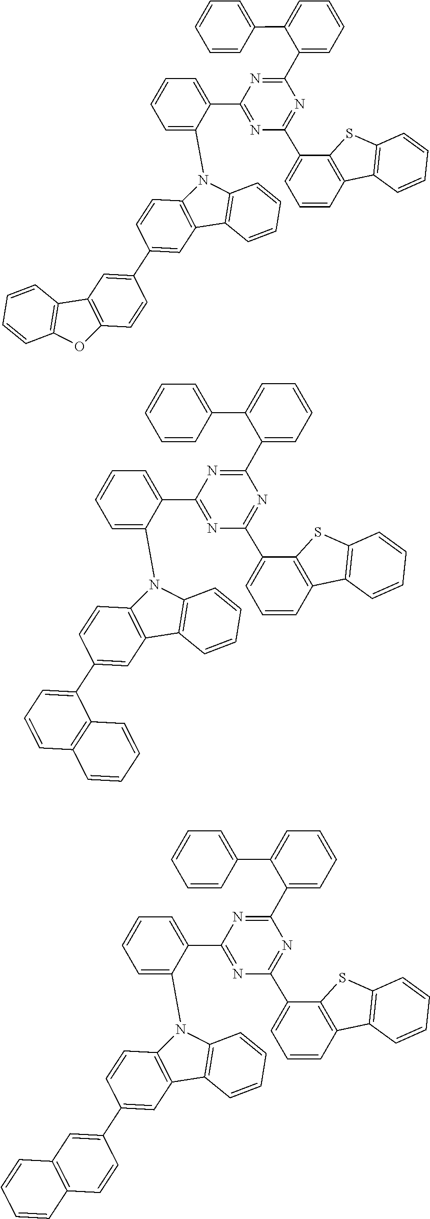 [SMILES: c1ccc(-c2ccccc2-c2nc(-c3ccccc3-n3c4ccccc4c4cc(-c5ccc6oc7ccccc7c6c5)ccc43)nc(-c3cccc4c3sc3ccccc34)n2)cc1 ; c1ccc(-c2ccccc2-c2nc(-c3ccccc3-n3c4ccccc4c4cc(-c5cccc6ccccc56)ccc43)nc(-c3cccc4c3sc3ccccc34)n2)cc1 ; c1ccc(-c2ccccc2-c2nc(-c3ccccc3-n3c4ccccc4c4cc(-c5ccc6ccccc6c5)ccc43)nc(-c3cccc4c3sc3ccccc34)n2)cc1]

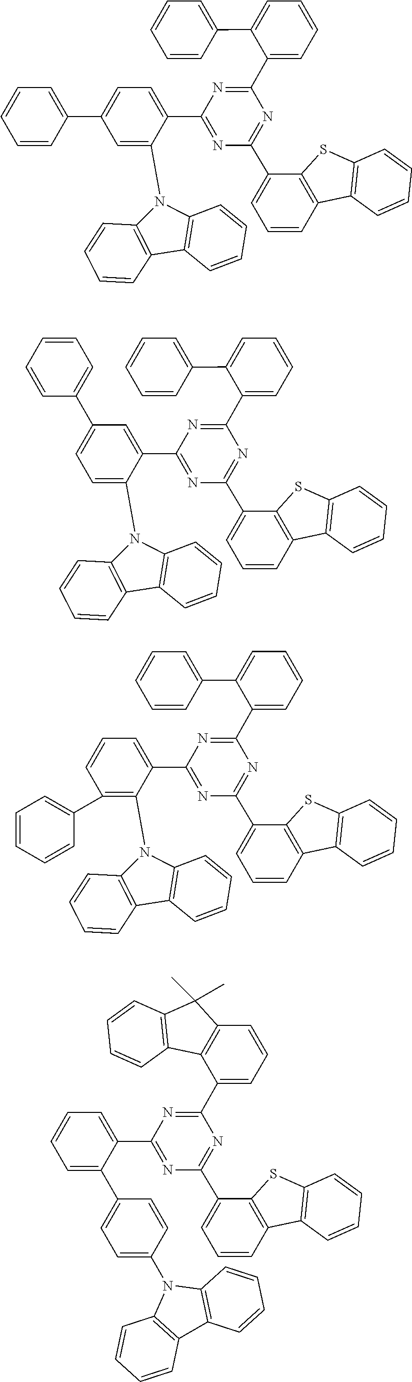

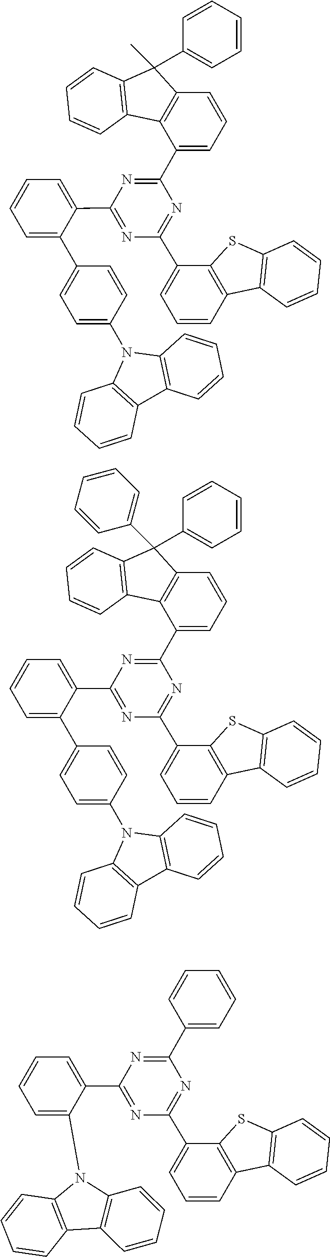

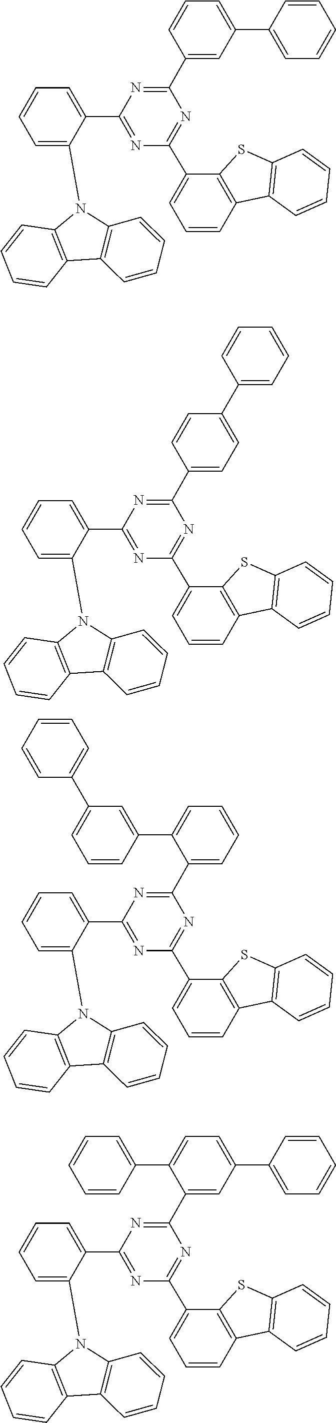

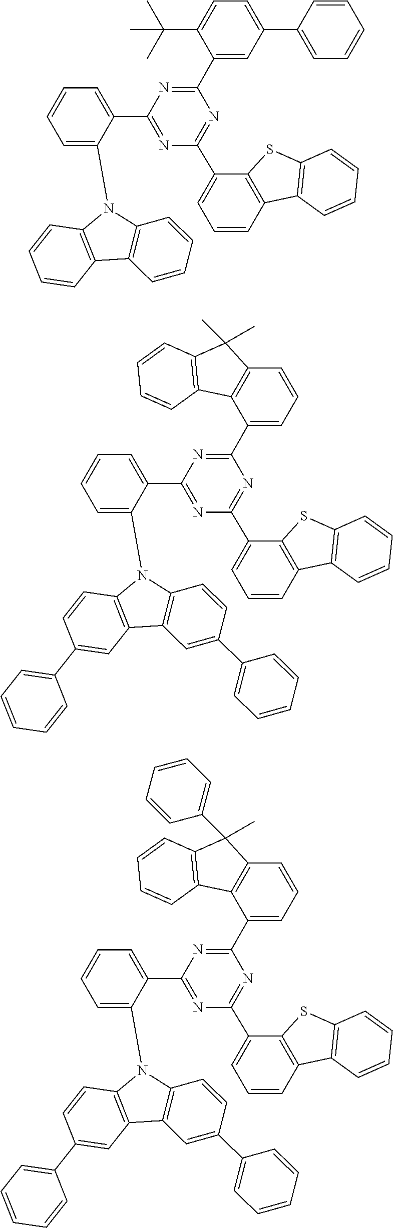 [SMILES: CC(C)(C)c1ccc(-c2ccccc2)cc1-c1nc(-c2ccccc2-n2c3ccccc3c3ccccc32)nc(-c2cccc3c2sc2ccccc23)n1 ; CC1(C)c2ccccc2-c2c(-c3nc(-c4ccccc4-n4c5ccc(-c6ccccc6)cc5c5cc(-c6ccccc6)ccc54)nc(-c4cccc5c4sc4ccccc45)n3)cccc21 ; CC1(c2ccccc2)c2ccccc2-c2c(-c3nc(-c4ccccc4-n4c5ccc(-c6ccccc6)cc5c5cc(-c6ccccc6)ccc54)nc(-c4cccc5c4sc4ccccc45)n3)cccc21]

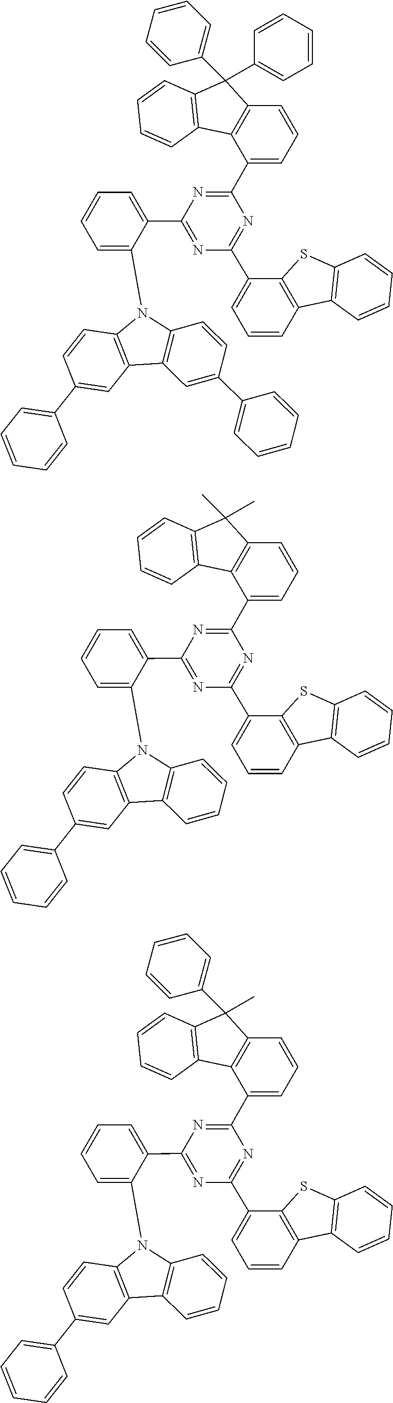

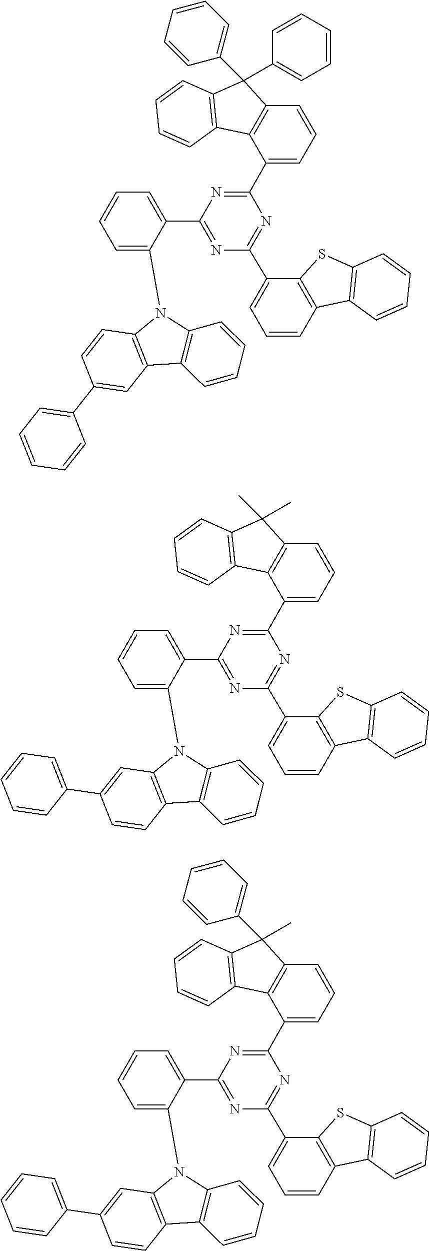

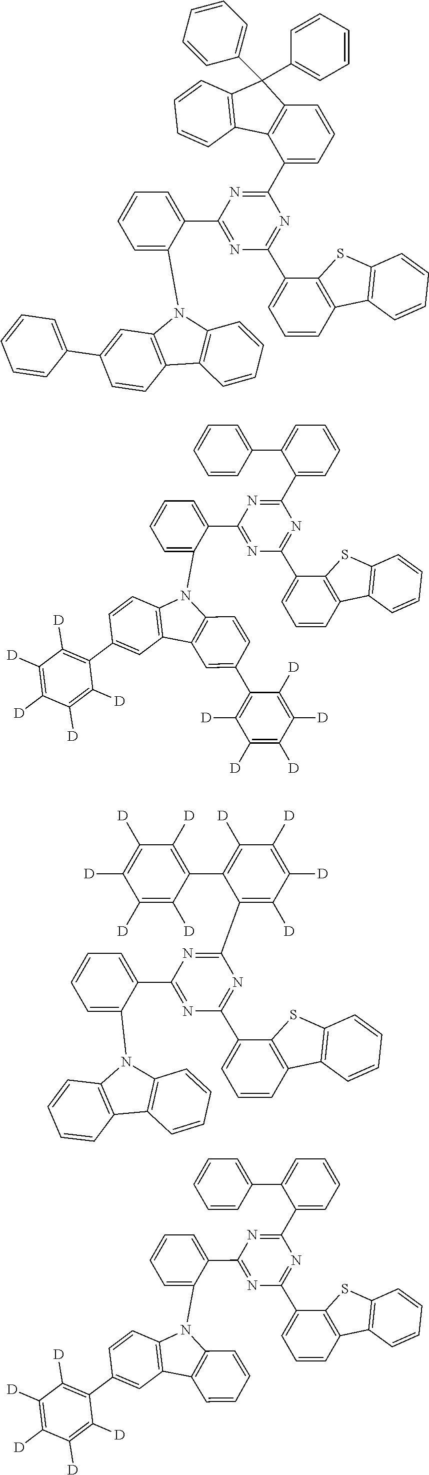

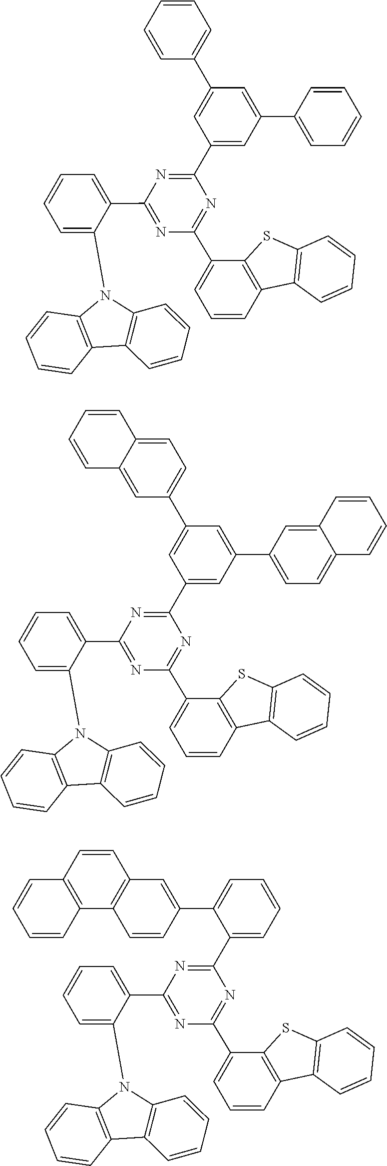

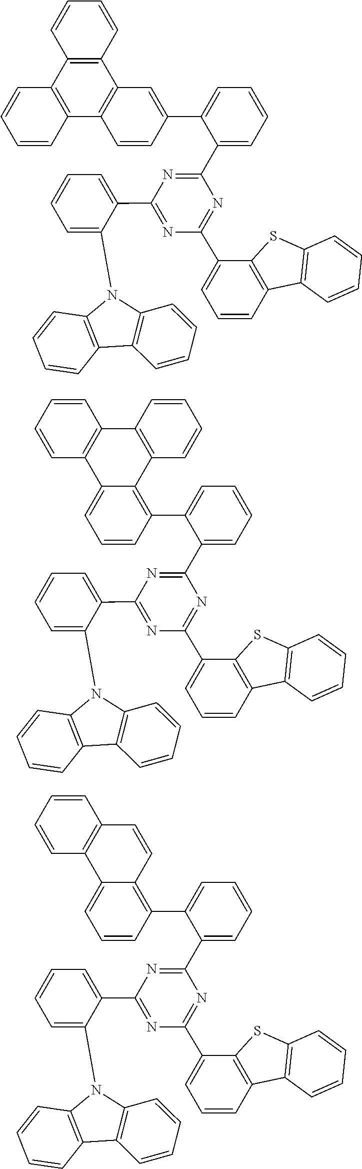

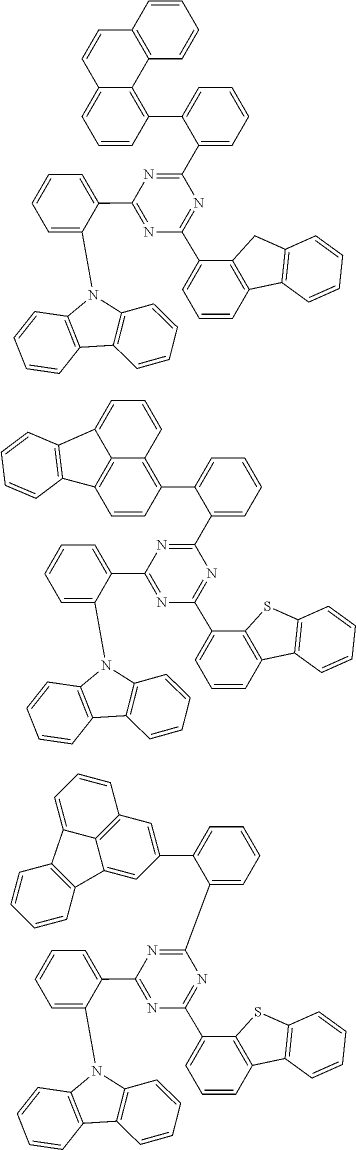

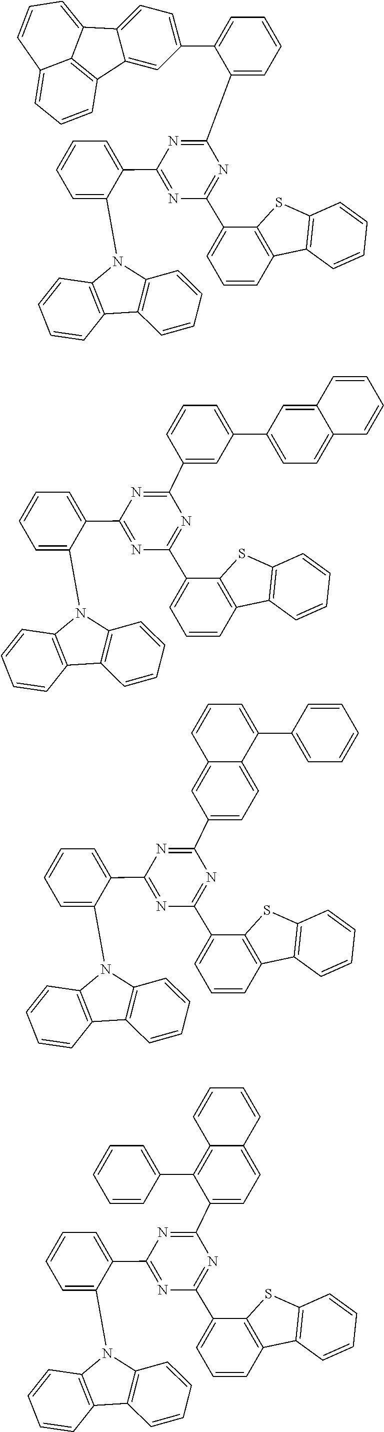 [SMILES: c1ccc(-c2nc(-c3ccccc3-n3c4ccccc4c4ccccc43)nc(-c3cccc4c3sc3ccccc34)n2)c(-c2ccc3c(c2)-c2cccc4cccc-3c24)c1 ; c1cc(-c2ccc3ccccc3c2)cc(-c2nc(-c3ccccc3-n3c4ccccc4c4ccccc43)nc(-c3cccc4c3sc3ccccc34)n2)c1 ; c1ccc(-c2cccc3cc(-c4nc(-c5ccccc5-n5c6ccccc6c6ccccc65)nc(-c5cccc6c5sc5ccccc56)n4)ccc23)cc1 ; c1ccc(-c2c(-c3nc(-c4ccccc4-n4c5ccccc5c5ccccc54)nc(-c4cccc5c4sc4ccccc45)n3)ccc3ccccc23)cc1]

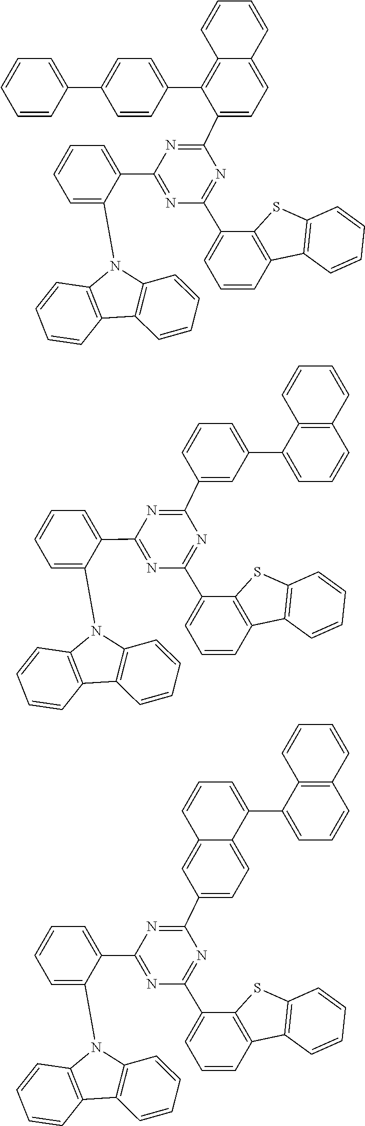 [SMILES: c1ccc(-c2ccc(-c3c(-c4nc(-c5ccccc5-n5c6ccccc6c6ccccc65)nc(-c5cccc6c5sc5ccccc56)n4)ccc4ccccc34)cc2)cc1 ; c1cc(-c2nc(-c3ccccc3-n3c4ccccc4c4ccccc43)nc(-c3cccc4c3sc3ccccc34)n2)cc(-c2cccc3ccccc23)c1 ; c1ccc(-n2c3ccccc3c3ccccc32)c(-c2nc(-c3ccc4c(-c5cccc6ccccc56)cccc4c3)nc(-c3cccc4c3sc3ccccc34)n2)c1]

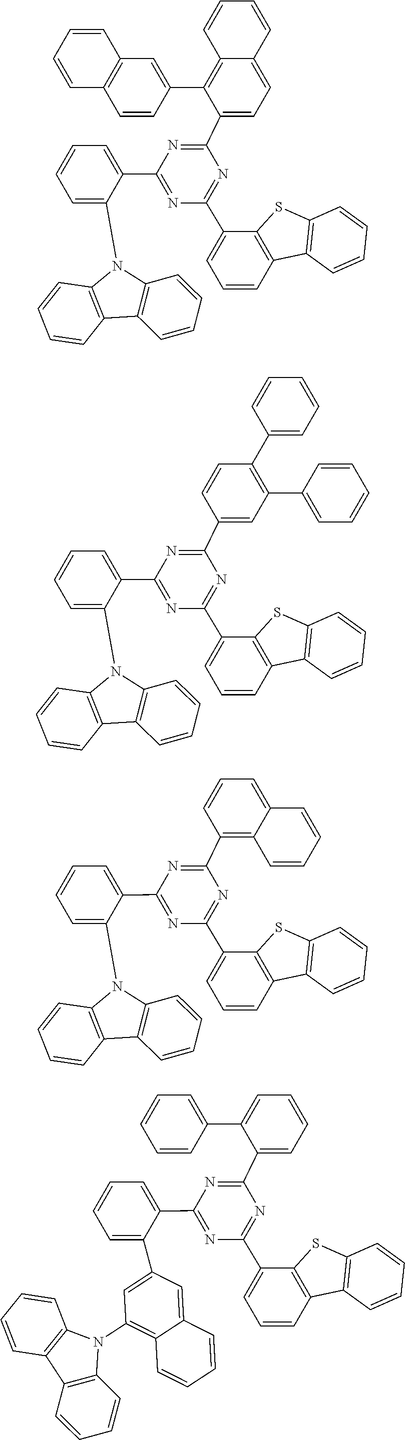

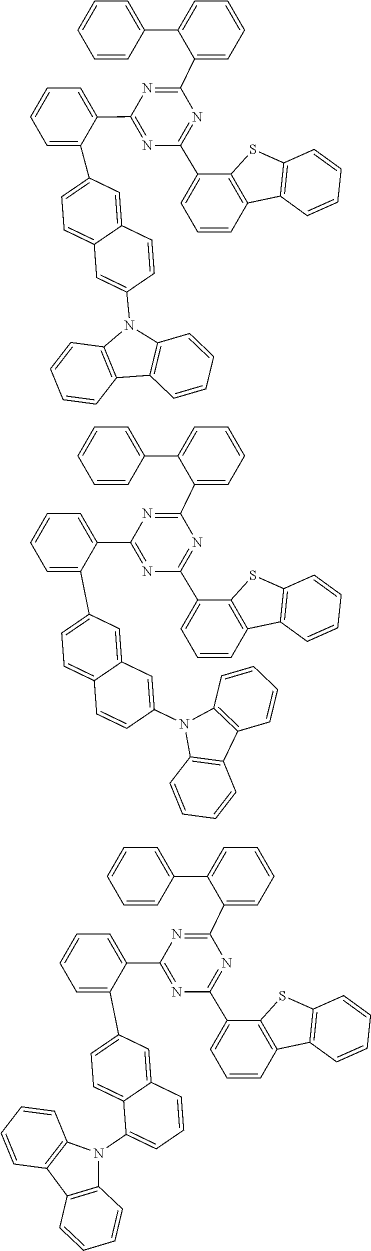

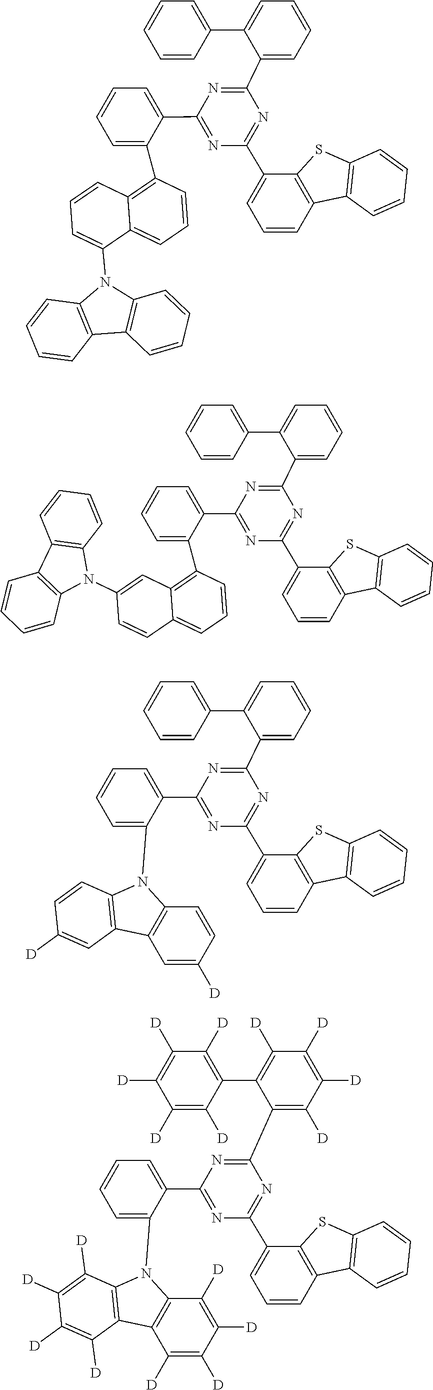

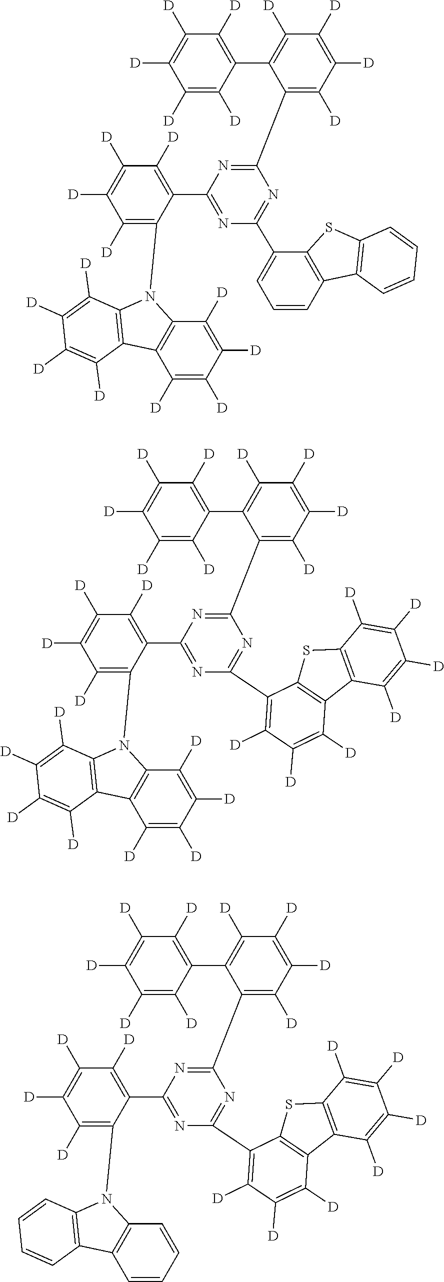

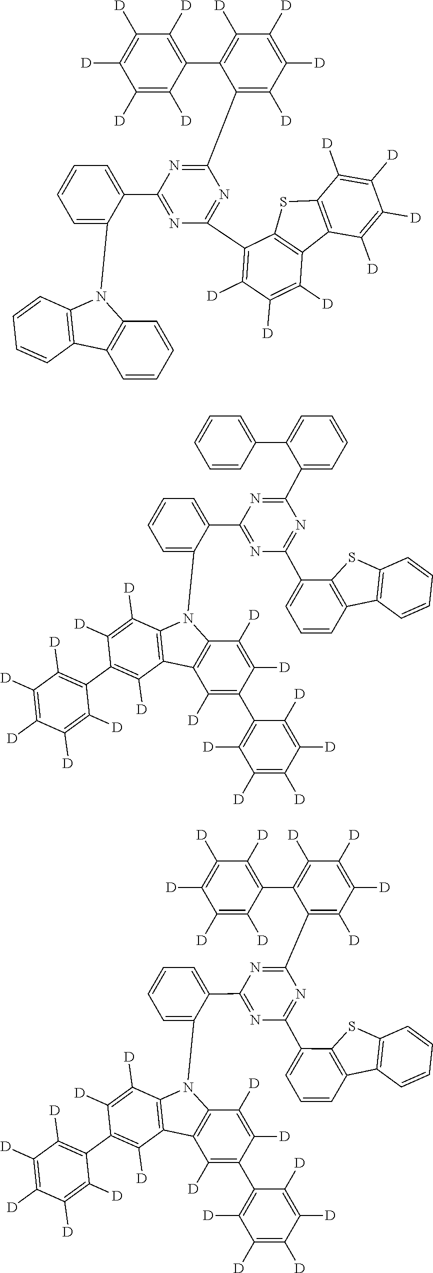

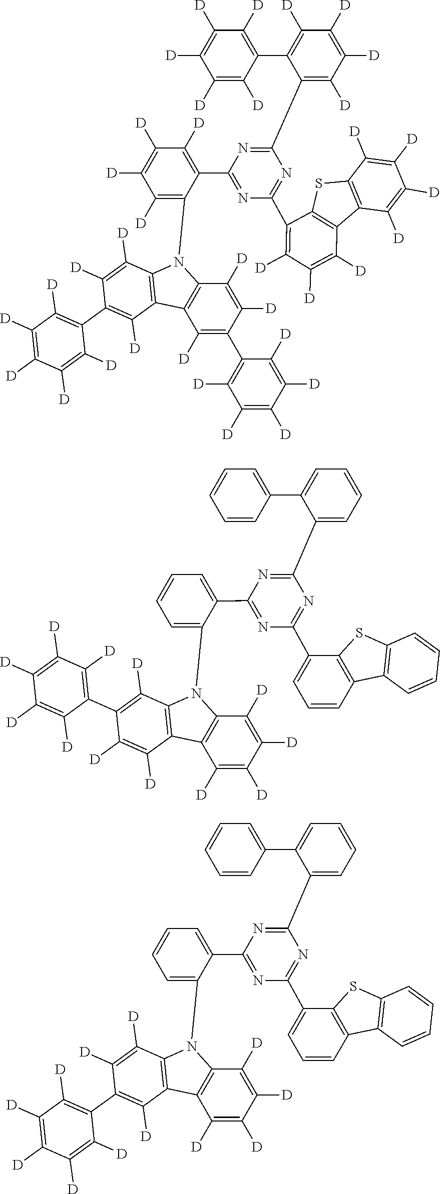

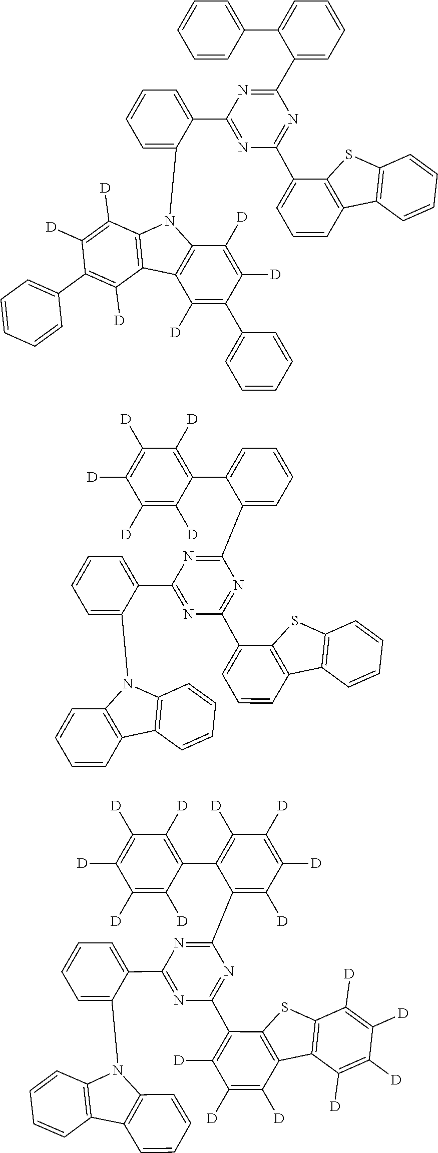

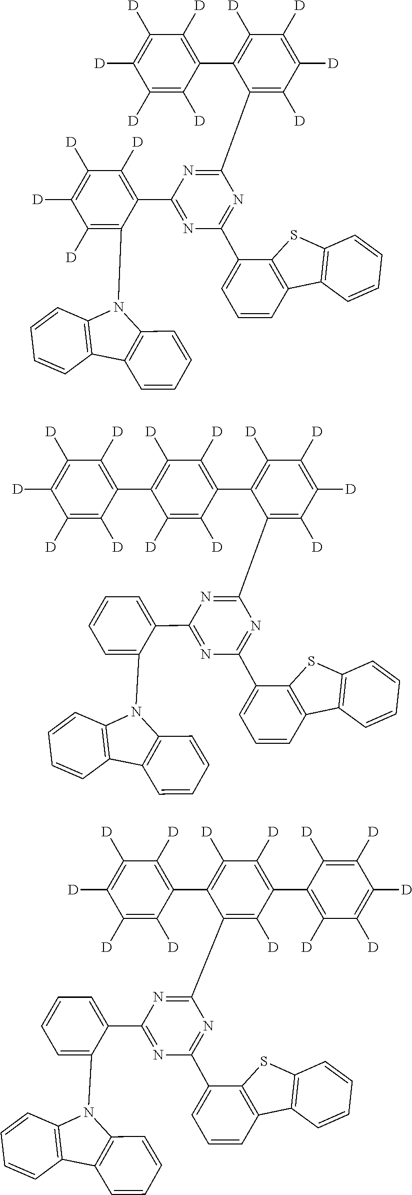

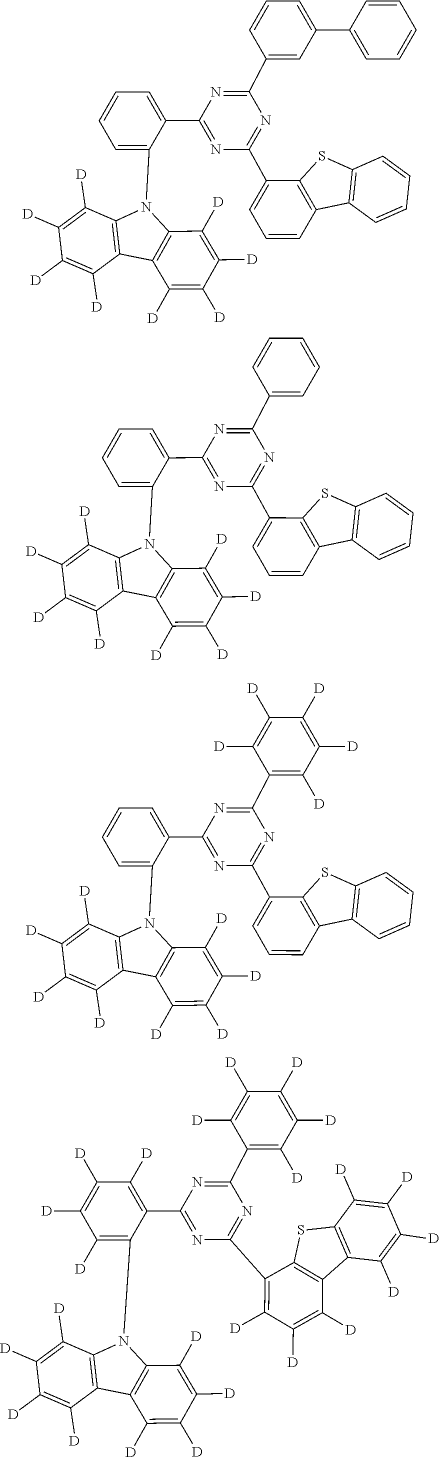

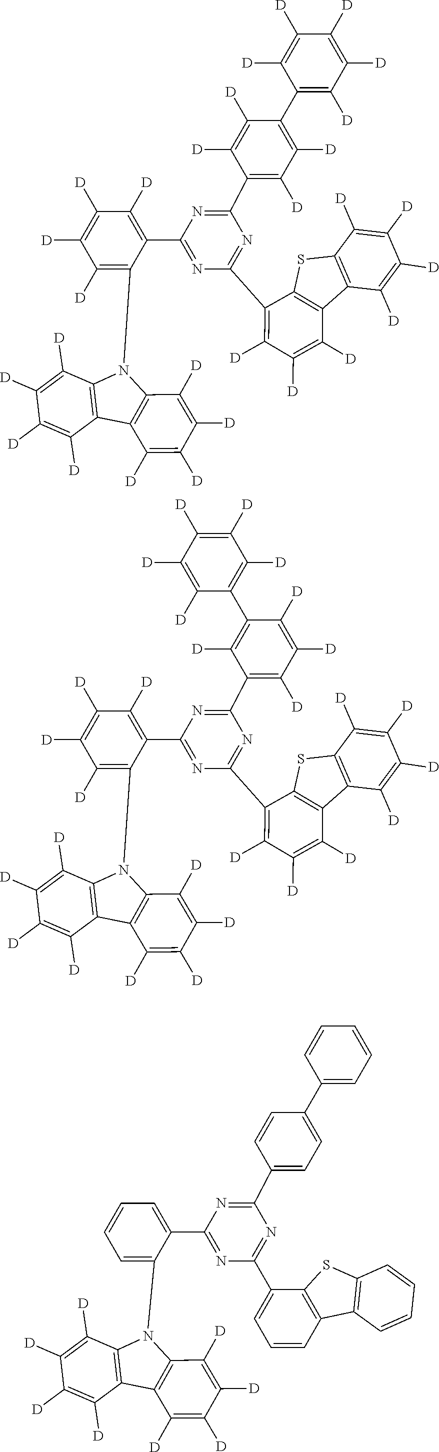

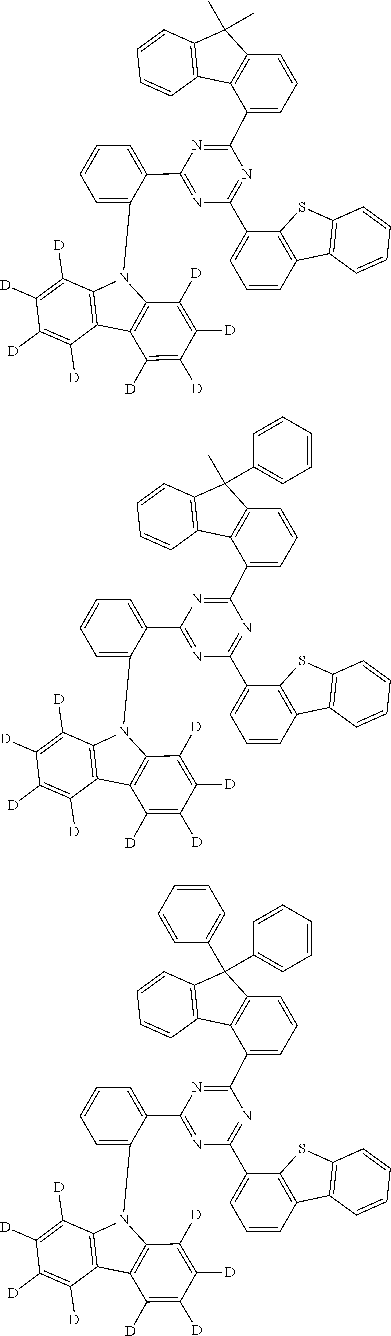

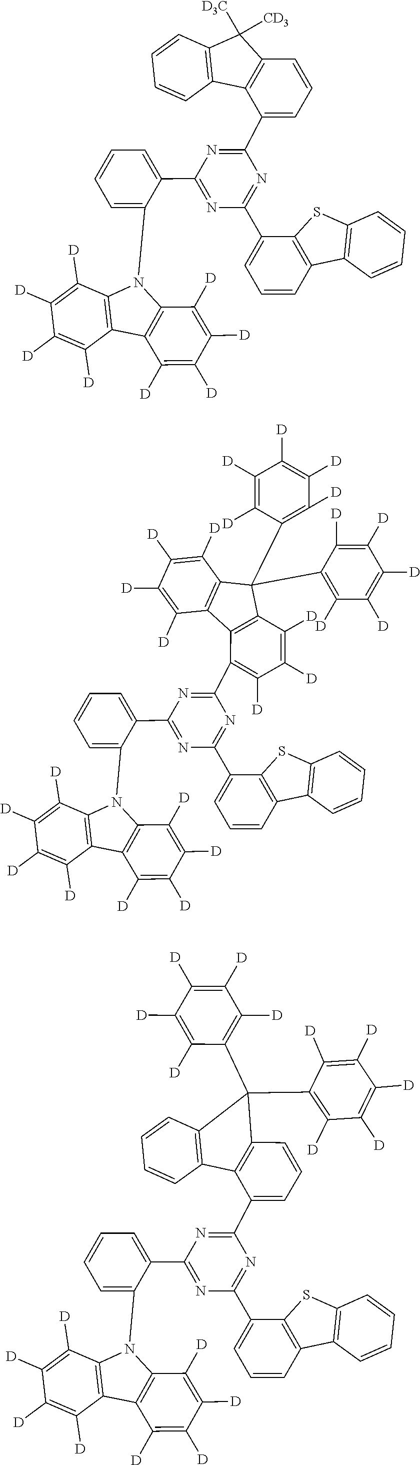

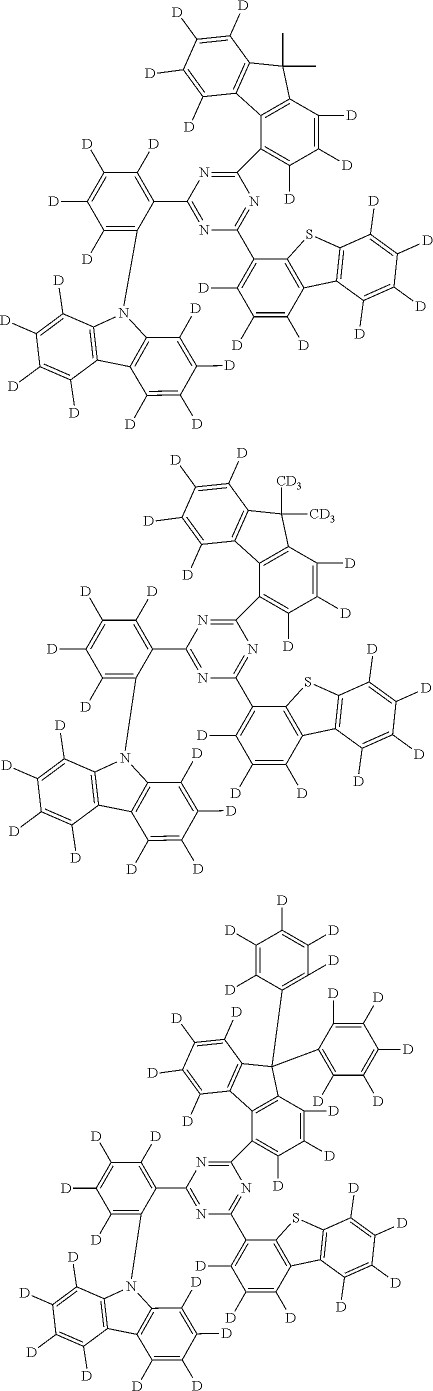

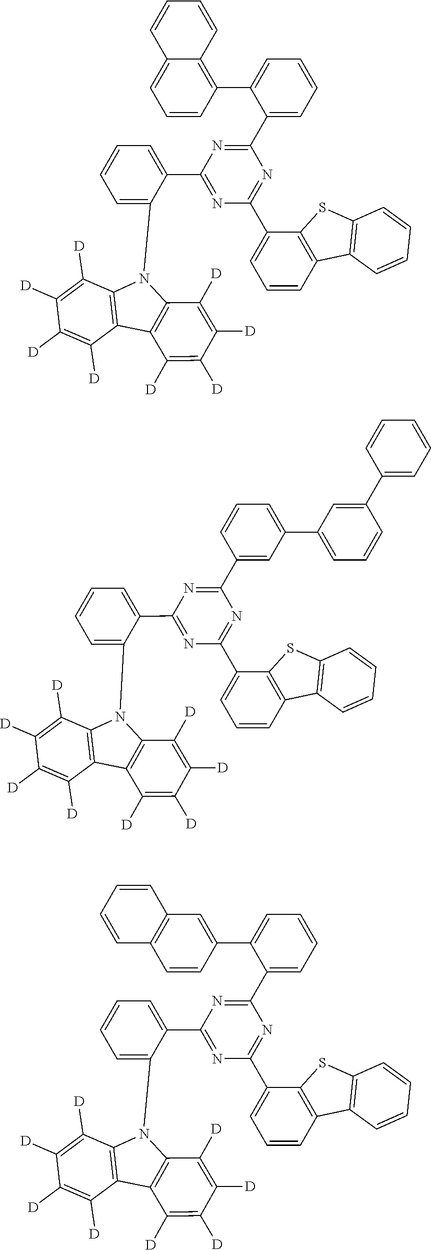 [SMILES: [2H]c1c([2H])c([2H])c2c(c1[2H])c1c([2H])c([2H])c([2H])c([2H])c1n2-c1ccccc1-c1nc(-c2ccccc2-c2cccc3ccccc23)nc(-c2cccc3c2sc2ccccc23)n1 ; [2H]c1c([2H])c([2H])c2c(c1[2H])c1c([2H])c([2H])c([2H])c([2H])c1n2-c1ccccc1-c1nc(-c2cccc(-c3cccc(-c4ccccc4)c3)c2)nc(-c2cccc3c2sc2ccccc23)n1 ; [2H]c1c([2H])c([2H])c2c(c1[2H])c1c([2H])c([2H])c([2H])c([2H])c1n2-c1ccccc1-c1nc(-c2ccccc2-c2ccc3ccccc3c2)nc(-c2cccc3c2sc2ccccc23)n1]

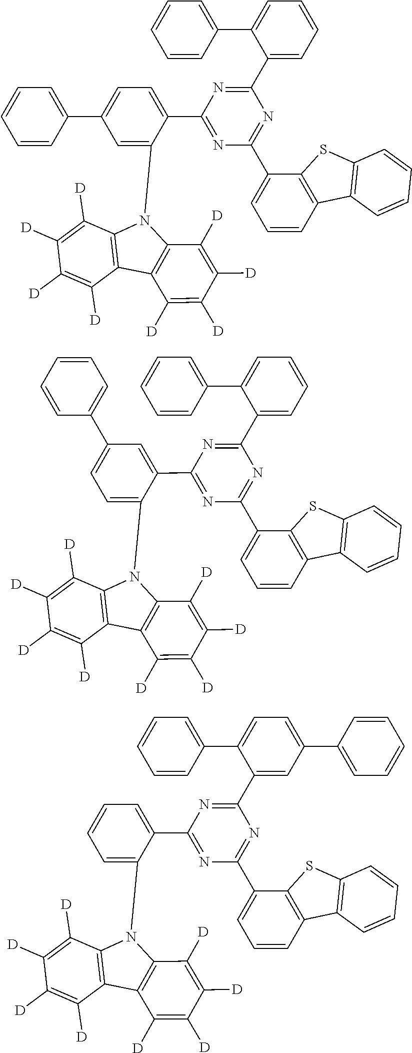

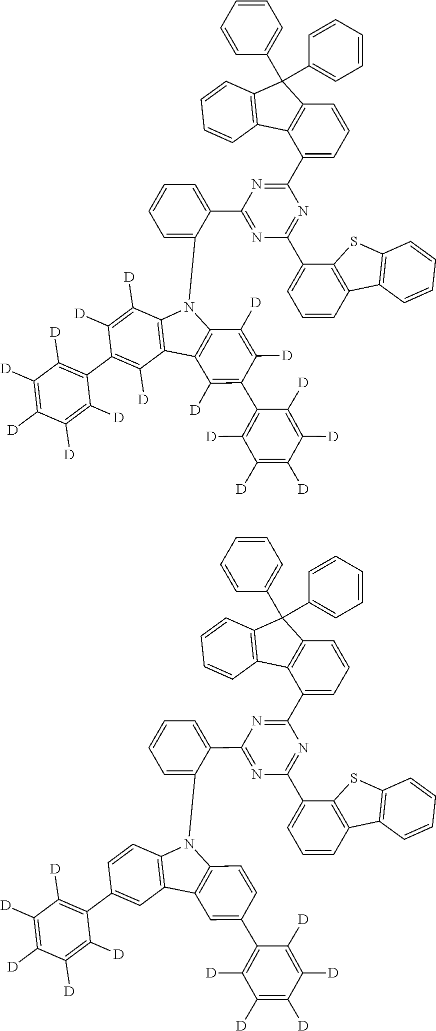 [SMILES: [2H]c1c([2H])c([2H])c(-c2c([2H])c([2H])c3c(c2[2H])c2c([2H])c(-c4c([2H])c([2H])c([2H])c([2H])c4[2H])c([2H])c([2H])c2n3-c2ccccc2-c2nc(-c3cccc4c3-c3ccccc3C4(c3ccccc3)c3ccccc3)nc(-c3cccc4c3sc3ccccc34)n2)c([2H])c1[2H] ; [2H]c1c([2H])c([2H])c(-c2ccc3c(c2)c2cc(-c4c([2H])c([2H])c([2H])c([2H])c4[2H])ccc2n3-c2ccccc2-c2nc(-c3cccc4c3-c3ccccc3C4(c3ccccc3)c3ccccc3)nc(-c3cccc4c3sc3ccccc34)n2)c([2H])c1[2H]]

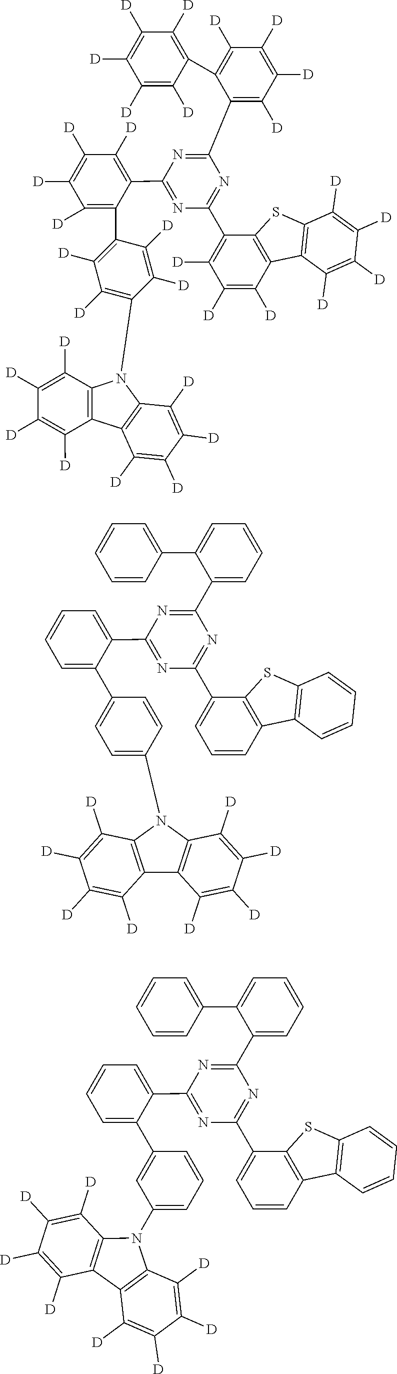

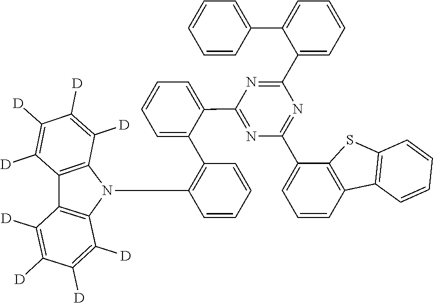 [SMILES: [2H]c1c([2H])c([2H])c2c(c1[2H])c1c([2H])c([2H])c([2H])c([2H])c1n2-c1ccccc1-c1ccccc1-c1nc(-c2ccccc2-c2ccccc2)nc(-c2cccc3c2sc2ccccc23)n1]

[Material for organic electroluminescence device]

The compound according to an aspect of the invention is useful as a material for an organic EL device, and for example, is useful as a material used for an electron-transporting zone of an organic EL device.

### [Organic EL Device]

The organic EL device according to an aspect of the invention will be described.

The organic EL device according to an aspect of the invention has a cathode, an anode, and one or two or more organic layers disposed between the cathode and the anode, and at least one of the organic layers contain the compound according to an aspect of the invention.

The organic EL device according to an aspect of the invention preferably has an anode, an emitting layer, an electron-transporting zone, and a cathode in this order, and the electron-transporting zone contains a compound according to an aspect of the invention.

As typical device configurations of the organic EL device, structures obtained by stacking each of the following structures (1) to (4) and the like on a substrate can be exemplified.

(1) anode/emitting layer/cathode,
(2) anode/hole-transporting zone/emitting layer/cathode
(3) anode/emitting layer/electron-transporting zone/cathode
(4) anode/hole-transporting zone/emitting layer/electron-transporting zone/cathode
“/” indicates that the layers are stacked directly adjacent to each other.

The electron-transporting zone is typically composed of one or more layers selected from an electron-injecting layer and an electron-transporting layer. The hole-transporting zone is typically composed of one or more layers selected from a hole-injecting layer and a hole-transporting layer.

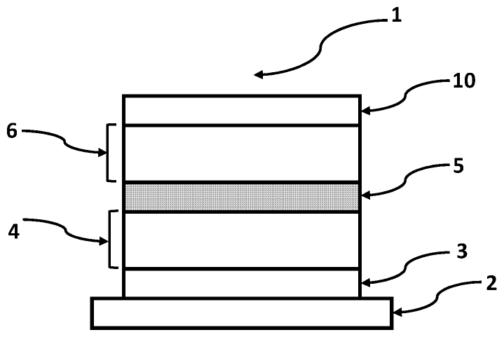

Schematic configuration of the organic EL device of an aspect of the invention will be explained by referring to FIG. 1.

Organic EL device 1 according to an aspect of the invention has a substrate 2, an anode 3, an emitting layer 5, a cathode 10, a hole-transporting zone 4 disposed between the anode 3 and the emitting layer 5, and an electron-transporting zone 6 disposed between the emitting layer 5 and the cathode 10.

Parts which can be used in the organic EL device according to an aspect of the invention, materials for forming respective layers, other than the above-mentioned compounds, and so on, will be described later.

### (Substrate)

A substrate is used as a support of an emitting device. As the substrate, glass, quartz, plastic or the like can be used, for example. Further, a flexible substrate may be used. The “flexible substrate” means a bendable (flexible) substrate, and specific examples thereof include plastic substrates formed of each of polycarbonate and polyvinyl chloride, and the like.

### (Anode)

For the anode formed on the substrate, metals, alloys, electrically conductive compounds, mixtures thereof, and the like, which have a large work function (specifically 4.0 eV or higher) are preferably used. Specific examples thereof include indium oxide-tin oxide (ITO: Indium Tin Oxide), indium oxide-tin oxide containing silicon or silicon oxide, indium oxide-zinc oxide, tungsten oxide, indium oxide containing zinc oxide, graphene, and the like. In addition thereto, specific examples thereof include gold (Au), platinum (Pt), nitrides of a metallic material (for example, titanium nitride), and the like.

### (Hole-Injecting Layer)

The hole-injecting layer is a layer containing a substance having a high hole-injecting property. As such a substance having a high hole-injecting property, molybdenum oxide, titanium oxide, vanadium oxide, rhenium oxide, ruthenium oxide, chromium oxide, zirconium oxide, hafnium oxide, tantalum oxide, silver oxide, tungsten oxide, manganese oxide, aromatic amine compounds, or polymer compounds (oligomers, dendrimers, polymers, etc.) can be given.

### (Hole-Transporting Layer)

The hole-transporting layer is a layer containing a substance having a high hole-transporting property. For the hole-transporting layer, aromatic amine compounds, carbazole derivatives, anthracene derivatives, and the like can be used. Polymer compounds such as poly(N-vinylcarbazole) (abbreviation: PVK) and poly(4-vinyltriphenylamine) (abbreviation: PVTPA) can also be used. However, substances other than the above-described substances may be used as long as the substances have a higher hole-transporting property in comparison with an electron-transporting property. It should be noted that the layer containing the substance having a high hole-transporting property may be not only a single layer, but also a stacking layer composed of two or more layers formed of the above-described substances.

### (Guest (Dopant) Material of Emitting Layer)

The emitting layer is a layer containing a substance having a high emitting property, and various materials can be used for forming it. For example, as the substances having a high emitting property, fluorescent compounds which emit fluorescence or phosphorescent compounds which emit phosphorescence can be used. The fluorescent compound is a compound which can emit from a singlet excited state, and the phosphorescent compound is a compound which can emit from a triplet excited state.

As blue fluorescent emitting materials which can be used for an emitting layer, pyrene derivatives, styrylamine derivatives, chrysene derivatives, fluoranthene derivatives, fluorene derivatives, diamine derivatives, triarylamine derivatives, and the like can be used. As green fluorescent emitting materials which can be used for an emitting layer, aromatic amine derivatives and the like can be used. As red fluorescent emitting materials which can be used for an emitting layer, tetracene derivatives, diamine derivatives and the like can be used.

As blue phosphorescent emitting materials which can be used for an emitting layer, metal complexes such as iridium complexes, osmium complexes, platinum complexes and the like are used. As green phosphorescent emitting materials which can be used for an emitting layer, iridium complexes and the like are used. As red phosphorescent emitting materials which can be used for an emitting layer, metal complexes such as iridium complexes, platinum complexes, terbium complexes, europium complexes and the like are used.

### (Host Material for Emitting Layer)

The emitting layer may have a constitution in which the above-mentioned substance having a high emitting property (guest material) is dispersed in another substance (host material). As substances for dispersing the substance having a high emitting property, a variety of substances can be used, and it is preferable to use a substance having a higher lowest unoccupied orbital level (LUMO level) and a lower highest occupied orbital level (HOMO level) than the substance having a high emitting property.

As substances (host materials) for dispersing the substance having a high emitting property, 1) metal complexes such as aluminum complexes, beryllium complexes, and zinc complexes, 2) heterocyclic compounds such as oxadiazole derivatives, benzimidazole derivatives and phenanthroline derivatives, 3) fused aromatic compounds such as carbazole derivatives, anthracene derivatives, phenanthrene derivatives, pyrene derivatives and chrysene derivatives, and 4) aromatic amine compounds such as triarylamine derivatives and fused polycyclic aromatic amine derivatives are used.

### (Electron-Transporting Layer)

The electron-transporting layer is a layer that contains a substance having a high electron-transporting property. For the electron-transporting layer, 1) metal complexes such as aluminum complexes, beryllium complexes, zinc complexes, and the like; 2) heteroaromatic complexes such as imidazole derivatives, benzimidazole derivatives, azine derivatives, carbazole derivatives, phenanthroline derivatives, and the like; and 3) polymer compounds can be used.

In one embodiment, the electron-transporting layer may or may not contain other substances in addition to the compound represented by the formula (1) described above.

In one embodiment, in addition to the compound represented by the formula (1), the electron-transporting layer contains one or more compounds selected from the group consisting of a compounds containing an alkali metal and compounds containing a metal belonging to Group 13 in the Periodic Table of the Elements. Examples of such compounds include lithium fluoride, lithium oxide, 8-hydroxyquinolinolato-lithium (Liq), cesium fluoride, tris(8-quinolinolato)aluminum (Alq3), tris(4-methyl-8-quinolinolato)aluminum (Almq3), bis(2-methyl-8-quinolinolato)(4-phenylphenolato)aluminum (BAlq), and the like.

The content ratio (mass ratio) of the compound represented by the formula (1) to that of the compound containing an alkali metal and the compound containing a metal belonging to Group 13 in the Periodic Table of the Elements is not particularly limited, and is, for example, 10:90 to 90:10.

In the organic EL device according to an aspect of the invention, the electron-transporting zone has a first layer (also referred to as a “first electron-transporting layer” or a “hole barrier layer”) and a second layer (also referred to as a “second electron-transporting layer”) in this order from the emitting layer side, and the second layer contains the compound represented by the formula (1). As the first layer, the above-described configuration of the electron-transporting layer can be applied.

### (Electron-Injecting Layer)

The electron-injecting layer is a layer which contains a substance having a high electron-injecting property. For the electron-injecting layer, lithium (Li), ytterbium (Yb), lithium fluoride (LiF), cesium fluoride (CsF), calcium fluoride (CaF₂), metal complex compounds such as 8-hydroxyquinolinolato-lithium (Liq), alkali metals, alkaline earth metals and compounds of the alkali metals and the alkaline earth metals such as lithium oxide (LiOₓ) can be used.

### (Cathode)

For the cathode, metals, alloys, electrically conductive compounds, mixtures thereof, and the like having a small work function (specifically, 3.8 eV or lower) are preferably used. Specific examples of such cathode materials include elements belonging to Group 1 or Group 2 of the Periodic Table of the Elements, i.e., alkali metals such as lithium (Li) and cesium (Cs), alkaline earth metals such as magnesium (Mg), calcium (Ca) and strontium (Sr), and alloys containing these metals (e.g., MgAg and AlLi); rare earth metals such as europium (Eu) and ytterbium (Yb), and alloys containing these metals.

The cathode is usually formed by a vacuum vapor deposition or a sputtering method. Further, in the case of using a silver paste or the like, a coating method, an inkjet method, or the like can be employed.

In the case where the electron-injecting layer is provided, a cathode can be formed of a substance selected from various electrically conductive materials such as aluminum, silver, ITO, graphene, indium oxide-tin oxide containing silicon or silicon oxide, regardless of the work function value.

In the organic EL device according to an aspect of the invention, the thickness of each layer is not particularly limited, but is generally preferable that the thickness be in the range of several nm to 1 μm in order to suppress defects such as pinholes, to make applied voltages to be low, and to increase luminous efficiency.

In the organic EL device according to an aspect of the invention, the methods for forming the respective layers are not particularly limited. Conventionally-known methods for forming each layer such as a vacuum deposition process, a spin coating process and the like can be used. Each layer such as the emitting layer can be formed by a known method such as a vacuum deposition process, a molecular beam deposition process (MBE process), or an application process such as a dipping process, a spin coating process, a casting process, a bar coating process, or a roll coating process, using a solution prepared by dissolving the material in a solvent.

### [Electronic Apparatus]

The electronic apparatus according to an aspect of the invention is characterized in that the organic EL device according to an aspect of the invention is equipped with.

Specific examples of the electronic apparatuses include display components such as an organic EL panel module, and the like; display devices for a television, a cellular phone, a personal computer, and the like; and emitting devices such as a light, a vehicular lamp, and the like.

### EXAMPLES

### <Compound>s

The compounds represented by the formula (1) used for fabrication of the organic EL devices of Examples 1 to 12 are shown below

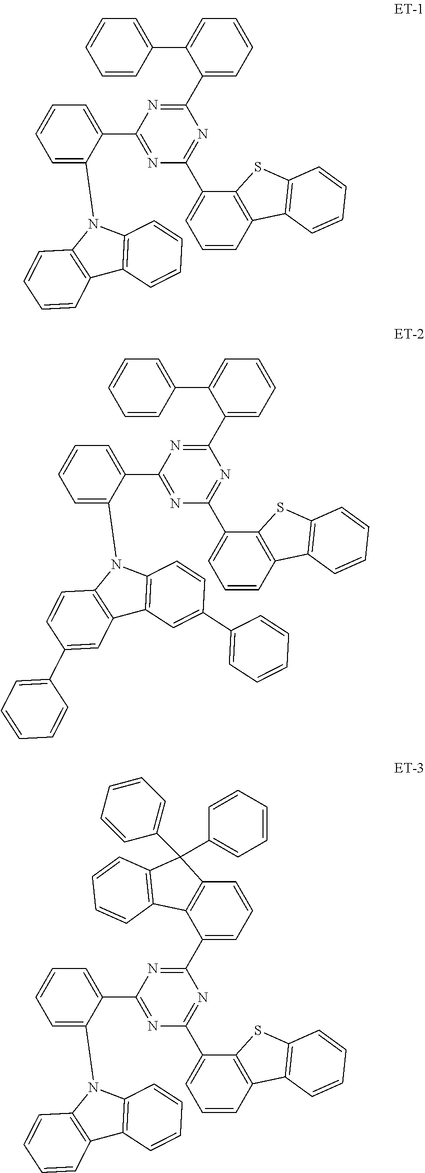 [SMILES: c1ccc(-c2ccccc2-c2nc(-c3ccccc3-n3c4ccccc4c4ccccc43)nc(-c3cccc4c3sc3ccccc34)n2)cc1 ; c1ccc(-c2ccc3c(c2)c2cc(-c4ccccc4)ccc2n3-c2ccccc2-c2nc(-c3ccccc3-c3ccccc3)nc(-c3cccc4c3sc3ccccc34)n2)cc1 ; c1ccc(C2(c3ccccc3)c3ccccc3-c3c(-c4nc(-c5ccccc5-n5c6ccccc6c6ccccc65)nc(-c5cccc6c5sc5ccccc56)n4)cccc32)cc1]

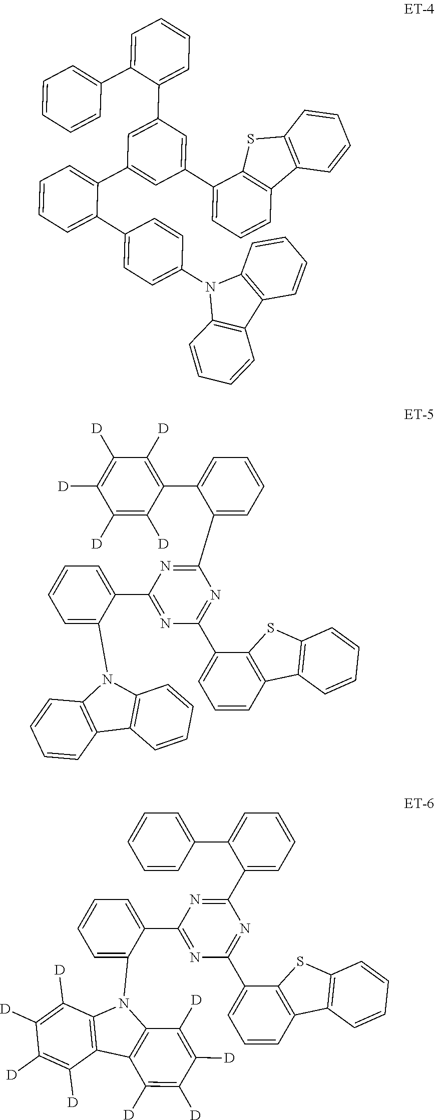 [SMILES: c1ccc(-c2ccccc2-c2cc(-c3ccccc3-c3ccc(-n4c5ccccc5c5ccccc54)cc3)cc(-c3cccc4c3sc3ccccc34)c2)cc1 ; [2H]c1c([2H])c([2H])c(-c2ccccc2-c2nc(-c3ccccc3-n3c4ccccc4c4ccccc43)nc(-c3cccc4c3sc3ccccc34)n2)c([2H])c1[2H] ; [2H]c1c([2H])c([2H])c2c(c1[2H])c1c([2H])c([2H])c([2H])c([2H])c1n2-c1ccccc1-c1nc(-c2ccccc2-c2ccccc2)nc(-c2cccc3c2sc2ccccc23)n1]

The structure of the compound used for fabrication of the organic EL devices of Comparative Examples 1 to 2 is shown below

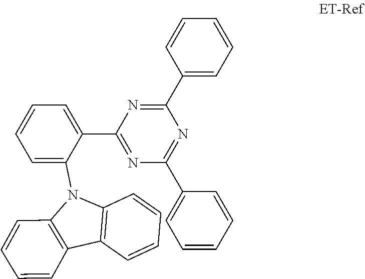 [SMILES: c1ccc(-c2nc(-c3ccccc3)nc(-c3ccccc3-n3c4ccccc4c4ccccc43)n2)cc1]

The structures of the other compounds used for fabrication of the organic EL devices of Examples 1 to 12 and Comparative Examples 1 to 2 are shown below.

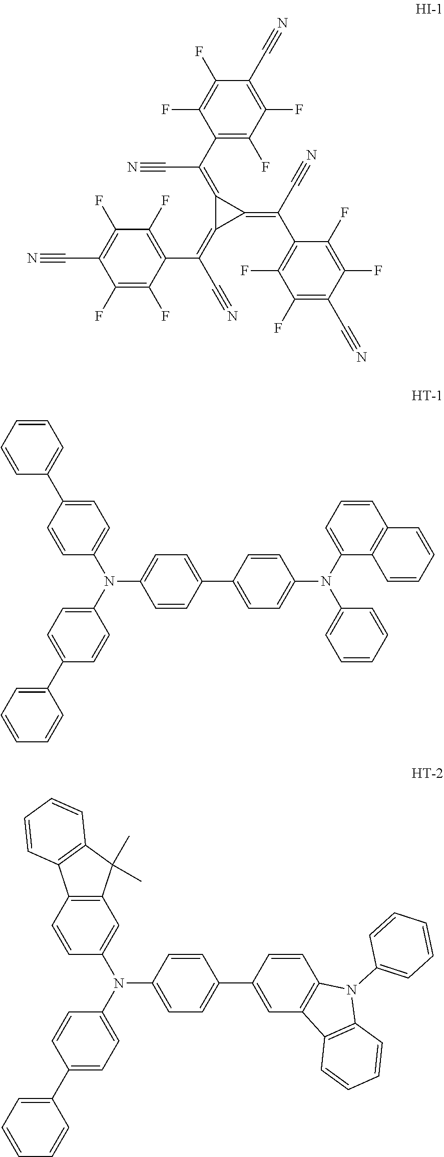 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; c1ccc(-c2ccc(N(c3ccc(-c4ccccc4)cc3)c3ccc(-c4ccc(N(c5ccccc5)c5cccc6ccccc56)cc4)cc3)cc2)cc1 ; CC1(C)c2ccccc2-c2ccc(N(c3ccc(-c4ccccc4)cc3)c3ccc(-c4ccc5c(c4)c4ccccc4n5-c4ccccc4)cc3)cc21]

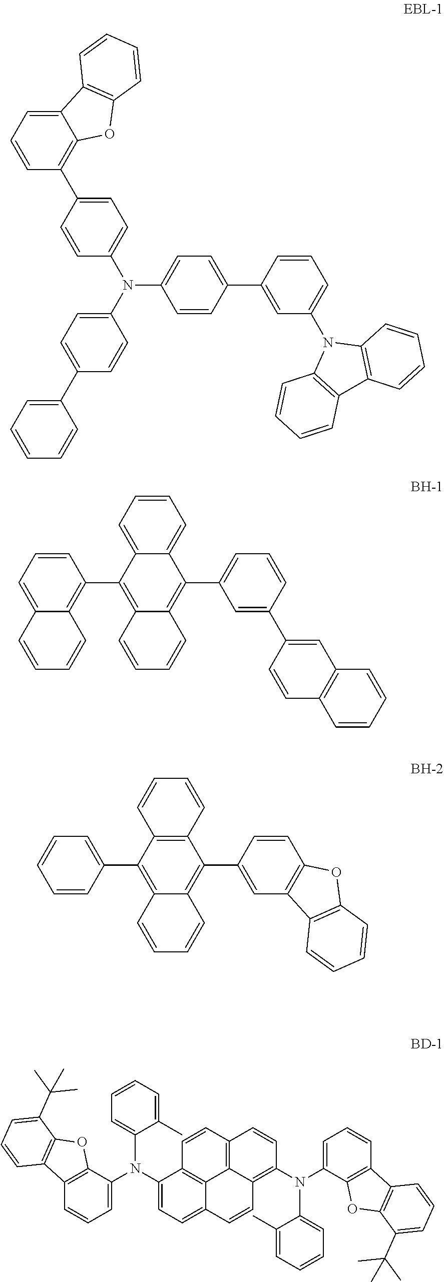

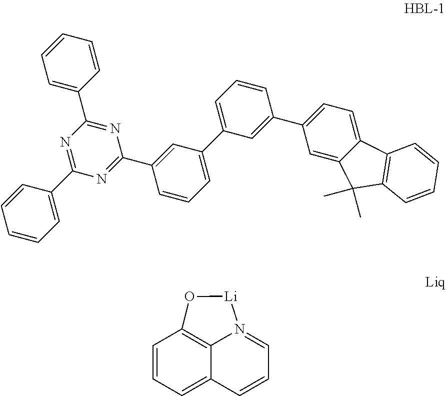

### Example 1

### <Fabrication of Organic EL Device>

An organic EL device was fabricated as follows.

A 25 mm×75 mm×1.1 mm-thick glass substrate with an ITO transparent electrode (anode) (manufactured by GEOMATEC Co., Ltd.) was subjected to ultrasonic cleaning in isopropyl alcohol for 5 minutes, and then subjected to UV-ozone cleaning for 30 minutes. The thickness of the ITO film was 130 nm.

The glass substrate with the transparent electrode after being cleaned was mounted onto a substrate holder in a vacuum vapor deposition apparatus. First, a compound HT-1 and a compound HI-1 were co-deposited so as to be 3% by mass in a proportion of the compound HI-1 on a surface on the side on which the transparent electrode was formed so as to cover the transparent electrode to form a first hole-transporting layer having a thickness of 10 nm.

On the first hole-transporting layer, the compound HT-1 was deposited to form a second hole-transporting layer having a thickness of 80 nm.

On the second hole-transporting layer, a compound EBL-1 was deposited to form a third hole-transporting layer having a thickness of 5 nm.

A compound BH-1 (a host material) and a compound BD-1 (a dopant material) were co-deposited on the third hole-transporting layer so as to be 4% by mass in a proportion of the compound BD-1 to form an emitting layer having a thickness of 25 nm.

A compound HBL-1 was deposited on the emitting layer to form a first electron-transporting layer having a thickness of 5 nm.

A compound ET-1 and 8-hydroxyquinolinolato-lithium (Liq) were co-deposited on the first electron-transporting layer so as to be 50% by mass in a proportion of Liq to form a second electron-transporting layer having a thickness of 20 nm.

On the second electron-transporting layer, metal Yb was deposited to form an electron-injecting layer having a thickness of 1 nm.

Metal Al was deposited on the electron-injecting layer to form a cathode having a thickness of 50 nm.

The device configuration of the organic EL device of Example 1 is schematically shown as follows. ITO(130)/HT-1:HI-1(10:3%)/HT-1(80)/EBL-1(5)/BH-1:BD-1(25:4%)/HBL-1(5)/ET-1:Liq(20:50%)/Yb(1)/Al(50)

Numerical values in parentheses indicate a film thickness (unit: nm). In parentheses, the numerical values in percentage indicate the proportion (% by mass) of the latter compound in the layer.

### <Evaluation of Organic EL Device>

For the resulting organic EL device, voltage was applied at room temperature to the organic EL device so as to be 50 mA/cm² in current density, and the time until the luminance reduces to 95% of the initial luminance (LT95 (unit: h)) was measured.

### Examples 2 to 6

Organic EL devices were fabricated and evaluated in the same manner as in Example 1, except that the compounds described in Table 1 were used respectively in place of the compound ET-1. The results are shown in Table 1.

### Comparative Example 1

An organic EL device was fabricated and evaluated in the same manner as in Example 1, except that the compound described in Table 1 was used in place of the compound ET-1. The results are shown in Table 1.

TABLE 1

| | Second electron- | | |
|---|---|---|---|
| | transporting layer | | LT95(hr) |
| Example 1 | ET-1 | Liq | 92 |
| Example 2 | ET-2 | Liq | 86 |
| Example 3 | ET-3 | Liq | 109 |
| Example 4 | ET-4 | Liq | 88 |
| Example 5 | ET-5 | Liq | 94 |
| Example 6 | ET-6 | Liq | 96 |
| Comp. Ex. 1 | ET-Ref | Liq | 50 |

### Example 7

### <Fabrication of Organic EL Device>

An organic EL device was fabricated as follows.

A 25 mm×75 mm×1.1 mm-thick glass substrate with an ITO transparent electrode (anode) (manufactured by GEOMATEC Co., Ltd.) was subjected to ultrasonic cleaning in isopropyl alcohol for 5 minutes, and then subjected to UV-ozone cleaning for 30 minutes. The thickness of the ITO film was 130 nm.

The glass substrate with the transparent electrode after being cleaned was mounted onto a substrate holder in a vacuum vapor deposition apparatus. First, a compound HT-2 and a compound HI-1 were co-deposited so as to be 3% by mass in a proportion of the compound HI-1 on a surface on the side on which the transparent electrode was formed so as to cover the transparent electrode to form a first hole-transporting layer having a thickness of 10 nm.

On the first hole-transporting layer, a compound HT-2 was deposited to form a second hole-transporting layer having a thickness of 80 nm.

On the second hole-transporting layer, a compound EBL-1 was deposited to form a third hole-transporting layer having a thickness of 5 nm.

A compound BH-2 (a host material) and a compound BD-1 (a dopant material) were co-deposited on the third hole-transporting layer so as to be 4% by mass in a proportion of the compound BD-1 to form an emitting layer having a thickness of 25 nm.

A compound HBL-1 was deposited on the emitting layer to form a first electron-transporting layer having a thickness of 5 nm.

A compound ET-1 and Liq were co-deposited on the first electron-transporting layer so as to be 50% by mass in a proportion of Liq to form a second electron-transporting layer having a thickness of 20 nm.

On the second electron-transporting layer, metal Yb was deposited to form an electron-injecting layer having a thickness of 1 nm.

Metal Al was deposited on the electron-injecting layer to form a cathode having a thickness of 50 nm.

The device configuration of the organic EL device of Example 7 is schematically shown as follows. ITO(130)/HT-2:HI-1(10:3%)/HT-2(80)/EBL-1(5)/BH-2:BD-1(25:4%)/HBL-1(5)/ET-1:Liq(20:50%)/Yb(1)/Al(50)

Numerical values in parentheses indicate a film thickness (unit: nm). In parentheses, the numerical values in percentage indicate the proportion (% by mass) of the latter compound in the layer.

### <Evaluation of Organic EL Device>

The obtained organic EL device was evaluated in the same manner as in Example 1.

### Examples 8 to 12

Organic EL devices were fabricated and evaluated in the same manner as in Example 7, except that the compounds described in Table 2 was used respectively in place of the compound ET-1. The results are shown in Table 2.

### Comparative Example 2

An organic EL device was fabricated and evaluated in the same manner as in Example 7, except that the compound described in Table 2 was used in place of the compound ET-1. The results are shown in Table 2.

TABLE 2

| | Second electron- | | |
|---|---|---|---|
| | transporting layer | | LT95(hr) |
| Example 7 | ET-1 | Liq | 63 |
| Example 8 | ET-2 | Liq | 58 |
| Example 9 | ET-3 | Liq | 70 |
| Example 10 | ET-4 | Liq | 60 |
| Example 11 | ET-5 | Liq | 65 |
| Example 12 | ET-6 | Liq | 68 |
| Comp. Ex. 2 | ET-Ref | Liq | 31 |

### <Synthesis of Compounds>

### (Synthesis Example 1) Synthesis of ET-1

The compound ET-1 was synthesized in accordance with the synthetic route described below.

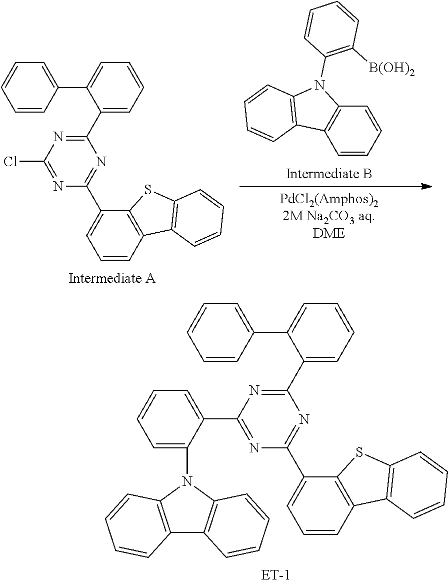 [SMILES: Clc1nc(-c2ccccc2-c2ccccc2)nc(-c2cccc3c2sc2ccccc23)n1 ; OB(O)c1ccccc1-n1c2ccccc2c2ccccc21 ; c1ccc(-c2ccccc2-c2nc(-c3ccccc3-n3c4ccccc4c4ccccc43)nc(-c3cccc4c3sc3ccccc34)n2)cc1]

Intermediate A (3.0 g) synthesized in accordance with the method described in WD 2020/116615 and Intermediate B (2.9 g) synthesized in accordance with the method described in WD 2019/189033 were added to 1,2-dimethoxyethane (DME) (130 mL), and the solution was passed through argon gas for 5 minutes. To this solution, PdCl₂(Amphos)₂ (0.19 g) and an aqueous solution of sodium carbonate (2 M, 8.3 mL) were added, and the mixture was heated at 80° C. for 6 hours while stirring under an argon atmosphere. The solvent of the reaction solution was distilled off, and the obtained solid was purified by silica gel column chromatography (developing solvent; hexane/toluene) to obtain ET-1 as a white solid (3.6 g, yield: 82%).

The result of mass spectral analysis was m/e=656 for a molecular weight of 656.81, thereby the product was identified as the target compound.

### (Synthesis Example 2) Synthesis of ET-2

### (2-1) Synthesis of Intermediate C

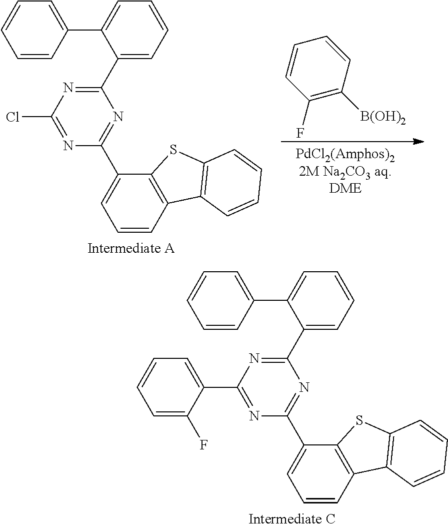 [SMILES: Clc1nc(-c2ccccc2-c2ccccc2)nc(-c2cccc3c2sc2ccccc23)n1 ; OB(O)c1ccccc1F ; Fc1ccccc1-c1nc(-c2ccccc2-c2ccccc2)nc(-c2cccc3c2sc2ccccc23)n1]

Intermediate A (5.0 g) and 2-fluorophenylboronic acid (1.7 g) were used under the same conditions as described in Synthesis Example 1, to obtain an Intermediate C as a white solid (5.2 g, yield: 89%).

### (2-2) Synthesis of ET-2

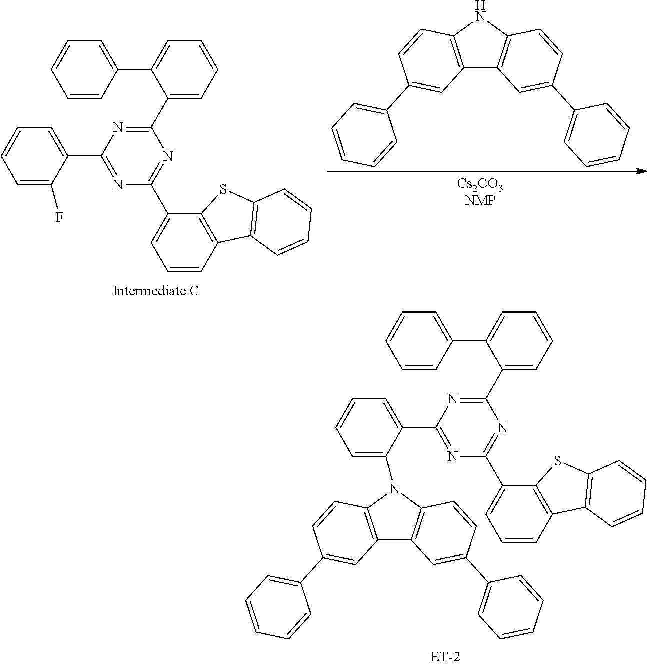 [SMILES: Fc1ccccc1-c1nc(-c2ccccc2-c2ccccc2)nc(-c2cccc3c2sc2ccccc23)n1 ; c1ccc(-c2ccc3[nH]c4ccc(-c5ccccc5)cc4c3c2)cc1 ; c1ccc(-c2ccc3c(c2)c2cc(-c4ccccc4)ccc2n3-c2ccccc2-c2nc(-c3ccccc3-c3ccccc3)nc(-c3cccc4c3sc3ccccc34)n2)cc1]

Intermediate C (5.2 g), 3,6-diphenylcarbazole (3.9 g), and cesium carbonate (10.0 g) were added to N-methylpyrrolidone (NMP) (60 mL), and the mixture was heated at 150° C. for 24 hours with stirring under an argon atmosphere. The solid precipitated by addition of methanol and water was dissolved in toluene and passed through silica gel. The discharged liquid was concentrated and the resulting solid was subjected to suspension wash with ethanol, and further purified by recrystallization from toluene repeatedly to obtain ET-2 as a white solid (6.5 g, yield: 78%).

The result of mass spectral analysis was m/e=809 for a molecular weight of 809.00, thereby the product was identified as the target compound.

### (Synthesis Example 3) Synthesis of ET-3

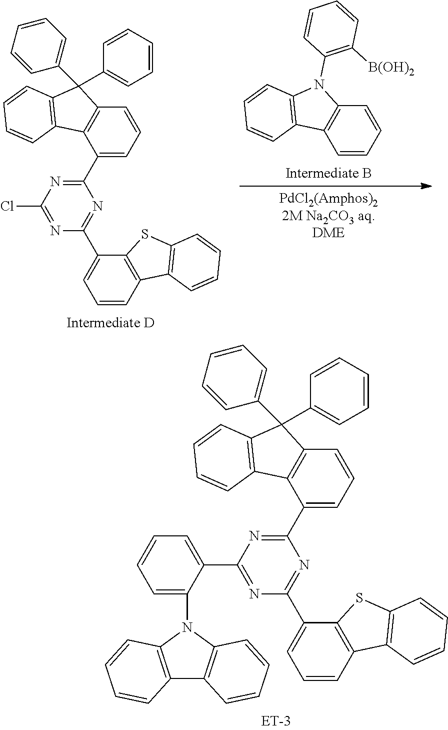 [SMILES: Clc1nc(-c2cccc3c2-c2ccccc2C3(c2ccccc2)c2ccccc2)nc(-c2cccc3c2sc2ccccc23)n1 ; OB(O)c1ccccc1-n1c2ccccc2c2ccccc21 ; c1ccc(C2(c3ccccc3)c3ccccc3-c3c(-c4nc(-c5ccccc5-n5c6ccccc6c6ccccc65)nc(-c5cccc6c5sc5ccccc56)n4)cccc32)cc1]

Intermediate D (3.0 g) synthesized in accordance with the method described in WD 2021/033724 and Intermediate B (2.1 g) were used to obtain ET-3 as a white solid (3.4 g, yield: 85%) under the same conditions as described in (1-2) of Synthesis Example 1.

The result of mass spectral analysis was m/e=821 for a molecular weight of 821.01, thereby the product was identified as the target compound.

### (Synthesis Example 4) Synthesis of ET-4

### (4-1) Synthesis of Intermediate E

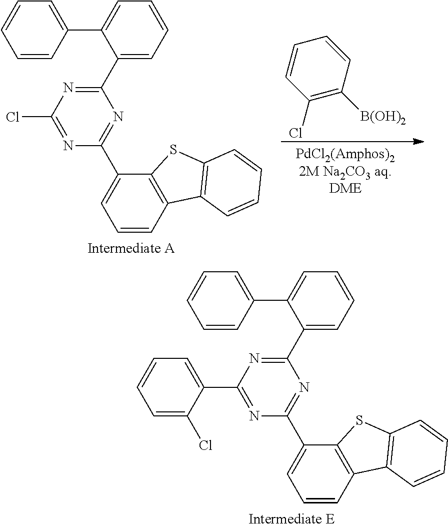 [SMILES: Clc1nc(-c2ccccc2-c2ccccc2)nc(-c2cccc3c2sc2ccccc23)n1 ; OB(O)c1ccccc1Cl ; Clc1ccccc1-c1nc(-c2ccccc2-c2ccccc2)nc(-c2cccc3c2sc2ccccc23)n1]

Intermediate A (5.0 g) and 2-chlorophenylboronic acid (1.9 g) were used to obtain Intermediate E as a white solid (5.0 g, yield: 86%) under the same conditions as described in Synthesis Example 1.

### (4-2) Synthesis of ET-4

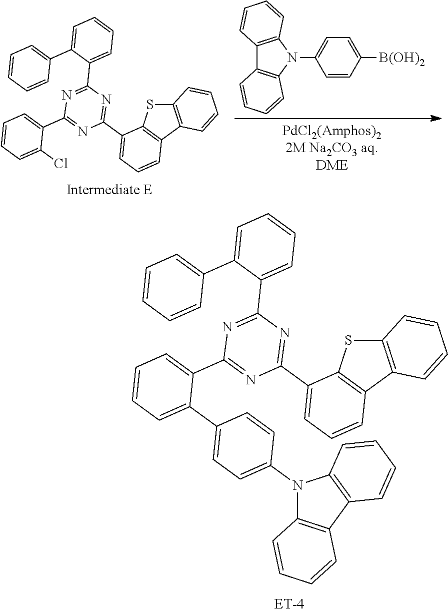

Intermediate E (5.0 g) and 4-(N-carbazolyl)phenylboronic acid (2.6 g) were used to obtain ET-4 as a white solid (5.3 g, yield: 80%) under the same conditions as described in Synthetic Example 1.

The result of mass spectral analysis was m/e=732 for a molecular weight of 732.91, thereby the product was identified as the target compound.

### (Synthesis Example 5) Synthesis of ET-5

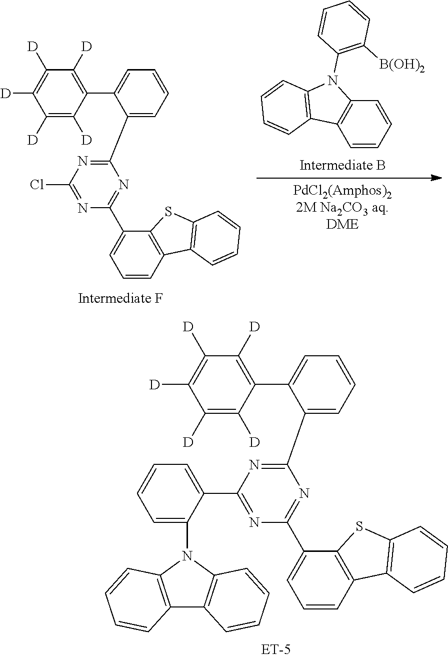 [SMILES: [2H]c1c([2H])c([2H])c(-c2ccccc2-c2nc(Cl)nc(-c3cccc4c3sc3ccccc34)n2)c([2H])c1[2H] ; OB(O)c1ccccc1-n1c2ccccc2c2ccccc21 ; [2H]c1c([2H])c([2H])c(-c2ccccc2-c2nc(-c3ccccc3-n3c4ccccc4c4ccccc43)nc(-c3cccc4c3sc3ccccc34)n2)c([2H])c1[2H]]

Intermediate F (3.0 g) synthesized in accordance with the method described in WD 2021/033724 was used to obtain ET-5 as a white solid (3.0 g, yield: 69%) under the same conditions as described in Synthesis Example 1.

The result of mass spectral analysis was m/e=661 for a molecular weight of 661.84, thereby the product was identified as the target compound.

### (Synthesis Example 6) Synthesis of ET-6

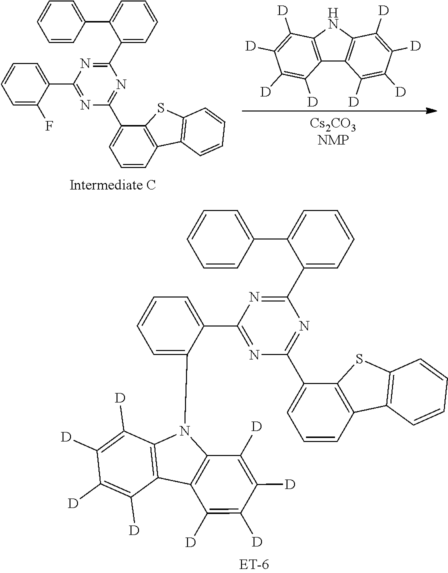 [SMILES: Fc1ccccc1-c1nc(-c2ccccc2-c2ccccc2)nc(-c2cccc3c2sc2ccccc23)n1 ; [2H]c1c([2H])c([2H])c2c([nH]c3c([2H])c([2H])c([2H])c([2H])c32)c1[2H] ; [2H]c1c([2H])c([2H])c2c(c1[2H])c1c([2H])c([2H])c([2H])c([2H])c1n2-c1ccccc1-c1nc(-c2ccccc2-c2ccccc2)nc(-c2cccc3c2sc2ccccc23)n1]

Intermediate C (4.0 g), carbazole-1,2,3,4,5,6,7,8-d8 (1.5 g), and cesium carbonate (9.0 g) were added to N-methylpyrrolidone (NMP) (50 mL), and the mixture was heated at 150° C. for 24 hours with stirring under an argon atmosphere. The solid precipitated by addition of methanol and water was dissolved in toluene and passed through silica gel. The discharged liquid was concentrated, and the resulting solid was subjected to suspension wash with ethanol, and further purified by recrystallization with toluene repeatedly to obtain ET-6 as a white solid (2.9 g, yield: 55%).

The result of mass spectral analysis was m/e=664 for a molecular weight of 664.86, thereby the product was identified as the target compound.

Although only some exemplary embodiments and/or examples of this invention have been described in detail above, those skilled in the art will readily appreciate that many modifications are possible in the exemplary embodiments and/or examples without materially departing from the novel teachings and advantages of this invention. Accordingly, all such modifications are intended to be included within the scope of this invention.

The documents described in the specification and the specification of Japanese application(s) on the basis of which the present application claims Paris convention priority are incorporated herein by reference in its entirety.

1. A compound represented by the following formula (1): 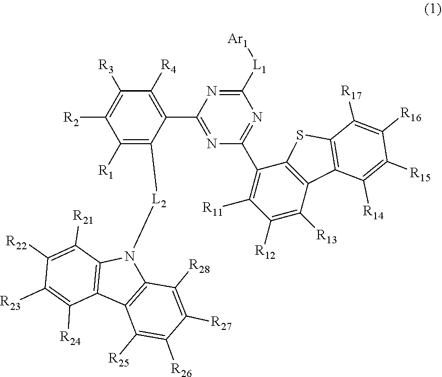
wherein in the formula (1),
Ar₁ is a substituted or unsubstituted aryl group including 6 to 50 ring carbon atoms;
L₁ and L₂ are independently a single bond or a substituted or unsubstituted arylene group including 6 to 50 ring carbon atoms;
R₁ to R₄ are independently a hydrogen atom, a substituted or unsubstituted alkyl group including 1 to 50 carbon atoms, or a substituted or unsubstituted aryl group including 6 to 50 ring carbon atoms; one or more sets of adjacent two or more of R₁ to R₄ do not form a ring by bonding with each other;
one or more sets of adjacent two or more of R₁₁ to R₁₇ form a substituted or unsubstituted, saturated or unsaturated ring by bonding with each other, or do not form a substituted or unsubstituted, saturated or unsaturated ring; R₁₁ to R₁₇ which do not form the substituted or unsubstituted, saturated or unsaturated ring are independently a hydrogen atom or a substituent R;
R₂₁ and R₂₂ form a substituted or unsubstituted, saturated or unsaturated ring by bonding with each other, or do not form a substituted or unsubstituted, saturated or unsaturated ring;
One or more sets of adjacent two or more of R₂₃ to R₂₆ form a substituted or unsubstituted, saturated or unsaturated ring by bonding with each other, or do not form a substituted or unsubstituted, saturated or unsaturated ring;
R₂₇ and R₂₈ form a substituted or unsubstituted, saturated or unsaturated ring by bonding with each other, or do not form a substituted or unsubstituted, saturated or unsaturated ring;
R₂₂ and R₂₃ do not form a ring by bonding with each other;
R₂₆ and R₂₇ do not form a ring by bonding with each other;
R₂₁ to R₂₈ which do not form the substituted or unsubstituted, saturated or unsaturated ring are independently a hydrogen atom or a substituent R;
the substituent R is selected from the group consisting of
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇)
(where R₉₀₁ to R₉₀₇ are independently
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms; and when two or more of each of R₉₀₁ to R₉₀₇ are present, the two or more of each of R₉₀₁ to R₉₀₇ may be the same as or different from each other),
a halogen atom, a cyano group, a nitro group,
a substituted or unsubstituted aryl group including 6 to 50 ring carbon atoms, and
a substituted or unsubstituted monovalent heterocyclic group including 5 to 50 ring atoms; and
when two or more substituent R's are present, the two or more substituent R's may be the same as or different from each other.

2. The compound according to claim 1, wherein Ar₁ is a group represented by the following formula (1A): 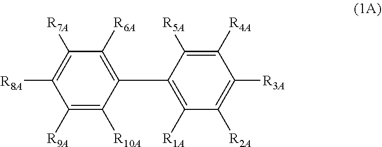
wherein in the formula (1A),
one of R_{1A} to R_{10A} represents a bond which bonds with L₁ in the formula (1);
adjacent two or more of R_{1A} to R_{10A} which do not represent the bond which bonds with L₁ form a substituted or unsubstituted, saturated or unsaturated ring by bonding with each other, or do not form a substituted or unsubstituted, saturated or unsaturated ring;
R_{1A} to R_{10A} which do not represent the bond which bonds with L₁ and do not form the substituted or unsubstituted, saturated or unsaturated ring are independently a hydrogen atom or a substituent R; and
the substituent R is as defined in the formula (1).

3. The compound according to claim 2, wherein R_{1A} represents the bond which bonds with L₁ in the formula (1A).

4. The compound according to claim 1, wherein L₁ is a single bond.

5. The compound according to claim 1, wherein the compound represented by the formula (1) is a compound represented by the following formula (1-1): 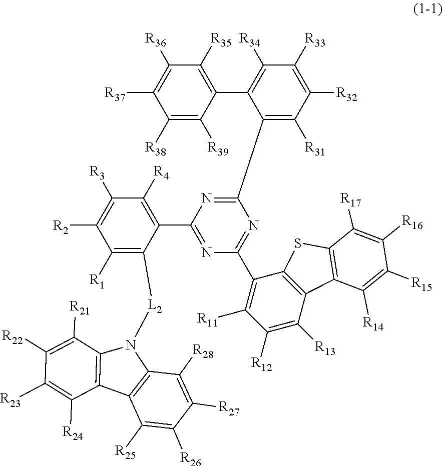
wherein in the formula (1-1),
L₂, R₁ to R₄, R₁₁ to R₁₇, and R₂₁ to R₂₈ are as defined in the formula (1);
one or more sets of adjacent two or more of R₃₁ to R₃₉ form a substituted or unsubstituted, saturated or unsaturated ring by bonding with each other, or do not form a substituted or unsubstituted, saturated or unsaturated ring;
R₃₁ to R₃₉ which do not form the substituted or unsubstituted, saturated or unsaturated ring are independently a hydrogen atom or a substituent R; and
the substituent R is as defined in the formula (1).

6. The compound according to claim 1, wherein R₁ to R₄ are hydrogen atoms.

7. The compound according to claim 1, wherein sets of adjacent two or more of R₁₁ to R₁₇ do not form a ring by bonding with each other, sets of adjacent two or more of R₃₁ to R₃₉ do not form a ring by bonding with each other, and sets of adjacent two or more of R₂₁ to R₂₈ do not form a ring by bonding with each other.

8. The compound according to claim 1, wherein R₁₁ to R₁₇ are hydrogen atoms.

9. The compound according to claim 5, wherein the compound represented by the formula (1-1) is a compound represented by the following formula (1-11): 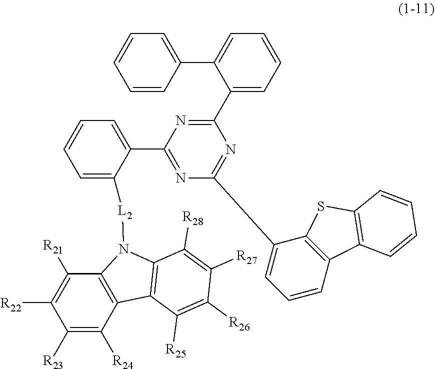
wherein in the formula (1-11), L₂ and R₂₁ to R₂₈ are as defined in the formula (1-1).

10. The compound according to claim 5, wherein the compound represented by the formula (1-1) is a compound represented by the following formula (1-12): 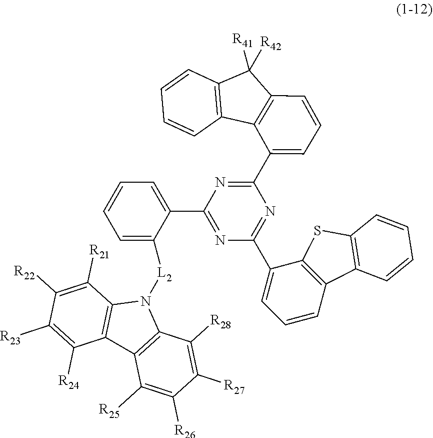
wherein in the formula (1-12), L₂ and R₂₁ to R₂₈ are as defined in the formula (1-1);
R₄₁ and R₄₂ are independently a hydrogen atom or a substituent R; and
the substituent R is as defined in the formula (1).

11. The compound according to claim 10, wherein R₄₁ and R₄₂ are independently a substituted or unsubstituted alkyl group including 1 to 50 carbon atoms, or a substituted or unsubstituted aryl group including 6 to 18 ring carbon atoms.

12. The compound according to claim 1, wherein one or more sets of adjacent two or more of R₂₁ to R₂₈ do not form a ring by bonding with each other.

13. The compound according to claim 1, wherein R₂₁ to R₂₈ are independently a hydrogen atom, a substituted or unsubstituted alkyl group including 1 to 50 carbon atoms, or a substituted or unsubstituted aryl group including 6 to 18 ring carbon atoms.

14. The compound according to claim 1, wherein R₂₁ to R₂₈ are hydrogen atoms.

15. The compound according to claim 1, wherein L₂ is a single bond.

16. The compound according to claim 1, wherein L₂ is a substituted or unsubstituted arylene group including 6 to 18 ring carbon atoms.

17. The compound according to claim 1, wherein sets of adjacent two or more of R₁₁ to R₁₇ do not form a ring by bonding with each other, and sets of adjacent two or more of R₂₁ to R₂₈ do not form a ring by bonding with each other.

18. The compound according to claim 1, wherein the compound represented by the formula (1) has at least one deuterium atom.

19. The compound according to claim 1, wherein the substituent in the case of the “substituted or unsubstituted” and the substituent R are independently a groups selected from the group consisting of
an alkyl group including 1 to 50 carbon atoms,
an aryl group including 6 to 50 ring carbon atoms, and
a heterocyclic group including 5 to 50 ring atoms.

20. The compound according to claim 1, wherein the substituent in the case of the “substituted or unsubstituted” and the substituent R are independently a groups selected from the group consisting of
an alkyl group including 1 to 18 carbon atoms,
an aryl group including 6 to 18 ring carbon atoms, and
a heterocyclic group including 5 to 18 ring atoms.

21. The compound according to claim 1, which is a material for an organic electroluminescence device.

22. An organic electroluminescence device comprising
a cathode;
an anode; and
one or two or more organic layers disposed between the cathode and the anode, wherein
at least one of the organic layers comprise the compound according to claim 1.

23. The organic electroluminescence device according to claim 22, comprising an anode, an emitting layer, an electron-transporting zone, and a cathode in this order, wherein
the electron-transporting zone comprises the compound.

24. The organic electroluminescence device according to claim 23, wherein the electron-transporting zone comprises a first layer and a second layer in this order from the emitting layer side, and
the second layer comprises the compound.

25. The organic electroluminescence device according to claim 24, wherein the second layer comprises:
the compound, and
one or more compounds selected from the group consisting of a compound containing an alkali metal and a compound containing a metal belonging to Group 13 in the Periodic Table of the Elements.

26. An electronic apparatus equipped with the organic electroluminescence device according to claim 22.
